(19) United States
(12) Patent Application Publication
(10) Pub. No.: US 2022/0133753 A1
(43) Pub. Date: May 5, 2022

(54) COMPOSITIONS AND METHODS USEFUL FOR TREATING DISEASES CHARACTERIZED BY INSUFFICIENT PANTOTHENATE KINASE ACTIVITY

(71) Applicants: Comet Therapeutics, Inc., Cambridge, MA (US); RIJKSUNIVERSITEIT GRONINGEN, Groningen (NL); ACADEMISCH ZIEKENHUIS GRONINGEN, Groningen (NL); Oregon Health & Science University, Portland, OR (US)

(72) Inventors: Gregor KOSEC, Ljubljana (SI); Ajda Podgorsek BERKE, Ljubljana (SI); Hrvoje PETKOVIC, Ljubljana (SI); Oda Cornelia Maria SIBON, Groningen (NL); Balaji SRINIVASAN, Groningen (NL); Susan J. HAYFLICK, Portland, OR (US)

(21) Appl. No.: 17/340,665

(22) Filed: Jun. 7, 2021

Related U.S. Application Data
(63) Continuation of application No. 17/083,574, filed on Oct. 29, 2020, now abandoned.
Application No. 17/083,574 is a continuation of application No. 16/309,983, filed on Dec. 14, 2018, now abandoned.
(60) Provisional application No. 62/350,878, filed on Jun. 16, 2016.

(51) Int. Cl. A61K 31/661; A61P 1/14

(52) U.S. Cl. CPC A61K 31/661; A61P 1/14

(57) ABSTRACT

Methods and pharmaceutical compositions for use in treating diseases associated with insufficient activity of the pantothenate kinase enzyme (e.g., CASTOR diseases) are disclosed. The methods and compositions involve an effective amount of an active derivative of 4′-phosphopantetheine.

### RELATED APPLICATION

This application is a continuation of U.S. application Ser. No. 17/083,574, filed Oct. 29, 2020, which is a continuation of U.S. application Ser. No. 16/309,983, filed Dec. 14, 2018, which is a U.S. National Phase application, filed under 35 U.S.C. § 371, of PCT Application No. PCT/US2017/037988, filed Jun. 16, 2017, which claims the benefit of and priority to U.S. Provisional Patent Application No. 62/350,878, filed on Jun. 16, 2016, each of which are hereby incorporated by reference in their entireties.

### INCORPORATION-BY-REFERENCE OF SEQUENCE LISTING

The contents of the file named “TM3T-003_C02US_ST25.txt”, which was created on Oct. 28, 2020, and is 1.61 KB in size are hereby incorporated by reference in their entirety.

### FIELD OF THE APPLICATION

The present application relates to compounds that can be used to treat diseases characterized by imbalances in Coenzyme A (CoA) activity and, more specifically, relates to compounds that can be used to treat Coenzyme A sequestration, toxicity or redistribution (CASTOR) diseases.

### BACKGROUND

As a carrier of acyl groups, CoA is essential for over 100 metabolic reactions, and it has been estimated that CoA is an obligatory cofactor for 4% of known enzymatic reactions. Current understanding of the de novo biosynthetic route to CoA in cells and organisms may be summarized as a specific sequential order of enzymatic activities result in the formation of CoA from Vitamin B5 (FIG. 1A). These enzymes are, in order, pantothenate kinase (PANK); phosphopantothenoyl cysteine synthetase (PPCS); phospho-N-pantothenoylcysteine decarboxylase (PPCDC); phosphopantetheine adenylyltransferase (PPAT) and dephosphoCoA kinase (DPCK). In some organisms, including Drosophila melanogaster, mice and humans, PPAT and DPCK enzyme activities are combined into a single bifunctional protein, referred to as CoA synthase (COASY). Alternatively, it has been shown in vitro that pantetheine can be phosphorylated by pantothenate kinase activity to form 4′-phosphopantetheine, which can serve as a precursor for CoA. However, direct evidence that intact pantetheine is taken up by cells and utilized for CoA biosynthesis is still lacking.

Previously, the biosynthetic route to CoA has gained attention because of its connection with specific forms of neurodegenerative diseases classified as Neurodegeneration with Brain Iron Accumulation (NBIA). These NBIAs include disorders caused by mutations in the gene encoding PANK2 (one of four human PANK genes), namely pantothenate kinase-associated neurodegeneration (PKAN). More recently, NBIA disorders caused by mutations in the gene encoding COASY were also identified, namely COASY protein-associated neurodegeneration (CoPAN). These findings suggests that impairment of the classic CoA biosynthetic route underlies progressive neurodegeneration in these patient groups. Currently, there is no treatment available to halt or reverse the neurodegeneration in these CoA-related disorders.

More recently, CoA has also gained attention due to its connection with CoA sequestration, toxicity and redistribution (CASTOR) diseases. Such diseases may be caused by accumulation of one or more acyl-CoA species to high levels. CASTOR diseases are a major challenge for clinical metabolic genetics. Currently, there are no optimal available therapies for treating CASTOR diseases.

### SUMMARY

The present disclosure provides a new approach that overcomes the drawbacks associated with previous.

The present application features, inter alia, an active derivative of 4′-phosphopantetheine for use in the treatment of a diseased subject having a Coenzyme A sequestration, toxicity or redistribution (CASTOR) disease.

In some embodiments, the diseased subject has one or more deficient, defective, and/or absent pantothenate kinases. In some embodiments, the diseased subject has one or more aberrantly expressed pantothenate kinases.

In some embodiments, the CASTOR disease is not associated with deficiency, defectiveness, and/or absence of one or more pantothenate kinases. In some embodiments, the CASTOR disease is not associated with aberrant expression of one or more pantothenate kinases. In some embodiments, the diseased subject does not have one or more deficient, defective, and/or absent pantothenate kinases. In some embodiments, the diseased subject does not have one or more aberrantly expressed pantothenate kinases. In certain embodiments, the diseased subject does not have a pantothenate kinase-associated neurodegeneration (PKAN) disease.

The CASTOR disease may be associated with inhibition of one or more pantothenate kinases by one or more acyl Coenzyme A (acyl-CoA) species.

In some embodiments, the CASTOR disease is associated with accumulation of one or more acyl Coenzyme A (acyl-CoA) species in the diseased subject to amounts greater than that of a healthy subject not having the CASTOR disease. In some embodiments, the CASTOR disease is associated with decrease of CoA and/or acetyl-CoA in the diseased subject to amounts lower than that of a healthy subject not having the CASTOR disease. In some embodiments, the CASTOR disease is associated with impaired or inhibited degradation of the one or more acyl-CoA species in the diseased subject. In certain embodiments, the one or more acyl-CoA species are not acetyl Coenzyme A (acetyl-CoA).

In some embodiments, the CASTOR disease is associated with accumulation of one or more fatty acids in the diseased subject to amounts greater than that of a healthy subject not having the CASTOR disease. In some embodiments, the CASTOR disease is associated with impaired or inhibited degradation of the one or more fatty acids in the diseased subject.

For example, the CASTOR disease is selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

For another example, the CASTOR disease may be selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, and PLA2G6-associated neurodegeneration.

For yet another example, the CASTOR disease may be selected from the group consisting of glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency /Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA: amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

For yet another example, the CASTOR disease may be selected from the group consisting of medium chain acyl-CoA dehydrogenase deficiency, short chain acyl-CoA dehydrogenase deficiency, very long chain acyl-CoA dehydrogenase deficiency, and D-bifunctional protein deficiency. For yet another example, the CASTOR disease may be medium chain acyl-CoA dehydrogenase deficiency. For yet another example, the CASTOR disease may be short chain acyl-CoA dehydrogenase deficiency. For yet another example, the CASTOR disease may be very long chain acyl-CoA dehydrogenase deficiency. For yet another example, the CASTOR disease may be D-bifunctional protein deficiency.

For yet another example, the CASTOR disease may be selected from the group consisting of Glutaric acidemia type 1, methylmalonic academia, propionyl-CoA carboxylase deficiency, propionic academia, 3-methylcrotonyl carboxylase deficiency, and isovaleryl-CoA dehydrogenase deficiency. For yet another example, the CASTOR disease may be Glutaric acidemia type 1. For yet another example, the CASTOR disease may be methylmalonic academia. For yet another example, the CASTOR disease may be propionyl-CoA carboxylase deficiency. For yet another example, the CASTOR disease may be propionic academia. For yet another example, the CASTOR disease may be 3-methylcrotonyl carboxylase deficiency. For yet another example, the CASTOR disease may be isovaleryl-CoA dehydrogenase deficiency.

The active derivative of 4′-phosphopantetheine may be a compound of Formula (I):

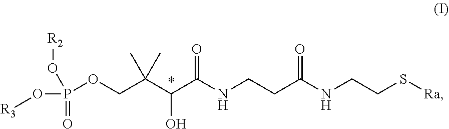

a pharmaceutically acceptable salt thereof, or a solvate thereof, wherein:

Ra is H,

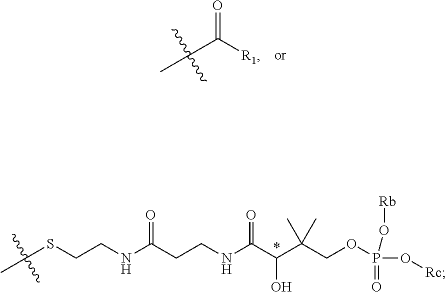

R₁is H, substituted or unsubstituted alkyl, substituted or unsubstituted alkenyl, substituted or unsubstituted cycloalkyl, substituted or unsubstituted aryl, substituted or unsubstituted arylalkyl, substituted or unsubstituted non-aromatic heterocyclyl, substituted or unsubstituted heterocyclyl, substituted or unsubstituted heterocyclylalkyl, COR₁₁, C(O)OR₁₁, C(O)NR₁₁R₁₂, C═NR₁₁, CN, OR₁₁, OC(O)R₁₁, NR₁₁R₁₂, NR₁₁C(O)R₁₂, NO₂, N═CR₁₁R₁₂, or halogen;

R₂, R₃, Rb, and Rc is each independently selected from the group consisting of H, methyl, ethyl, phenyl, acetoxymethyl (AM), pivaloyloxymethyl (POM),

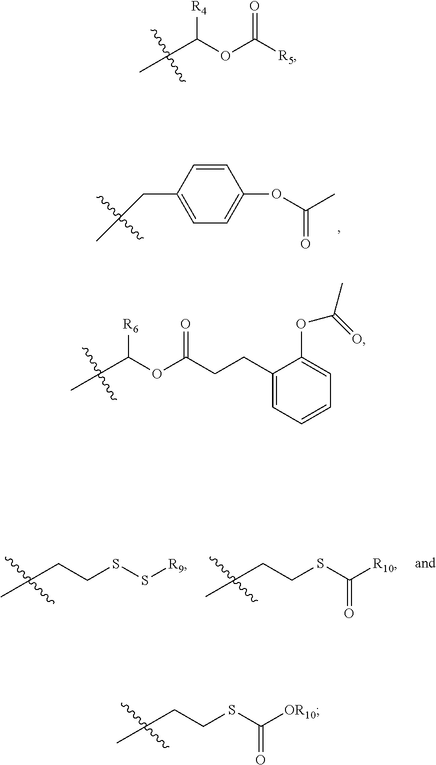

or

R₂ and R₃, or Rb and Rc, jointly form a structure selected from the group consisting of

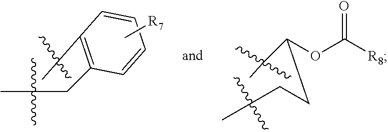

wherein

R₄ is H or alkyl;

R₅ is H or alkyl;

R₆ is H, alkyl, or CH₂(CO)OCH₃;

R₇ is H, alkyl, or halogen;

R₈ is H or alkyl;

R₉ is H or alkyl;

R₁₀ is H or-alkyl;

R₁₁ and R₁₂ each is hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, or halogen.

In some embodiments, the compound of Formula (I) is a compound of Formula (Ia):

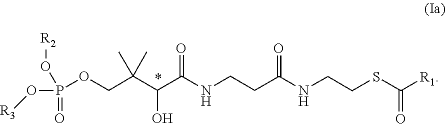

In some embodiments, R₁ is C₁-C₁₀ alkyl (e.g., R₁ is methyl, ethyl, n-propyl, i-propyl, n-butyl, s-butyl, or t-butyl). For example, R₁ is methyl.

In some embodiments, at least one of R₂ and R₃ is H. For example, one of R₂ and R₃ is H, or R₂ and R₃ are H.

For example, the active derivative of 4′-phosphopantetheine is 4′-phosphopantetheine or a pharmaceutically acceptable salt thereof. For another example, the active derivative of 4′-phosphopantetheine is S-acyl-4′-phosphopantetheine or a pharmaceutically acceptable salt thereof. For yet another example, the active derivative of 4′-phosphopantetheine is S-acetyl-4′-phosphopantetheine or a pharmaceutically acceptable salt thereof. For yet another example, the active derivative of 4′-phosphopantetheine is S-acetyl-4′-phosphopantetheine. For yet another example, the active derivative of 4′-phosphopantetheine is a salt of S-acetyl-4′-phosphopantetheine. For yet another example, the active derivative of 4′-phosphopantetheine is a calcium salt of S-acetyl-4′-phosphopantetheine.

In another aspect, the present application features a method of treating a diseased subject having a CASTOR disease as described above, comprising administering to the diseased subject an effective amount of an active derivative of 4′-phosphopantetheine as described above.

In yet another aspect, the present application features use of an active derivative of 4′-phosphopantetheine as described above in the manufacture of a medicament for the treatment of a diseased subject having a CASTOR disease as described above.

In yet another aspect, the present application features a pharmaceutical composition for use in the treatment of a diseased subject having a CASTOR disease as described above, comprising an effective amount of an active derivative of 4′-phosphopantetheine as described above.

In yet another aspect, the present application features a pharmaceutical kit for use in the treatment of a diseased subject having a CASTOR disease as described above, comprising an effective amount of an active derivative of 4′-phosphopantetheine as described above.

In yet another aspect, the present application features a method of synthesizing an active derivative of 4′-phosphopantetheine as described above. The method includes the steps of: i) chemically treating pantothenic acid with S-tritylcysteamine to form S-tritylpantetheine; ii) chemically treating S-tritylpantetheine with dibenzylchlorophosphate to form S-trityl-4′-dibenzylphosphopantetheine; and iii) chemically treating S-trityl-4′-dibenzylphosphopantetheine to form 4′-phosphopantetheine.

In yet another aspect, the present application features an active derivative of 4′-phosphopantetheine for use in the treatment of a diseased subject having a disease selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

In yet another aspect, the present application features a method of treating a diseased subject having a disease selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy. The method includes administering to the diseased subject an effective amount of an active derivative of 4′-phosphopantetheine.

In yet another aspect, the present application features use of an active derivative of 4′-phosphopantetheine in the manufacture of a medicament for the treatment of a diseased subject having a disease selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

The details of the present application are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

The contents of all references (including literature references, issued patents, published patent applications, and co-pending patent applications) cited throughout this application are hereby expressly incorporated herein in their entireties by reference.

### BRIEF DESCRIPTION OF THE DRAWINGS

FIG. 1A is a depiction of the canonical de novo CoA biosynthesis pathway. Vitamin B5 (pantothenate) is taken up and intracellularly converted to CoA by PANK, PPCS, PPCDC, PPAT and DPCK. In Drosophila and humans, PPAT and DPCK are combined into one protein, COASY. Abbreviations of the enzymes (in black circles) and intermediate products are indicated.

FIG. 1B is a bar graph of the Drosophila S2 cell count of control (100%) and dPANK/fbl RNAi treated cells. The insert is an image of a western blot analysis of dPANK/Fbl protein levels in control and dPANK/fbl RNAi treated cells, tubulin as loading control. Error bars indicate±SD (n=3). Unpaired t-test was used (*p≤0.05, **p≤0.01, ***p≤0.001).

FIG. 1C is a plot of cell counts of control (100%) and dPANK/fbl RNAi treated cells in the presence of increasing concentrations CoA. Error bars represent±SD (n=3).

FIG. 1D is a set of 15 images depicting protein acetylation levels visualized using immunofluorescence, in control and dPANK/fbl RNAi treated cells with and without CoA. An antibody against acetylated Lysine (green), Rhodamin-Pahlloidin (red; marking F-actin), and DAPI (blue, DNA) were used. Scale bars represent 20 um.

FIG. 1E is a plot of cell counts of control (100%) and HoPan treated cells in the presence of increasing concentrations of CoA. Error bars represent±SD (n=3).

FIG. 1F is a set of 12 images depicting protein acetylation levels visualized in control and HoPan treated cells with and without CoA. An antibody against acetylated Lysine (green), Rhodamin-Pahlloidin (red; marking F-actin), and DAPI (blue, DNA) were used. Scale bars represent 20 um.

FIG. 1G is a bar graph of the cell count of control (100%) and HoPan treated mammalian HEK293 cells with and without CoA. Error bars indicate±SD (n=3). Unpaired t-test was used (*p≤0.05, **p≤0.01).

FIG. 1H is an image of a Western blot and a bar graph showing the quantification of histone acetylation levels in control and HoPan treated mammalian HEK293 cells in the presence and absence of CoA. GAPDH represents the loading control. Error bars represent±SD (n=3). Unpaired t-test was used (*p≤0.05).

FIG. 2A is a plot of bends per 30 seconds used to quantify motility in C. elegans pnk-1 mutant and wild type animals with and without CoA treatment. Error bars represent±SD (n=45). Unpaired t-test was used (***p≤0.001).

FIG. 2B is a plot of a lifespan analysis of C. elegans pnk-1 mutants and wild type animals (n≥100) with and without CoA treatment. Survival curves were found to be significant with p value<0.001, analyzed with Log-rank (Mantel-Cox) test, between untreated and CoA (400 uM) treated pnk-1 mutants.

FIG. 2C is a set of representative serial images demonstrating movements of C. elegans wild types and pnk-1 mutants with and without CoA treatment. Still images are given in c1, c3 and c5; and images are superimposed in c2, c4 and c6, respectively. Scale bars represent 200 μm.

FIG. 2D is a plot of the eclosion rate of adult flies as determined in control flies (set as 100%) and in flies treated with increasing concentrations of HoPan present in the food during development. Error bars represent±SD (n=3). Unpaired t-test was used (*p≤0.05, **p≤0.01).

FIG. 2E is a plot of the eclosion rate of adult flies as determined in control flies (set as 100%) and in flies treated with 2.5 mM HoPan present in the food during development, in the presence of the indicated concentrations of CoA. Error bars represent±SD (n=3).

FIG. 2F is a bar graph of intracellular CoA levels measured with HPLC analysis in Drosophila S2 control cells (100%) and cells treated with HoPan alone or with HoPan and CoA. Unpaired t-test was used (*p≤0.05) between groups. Error bars represent±SD (n=3).

FIG. 2G is a bar graph of intracellular CoA levels measured with HPLC analysis in mammalian HEK293 control cells (100%) and cells treated with HoPan alone or with HoPan and CoA. Error bars represent±SD (n=3). Unpaired t-test was used (**p≤0.01).

FIG. 3A is a bar graph of CoA levels determined by HPLC analysis in PBS (t=0 in PBS is 100%), medium, medium containing serum and in fetal calf serum after 3 hours incubation. Unpaired t-test was used (*p≤0.05). Error bars represent±SD (n=3).

FIG. 3B is a plot showing the stability profile of CoA determined by HPLC analysis in PBS (t=0 in PBS is 100%) and in fetal calf serum over the course of 6 hours. Error bars represent±SD (n=3).

FIG. 3C is a set of three HPLC chromatograms of CoA incubated for 3 hours in (c1) PBS and in (c2) fetal calf serum. (c3) Retention time of standard PPanSH is identical to the observed conversion product of CoA in serum.

FIG. 3D is a plot showing concentrations of CoA and PpanSH in mouse serum over 6 hours. Concentrations were determined by HPLC analysis. Error bars represent±SD (n=3).

FIG. 3E is a plot showing concentrations of CoA and PpanSH in human serum over 6 hours. Concentrations were determined by HPLC analysis. Error bars represent±SD (n=3).

FIG. 3F is a bar graph of Relative PpanSH levels in Drosophila L1 and L2 stage larvae determined by HPLC analysis under control conditions (100%) and after feeding CoA. Error bars represent±SD (n=3). Unpaired t-test was used (**p≤0.01, ***p≤0.001).

FIG. 3G is a bar graph showing the concentration of CoA and PpanSH at 30 minutes in mice determined by HPLC analysis after in vivo injecting the indicated amounts of CoA intravenously. Error bars represent±SD.

FIG. 4A is a bar graph showing the results where fetal calf serum, mouse serum and human serum were heat-inactivated, and CoA levels were measured after 3 hours using HPLC analysis.

FIG. 4B is a bar graph showing the results where fetal calf serum, mouse serum and human serum were treated with EDTA, and CoA levels were measured after 3 hours using HPLC analysis.

FIG. 4C is a bar graph showing the results where fetal calf serum, mouse serum and human serum were treated with ATP and ADP as indicated, and CoA levels were measured after 3 hours using HPLC analysis.

FIG. 4D is a bar graph showing the results where fetal calf serum, mouse serum and human serum were pre-treated with sodium fluoride (NaF), levamisole, suramin, 4,4′-diisothiocyanatostilbene-2,2′ disulphonic acid (DIDS) and CoA levels were measured. (PpanSH=4′-phosphopantetheine; in all panels CoA was added to the indicated sera with a final starting concentration of 10 μM measured by HPLC analysis and percentages relative to CoA incubation for 3 hours in PBS (=100%) are indicated on the y-axis).

For FIGS. 4A-4D, unpaired t-test was used (***p≤0.001). Error bars represent±SD (n=3).

FIG. 5A is a bar graph showing the measurement of intracellular PpanSH levels by HPLC analysis in control Drosophila S2 cells (100%) and cells treated with HoPan with and without addition of CoA or PpanSH.

FIG. 5B is a plot of the Drosophila S2 cell count determined in control cells (100%) and HoPan treated cells at the indicated PpanSH concentrations.

FIG. 5C is a bar graph showing the cell count determined in control (100%) and dPANK/fbl RNAi treated Drosophila S2 cells with and without addition of PpanSH to the medium as indicated.

FIG. 5D is a bar graph showing the cell count of mammalian HEK293 control cells (100%), cells treated with HoPan with and without CoA or PpanSH added to the medium.

FIG. 5E is a bar graph showing the relative CoA levels of control (100%) and HoPan treated HEK293 cells with and without CoA or PpanSH added to the medium as determined by HPLC.

FIG. 5F is an image of a Western blot analysis and a bar graph of the quantification to determine histone acetylation levels of control HEK293 cells, cells treated with HoPan with and without CoA or PpanSH.

FIG. 5G is a bar graph of the results from S2 cells, with and without HoPan incubated with PpanSH(D4). Levels of both unlabeled CoA and labelled CoA(D4) were measured. Cumulative CoA and CoA(D4) levels were considered for statistical analysis.

FIG. 5H is a plot of PpanSH labelled with 4 deuterium atoms (PpanSH(D4)) added to S2 cells at 4° C. and 25° C. and incubated for the indicated times. Mass spectrometry was used to measure levels of labelled compound in harvested cell extracts.

FIG. 5I is a bar graph of the results from S2 cells incubated with PpanSH(D4) incubated with the indicated concentrations of PpanSH.

FIG. 6A is a plot of a lifespan analysis of control and hypomorphic (dPANK/fbl¹) homozygous mutant flies (n≥85) with and without CoA treatment. Survival curves were found to be significant with p value<0.001, analyzed with Log-rank (Mantel-Cox) test, between untreated and CoA (9 mM) treated dPANK/fbl¹ mutants.

FIG. 6B is a bar graph of the number of progeny in the form of pupae produced by homozygous null (dPANK/fblnull) mutants with and without treatment with the indicated concentrations of CoA and Vitamin B5.

FIG. 6C is a bar graph of the number of progeny of homozygous dPPCDC mutants in the form of developed pupae with and without addition of CoA or Vitamin B5.

FIG. 6D is a plot of a lifespan analysis of female flies of the dPPCDC RNAi line with and without treatment of CoA or Vitamin B5. The p value<0.001, as analyzed with Log-rank (Mantel-Cox) test.

FIG. 6E is a set of images showing of ovary size of 4-day old control and females of dPPCDC RNAi Drosophila line untreated, or treated with CoA or Vitamin B5, imaged with light microscopy. Scale bars represent 200 μm.

FIG. 6F is a bar graph showing the number of eclosed adult progeny of dPPCDC RNAi females when crossed with control males with and without addition of Vitamin B5 or CoA.

FIG. 6G is a bar graph showing the number of L₁ and L2 larvae of homozygous dCOASY mutants and control larvae with and without the treatment of CoA or Vitamin B5.

FIG. 6H is a bar graph and image of a Western blot showing the results where RNAi was used to down-regulate COASY in HEK293 cells treated or not treated with CoA as indicated. The Western blot shows successful down-regulation of human COASY by RNAi and decreased histone acetylation (and quantification). GAPDH represents the loading control.

FIG. 6I is a depiction of a non-canonical CoA supply route with extracellular CoA as starting point. ENPP represents ecto-nucleotide pyrophosphatases.

For FIGS. 6B, 6F and 6G, error bars represent±SD (n=3). Unpaired t-test was used (*p≤0.05, **p≤0.01, ***p≤0.001). Solid thick bars without error bars represent that no pupae or eclosed flies were observed.

FIG. 7A is a plot showing the quantification of motility in C. elegans pantothenate kinase (pnk-1) mutants with and without addition of the indicated CoA concentrations to the food. Error bars represent±SD (n≥15). Unpaired t-test was used to assess statistical significance (*p≤0.05, **p≤0.01, ***p≤0.001).

FIG. 7B is a plot showing the lifespan analysis of C. elegans pnk-1 mutants (n≥100) with and without CoA treatment (100 and 400 uM). Survival curves were found to be significant with p value<0.001, analyzed with Log-rank (Mantel-Cox) test, between control and CoA treated pnk-1 mutants.

FIG. 8 is a depiction of the synthesis of 4′-phosphopantetheine from pantothenate through coupling, phosphorylation and deprotection steps.

FIG. 9 is a set of five HPLC chromatograms showing CoA stability in PBS and fetal calf serum compared with standard 4′-phosphopantetheine (PpanSH), Panetheine and Dephospho-CoA. CoA is migrating at 17.65 min; PpanSH at 18.27 min; Pantetheine at 21.61 min and Dephospho-CoA at 18.85 min. CoA is stable in PBS and converted in serum in a thiol-containing compound exactly migrating as PpanSH standard at 18.27 min. Chemical structures of CoA, PpanSH, Pantetheine and Dephospho-CoA are presented.

FIG. 10A is an HPLC chromatogram profile in untreated fresh mouse serum (solid line), that shows a peak which comigrates exactly with PpanSH as visible when the sample was spiked with standard PpanSH (dotted line). These results indicate the presence of endogenous PpanSH.

FIG. 10B is a plot of mass spectrometry results of a PpanSH standard.

FIG. 10C is a plot of mass spectrometry results showing endogenous PpanSH in mouse plasma.

FIG. 10D is a plot of mass spectrometry results used to confirm the presence of elevated levels of PpanSH in plasma, 6 hrs after CoA injection (0.5 mg) in mice.

FIG. 11A is a bar graph showing the amount of 4′-phosphopantetheine in fetal calf serum that was heat-inactivated or pre-treated with EDTA, or ATP or ADP, or with the inhibitors Sodium fluoride (NaF) or Suramin as indicated as measured in FIGS. 4A-4C above.

FIG. 11B is a bar graph showing the amount of 4′-phosphopantetheine in mouse serum that was heat-inactivated or pre-treated with EDTA, or ATP or ADP, or with the inhibitors Sodium fluoride (NaF) or Suramin as indicated as measured in FIGS. 4A-4C above.

FIG. 11C is a bar graph showing the amount of 4′-phosphopantetheine in human serum that was heat-inactivated or pre-treated with EDTA, or ATP or ADP, or with the inhibitors Sodium fluoride (NaF) or Suramin as indicated as measured in FIGS. 4A-4C above.

For FIGS. 11A-11C, error bars represent±SD (n=3), and solid black bars without error bars represent that no PpanSH was detected.

FIG. 12A is a set of 15 images depicting the use of immunofluorescence to visualize protein acetylation levels in control and dPANK/fbl RNAi treated S2 cells with and without PpanSH. An antibody against acetylated Lysine (green), Rhodamin-Pahlloidin (red; marking F-actin), and DAPI (blue, DNA) were used. Addition of PpanSH rescues acetylation defects of dPANK/fbl RNAi treated S2 cells.

FIG. 12B is a set of 15 images depicting the use of immunofluorescence to visualize protein acetylation levels in control and HoPan treated S2 cells with and without PpanSH. An antibody against acetylated Lysine (green), Rhodamin-Pahlloidin (red; marking F-actin), and DAPI (blue, DNA) were used. Addition of PpanSH rescues acetylation defects of dPANK/fbl RNAi treated S2 cells.

FIG. 13A is a plot showing the results of mass spectrometry was used to detect the presence and levels of 4′-phosphopantetheine labelled with stable isotope (deuterium) (PpanSH(D4)).

FIG. 13B is a plot showing the results of mass spectrometry was used to detect the presence and levels of of 4′phosphopantetheine labelled with stable isotope (deuterium) (PpanSH(D4)).

FIG. 13C is a plot showing the results of mass spectrometry was used to detect the presence and levels of 4′phosphopantetheine labelled with stable isotope (deuterium) (PpanSH(D4)).

FIG. 13D is a plot showing the results of mass spectrometry was used to detect the presence and levels of 4′phosphopantetheine labelled with stable isotope (deuterium) (PpanSH(D4)).

For FIGS. 13A-13D, S2 cells were treated with HoPan and PpanSH(D4) was added to the medium. The CoA(D4) level was measured. Together, FIGS. 13A-13D show that under control conditions PpanSH(D4) and CoA(D4) could be detected, indicating that PpanSH is taken up by cells and converted into CoA. Levels of CoA(D4) are increased under conditions of HoPan treatment compared to no HoPan treatment, underscoring the presence of a bypass route via PpanSH. Chemical structures of PpanSH(D4) and CoA(D4) are given.

FIG. 13E is a depiction of a Parallel Artificial Membrane Permeability Assay (PAMPA). Experiments were performed according to the manufacturer's instructions. In this assay, a two-well system is separated by an artificial lipid-oil-lipid membrane (shown in grey). To the lower (donor) compartment, a compound dissolved in buffer is added, the upper (acceptor) compartment contains only buffer. After 5 hours of incubation, concentration of compound is measured in both wells to assess its propensity to diffuse over the artificial membrane. The permeability was calculated according to the manufacturer's instruction (formulas are depicted to the right). Compounds that are below the assay threshold are predicted to be unable to pass membranes passively, whereas compounds above the threshold are able to pass membranes passively. Ceq=Equilibrium Concentration, CD=Concentration in donor well, VD=Volume of donor well (0.3 ml), CA=Concentration in acceptor well, VA=Volume of acceptor well (0.2 ml), P=Permeability, S=Membrane area (0.3 cm2), t=Incubation time (18000 s).

FIG. 13F is a bar graph showing that PpanSH, like the positive control caffeine, is classified as a well-permeating compound, whereas CoA, like negative control amiloride, is a poorly permeating molecule. Error bars represent±S.E.M of data using n≥4.

FIG. 14 is a depiction of the CoA biosynthesis route in which the enzymatic conversion steps 1, 2 and 3, upstream of PpanSH and the combined enzymatic step 4-5 downstream of PpanSH are indicated. For each conversion step the mutant lines and/or RNAi lines are indicated. Upper image represents time scale and images of normal Drosophila developmental and adult stages. Fly line and mutant-specific developmental arrest is indicated under control conditions (dotted line) and after CoA supplementation to the food (solid line).

FIG. 15A is a bar graph of mRNA expression levels of dPPCDC normalized with house-keeping gene (rp49) expression levels in 1-day old adult dPPCDC RNAi Drosophila female flies and in age-matched control flies.

FIG. 15B is a bar graph of mRNA expression levels of dPPCDC normalized with house-keeping gene (rp49) expression levels in L2 control larvae and in L2 dPPCDC mutant.

FIG. 15C is a bar graph of mRNA expression levels of dCOASY normalized with house-keeping gene (rp49) expression levels in L1 control larvae and in L₁ dCOASY mutant larvae.

For FIGS. 15A-15C, error bars represent±SD (n≥3). Unpaired t-test was used to assess statistical significance (*p≤0.05, **p≤0.01, ***p≤0.001).

FIG. 15D is a plot of a lifespan analysis of hypomorphic (dPANK/fbl1) homozygous mutants (n≥85) with and without the indicated concentrations of CoA (6, 9 and 12 mM) added to the food. Survival curves were found to be significant with p value<0.001, analyzed with Log-rank (Mantel-Cox) test, between control and all CoA treated dPANK/fbl1 mutants.

FIG. 15E is a plot of a lifespan analysis of adult female dPPCDC RNAi flies (n≥100) with and without various concentrations of CoA (9, 18 and 21 mM) added to the food. Survival curves were found to be significant with p value<0.01 for CoA 9 mM treatment and p value<0.001 for CoA (18 and 21 mM) treatment compared to control untreated dPPCDC RNAi mutants, analyzed with Log-rank (Mantel-Cox) test.

FIG. 16A is a set of images depicting a Western blot analysis of dPANK/Fbl protein expression levels of control animals, homozygous hypomorphic (dPANK/fbl1) mutants and homozygous null (dPANK/fblnull) mutants. Tubulin is included as a loading control.

FIG. 16B is a bar graph showing CoA and PpanSH levels measured by HPLC analysis in 1-day old hypomorphic homozygous (dPANK/fbl1) mutant and control adult flies. CoA and PpanSH levels in mutant larvae are presented as percentages of CoA levels in control larvae.

FIG. 16C is a bar graph showing CoA and PpanSH levels measured by HPLC in early L2 null homozygous (dP ANK/fblnull) mutant and control larvae. CoA levels in mutant larvae are presented as percentages of CoA levels in control larvae.

FIG. 16D is a bar graph of the relative CoA and PpanSH levels measured by HPLC in 1-day old females of the dPPCDC RNAi fly line compared to control flies.

FIG. 16E is a bar graph of CoA and PpanSH levels measured by HPLC of the L2 larval stage of control and homozygous dPPCDC mutant larvae.

FIG. 16F is a bar graph of Relative CoA and PpanSH levels measured by HPLC of 1-day old homozygous dCOASY mutant larvae, compared to control.

FIG. 16G is a bar graph of the relative levels of CoA and PpanSH were measured in control HEK293and COASY down-regulated cells treated with medium with and without addition of CoA.

FIG. 17A is a set of three images depicting ovaries of 4-day old control and dPPCDC RNAi expressing flies, stained with Rhodamin-Phalloidin (red, marking F-actin) and the nuclear marker DAPI (green) and imaged with confocal microscopy. (a1) In wild-type ovarioles strings of developing egg-chambers, from the germarium up to stage 9 were visible. Mature eggs were also found (marked by asterisks), identifiable by the presence of yolk. (a2) In ovaries of the dPPCDC RNAi expressing flies, egg-chambers developed normally until stage 7. From stage 8 on, fragmented and condensed DNA was visible indicating apoptosis (marked by blue arrowheads). No egg-chambers older than stage 8/9 or mature eggs were found in these ovaries. (a3) CoA treatment of the dPPCDC RNAi expressing flies improved egg-production significantly and eggs developed to maturity (marked by asterisks). Scale bars represent 100 μM.

FIG. 17B is a set of images showing increased fertility of dPPCDC RNAi expressing females. Untreated, Vitamin B5 treated and CoA treated dPPCDC RNAi expressing females were mated with control males and put onto apple juice plates to allow egg laying for 4 days. For untreated and Vitamin B5 treated females, no or only very few eggs were observed on the plates (compare to FIG. 6E). CoA treated females produced a significant number of eggs that developed into pupae which eclosed resulting in viable offspring. Scale bars represent 1 cm.

FIG. 18A is a bar graph showing results where pantethine was incubated for 15 min at 37° C. in fetal calf serum, mice serum and human serum and levels of total pantetheine and cysteamine were measured using HPLC.

FIG. 18B is a bar graph showing the concentration of PpanSH in various food sources (yeast, E. coli and mouse liver) levels of CoA and PpanSH were measured and found to be present. Error bars represent±SD (n=3).

FIG. 19 is a plot showing the oxidative respiration reserve capacity of primary fibroblasts from apparently healthy controls, and medium-chain acyl-CoA dehydrogenase (MCAD) deficiency patients, in response to S-acetyl-4′-phosphopantetheine treatment. Each treatment was performed in duplicate. Error bars indicate the standard deviation, and linear trendlines are displayed.

FIG. 20A is a plot outlining a mitochondrial stress test protocol with indication of chemical additions.

FIG. 20B is a plot showing basal oxygen consumption rate (OCR) levels obtained in primary human fibroblasts from apparently healthy controls, and patients diagnosed with MCAD deficiency or propionic acidemia (PA) deficiency, in response to S-acetyl-4′-phosphopantetheine treatment. Each treatment was performed in duplicate. Error bars indicate the standard deviation, and logarithmic trendlines are displayed.

FIG. 20C is a plot showing representative OCR levels obtained in primary human fibroblasts from apparently healthy controls in response to S-acetyl-4′-phosphopantetheine treatment.

FIG. 20D is a plot showing representative OCR levels obtained in primary human fibroblasts from patients diagnosed with PA deficiency in response to S-acetyl-4′-phosphopantetheine treatment.

FIG. 21 is a graph showing the area under the curve (AUC) generated from the cumulative survival percentage of drosophila for an RNAi mutant model of very-long-chain acyl-CoA dehydrogenase (VLCAD) deficiency and control drosophila. Negative values represent a reduced capacity of the mutant flies to survive in starvation, which is partially recovered towards control levels after treatment with 5 mM S-acetyl-4′-phosphopantetheine.

FIG. 22A is a plot showing the cumulative percentage eclosion over time for an RNAi knock-down (KD) drosophila model of 3-methylcrotonyl-CoA carboxylase (3-MCC) deficiency compared with the non-driven Cy control progeny from the same cross. This shows the clear developmental delay phenotype, with a 72 h shift in t_{1/2} of eclosion.

FIG. 22B is a graph showing the area under the curve (AUC) generated from the cumulative percentage eclosion of drosophila for an RNAi knock-down model of 3-MCC deficiency compared with control drosophila upon treatment with S-acetyl-4′-phosphopantetheine at 80 μM, 400 μM, 2 mM, and control vehicle. Negative values represent a developmental delay of the mutant flies, which is partially recovered after treatment with 2 mM S-acetyl-4′-phosphopantetheine.

### DETAILED DESCRIPTION

The metabolic cofactor Coenzyme A (CoA) has gained renewed attention because of its role in neurodegeneration, protein acetylation, autophagy and signal transduction. The longstanding dogma is that eukaryotic cells obtain this essential cofactor exclusively via the uptake of extracellular precursors, especially vitamin B5, which is then intracellularly converted through five conserved enzymatic reactions into CoA.

The present application is partially based on our discovery that ectonucleotide-pyrophosphatases hydrolyze CoA into 4′-phosphopantetheine. In contrast to pantetheine, 4′-phosphopantetheine is stable in serum, is taken up by cells via passive diffusion, and is intracellularly re-converted into CoA. Via this route, exogenous CoA rescues CoA-deprived phenotypes at the cellular, developmental, organismal and behavioral level. It is shown herein that CoA rescue is independent of the first three classic CoA biosynthetic steps (PANK, PPCS and PPCDC) and that it depends on the last bifunctional enzyme, COASY.

Our discovery thus suggests an alternate mechanism for cells and organisms to influence intracellular CoA levels derived from an extracellular CoA source with 4′-phosphopantetheine as the key intermediate. This route requires only two of the classic enzymatic steps of the de novo CoA biosynthetic route.

### Active Derivatives of 4′-Phosphopantetheine

An active derivative of a compound is a compound or portion of a compound that is derived from or is theoretically derivable from a parent compound. For example, a derivative can contain one or more substitutions of one or more atoms that differ from the original or ‘parent’ compound but still (a) share a common structural scaffold and (b) have the same, similar, or an improved function in the same reaction. Examples of derivatives of 4′-phosphopantetheine are described in Branko et al, EP2868662, published 6 May 2015. Particular reference is made to the compounds as disclosed at page 3, line 13 to page 7, line 10 of EP2868662. One can determine whether or not a derivative of 4′-phosphopantetheine is active using, for example, the methods described in the Examples below.

In a first aspect, the active derivatives of 4′-phosphopantetheine relate to a compound of Formula (I):

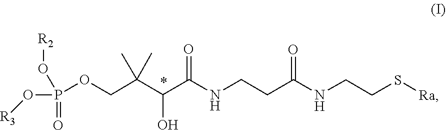

a pharmaceutically acceptable salt, or a solvate thereof,

wherein:

Rₐ is H,

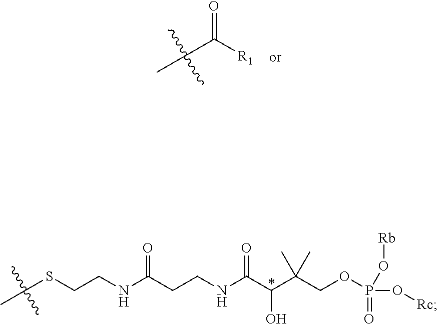

preferably

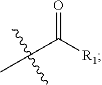

and wherein:

R₁ is H, unsubstituted or substituted alkyl, unsubstituted or substituted alkenyl, substituted or unsubstituted cycloalkyl, substituted or unsubstituted aryl, substituted or unsubstituted arylalkyl, substituted or unsubstituted non-aromatic heterocyclyl, substituted or unsubstituted aromatic heterocyclyl, substituted or unsubstituted heterocyclylalkyl, COR₁₁, C(O)OR₁₁, C(O)NR₁₁R₁₂, C═NR₁₁, CN, OR₁₁, OC(O)R₁₁, NR₁₁R₁₂, NR₁₁C(O)R₁₂, NO₂, N═CR₁₁R₁₂, or halogen; preferably C₁-C₁₀ alkyl, more preferably methyl, ethyl, propyl, or butyl (e.g., t-butyl), most preferred methyl;

R₂, R₃, R_{b} and R_{c} are independently selected from the group consisting of: H, methyl,

ethyl, phenyl, acetoxymethyl (AM), pivaloyloxymethyl (POM),

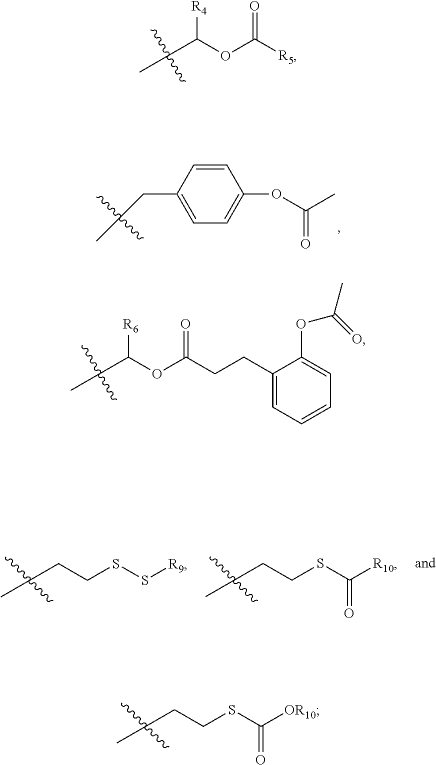

or

R₂ and R₃ or R_{b} and R_{c} jointly form a structure selected from the group consisting of:

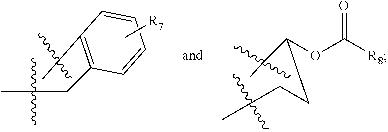

wherein
R₄ is H or alkyl, preferably -methyl;
R₅ is H or alkyl, preferably -methyl or t-butyl;
R₆ is H, alkyl, or CH₂(CO)OCH₃;
R₇ is H, alkyl, or halogen;
R₈ is H or alkyl, preferably t-butyl;
R₉ is H or alkyl, preferably -methyl or t-butyl;
R₁₀ is H or alkyl, preferably -methyl or t-butyl;
R₁₁ and R₁₂ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, or halogen.

A straight line overlayed by a wavy line denotes the covalent bond of the respective residue to the Formula (I).

The alkyl groups as described above each independently may be selected from the group consisting of methyl, ethyl, propyl (e.g., n-propyl and i-propyl), and butyl (e.g., n-butyl, s-butyl, and t-butyl).

The carbon atoms marked with “*” each independently may have D or L stereoisomeric configuration. In some embodiments, all of the carbon atoms marked with “*” have D stereoisomeric configuration.

In some embodiments, the compound of Formula (I) is a compound of Formula (Ia):

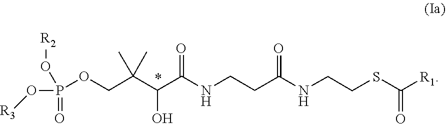

In some embodiments, R₁ is C₁-C₁₀ alkyl, e.g., methyl, ethyl, propyl (e.g., n-propyl and i-propyl), or butyl (e.g., n-butyl, s-butyl, and t-butyl). For example, R₁ is methyl.

In some embodiments, at least one of R₂ and R₃ is H.

In some embodiments, one of R₂ and R₃ is H.

In some embodiments, R₂ and R₃ are H.

In some embodiments, R₂ and R₃ are H, and R₁ is methyl.

In some embodiments, R₂, R₃, R_{b}, and R_{c} are identical residues. For example, R₂, R₃, R_{b}, and R_{c} are H, bis-POM, or bis-AM.

In some embodiments, R₂, R₃, R_{b}, and R_{c} are ethyl, or R₂, R₃, R_{b}, and R_{c} are phenyl.

In some embodiments, R₂ and R_{b} are ethyl and R₃ and R_{c} are phenyl, or R₃ and R_{c} are ethyl and R₂ and R_{b} are phenyl.

In some embodiments, R₂, R₃, R_{b} and R_{c} are all

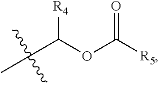

where R₄ is H or methyl, and R₅ is alkyl (e.g., methyl or t-butyl). In preferred embodiments, R₄ is H and R₅ is methyl. Hence, R₂, R₃, R_{b} and R_{c} may all be acetoxymethyl (AM). In other preferred embodiments, R₄ is H and R₅ is t-butyl. Hence, R₂, R₃, R_{b} and R_{c} may all be pivaloyloxymethyl (POM).

In some embodiments, R₂ and R₃ are

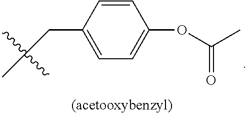 [SMILES: *Cc1ccc(OC(C)=O)cc1]

In some embodiments, R₂, R₃, R_{b}, and R_{c} are

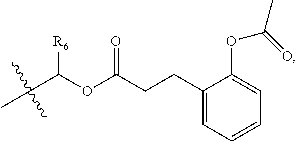

wherein R₆ is H, alkyl or CH₂(CO)OCH₃.

In some embodiments, R₂ and R₃, or R_{b} and R_{c}, jointly form

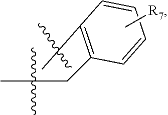

wherein R₇ is alkyl or halogen.

In some embodiments, R₂ and R₃, or R_{b} and R_{c}, jointly form

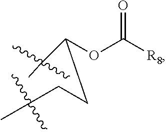

wherein R₈ is t-butyl.

In some embodiments, R₂ and R₃ are S-[(2-hydroxyethyl)sulfidyl]-2-thioethyl (DTE) or

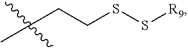

wherein R₉ is alkyl (e.g., methyl, ethyl, propyl, or butyl (e.g., t-butyl)).

In some embodiments, R₂, R₃, R_{b}, and R_{c} are S-acyl-2-thioethyl (SATE) or

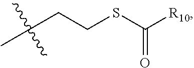

wherein R₁₀ is alkyl (e.g., methyl, ethyl, propyl, or butyl (e.g., t-butyl)).

In some preferred embodiments, the active derivative of 4′-phosphopantetheine is 4′-phosphopantetheine, a pharmaceutically acceptable salt, or a solvate thereof.

In some preferred embodiments, the active derivative of 4′-phosphopantetheine is S-acyl-4′-phosphopantetheine, a pharmaceutically acceptable salt, or a solvate thereof.

In some preferred embodiments, the active derivative of 4′-phosphopantetheine is S-propionyl-4′-phosphopantetheine, a pharmaceutically acceptable salt, or a solvate thereof.

In some preferred embodiments, the active derivative of 4′-phosphopantetheine is S-acetyl-4′-phosphopantetheine, a pharmaceutically acceptable salt, or a solvate thereof.

In some preferred embodiments, the active derivative of 4′-phosphopantetheine is a salt of S-acetyl-4′-phosphopantetheine.

In some preferred embodiments, the active derivative of 4′-phosphopantetheine is a calcium salt of S-acetyl-4′-phosphopantetheine.

In another aspect, active derivatives of 4′-phosphopantetheine include 4′-phosphopantetheine.

### Derivatives of 4′-Phosphopantothenate

In another aspect, active derivatives of 4′-phosphopantetheine also include 4′-phosphopantothenate and its derivatives. Examples of derivatives of 4′-phosphopantothenate are described in Vaino et al., WO2013163567A1, pages 3-13, published 31 Oct. 2013 and Vaino et al., WO2015061792A1, pages 13-50, published 30 Apr. 2015, which are incorporated by reference herein.

Non-limiting examples of derivatives of 4′-phosphopantothenate relate to a compound of Formula (II):

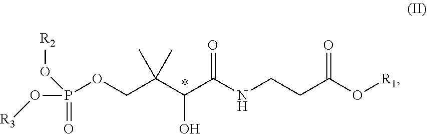

a pharmaceutically acceptable salt thereof, or a solvate thereof,

wherein:

R₁ is —H, unsubstituted or substituted alkyl, unsubstituted or substituted alkenyl, substituted or unsubstituted cycloalkyl, substituted or unsubstituted aryl, substituted or unsubstituted arylalkyl, substituted or unsubstituted non-aromatic heterocyclyl, substituted or unsubstituted aromatic heterocyclyl, substituted or unsubstituted heterocyclylalkyl, COR₁₁, C(O)OR₁₁, C(O)NR₁₁R₁₂, C═NR₁₁, CN, OR₁₁, OC(O)R₁₁, NR₁₁R₁₂, NR₁₁C(O)R₁₂, NO₂, N═CR₁₁R₁₂, or halogen; preferably C₁-C₁₀ alkyl, more preferably methyl, ethyl, propyl, or butyl, such as t-butyl, most preferred methyl;

R₂ and R₃ are independently selected from the group consisting of: H, methyl, ethyl, phenyl, acetoxymethyl (AM), pivaloyloxymethyl (POM),

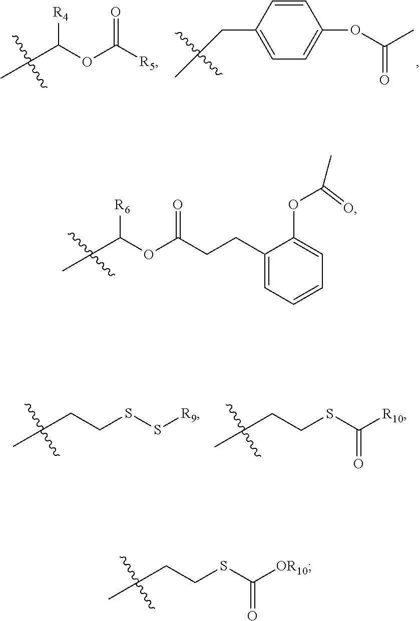

or

R₂ and R₃ jointly form a structure selected from the group consisting of:

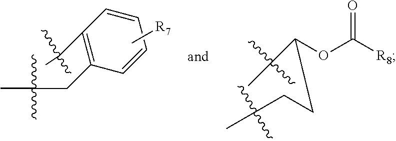

wherein

R₄ is H or alkyl, preferably -methyl;

R₅ is H or alkyl, preferably -methyl or t-butyl;

R₆ is H, alkyl, or CH₂(CO)OCH₃;

R₇ is H, alkyl or halogen;

R₈ is H or alkyl, preferably t-butyl;

R₉ is H or alkyl, preferably methyl or t-butyl;

R₁₀ is H or alkyl, preferably methyl or t-butyl;

R₁₁ and R₁₂ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, or halogen;

A straight line overlayed by a wavy line denotes the covalent bond of the respective residue to the Formula (I).

The alkyl groups as described above each independently may be selected from the group consisting of methyl, ethyl, propyl (e.g., n-propyl and i-propyl), and butyl (e.g., n-butyl, s-butyl, and t-butyl).

The carbon atoms marked with “*” each independently may have D or L stereoisomeric configuration. In some embodiments, all of the carbon atoms marked with “*” have D stereoisomeric configuration.

In some embodiments, R₂ and R₃ are identical residues. For example, R₂ and R₃ are H, bis-POM, or bis-AM.

In some embodiments, R2 and R3 are H.

In some embodiments, R₂ and R₃ are ethyl or phenyl.

In some embodiments, R₂ is ethyl and R₃ is phenyl, or R₃ is ethyl and R₂ is phenyl.

In some embodiments, R₂ and R₃ are both

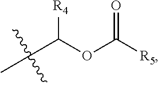

wherein R₄ is H, methyl; R₅ is alkyl (e.g., methyl or t-butyl). In prefered embodiments, R₄ is H and R₅ is methyl. Hence, R₂, R₃ may both be acetoxymethyl (AM). In some other preferred embodiments, R₄ is H and R₅ is t-butyl. Hence, R₂, R₃ may both be pivaloyloxymethyl (POM).

In some embodiments, R₂ and R₃ are both

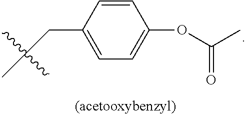 [SMILES: *Cc1ccc(OC(C)=O)cc1]

In some embodiments, R₂ and R₃ are both

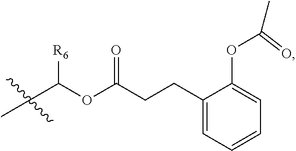

wherein R₆ is H, alkyl, or CH₂(CO)OCH₃.

In some embodiments, R₂ and R₃ jointly form

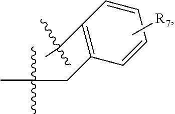

wherein R₇ is alkyl or halogen.

In some embodiments, R₂ and R₃ jointly form

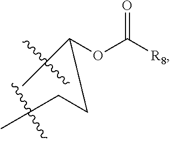

wherein R₈ is t-butyl.

In some embodiments, R₂ and R₃ are S-[(2-hydroxyethyl)sulfidyl]-2-thioethyl (DTE), or

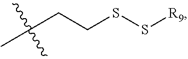

wherein R₉ is alkyl, preferably methyl, ethyl, propyl, or butyl (e.g., t-butyl).

In some embodiments, R₂ and R₃ are S-acyl-2-thioethyl (SATE), or

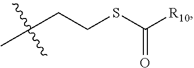

wherein R₁₀ is alkyl, preferably methyl, ethyl, propyl, or butyl (e.g., t-butyl).

### Derivatives of 4′-Phosphopantothenoyl-L-Cysteine:

In yet another aspect, active derivatives of 4′-phopshopanthetheine also include 4′-phosphopantothenoyl-L-cysteine and its derivatives.

### Derivatives of Dephospho-CoA:

In yet another aspect, active derivatives of 4′-phopshopanthetheine also include dephospho-CoA and its derivatives.

### Methods of the Application

In one aspect, the present application relates to a method of treating a diseased subject having a disease associated with insufficient pantothenate kinase activity, comprising administering to the diseased subject an effective amount of an active derivative of 4′-phosphopantetheine (e.g., a compound of Formula (I), a pharmaceutically acceptable salt, or a solvate thereof).

In another aspect, the present application relates to a method of treating a diseased subject having a disease associated with an inhibition of one or more pantothenate kinases (e.g., wild type pantothenate kinases) by the over-accumulation of one or more CoA species (e.g., acyl-CoA species), comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a Coenzyme A sequestration, toxicity or redistribution (CASTOR) disease, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease associated with decreased concentrations of CoA and/or acetyl-CoA, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of modifying or increasing concentrations of CoA and/or acetyl-CoA, comprising administering to a subject in need thereof an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease associated with impaired or inhibited degradation of one or more acyl-CoA species, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease associated with accumulation of one or more fatty acids, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease associated with impaired or inhibited degradation of one or more fatty acids, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease associated with abnormal CoA homeostasis, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

In yet another aspect, the present application relates to a method of treating a diseased subject having a disease selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, and PLA2G6-associated neurodegeneration.

In yet another aspect, the present application relates to a method of treating a diseased subject having a disease selected from the group consisting of glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease selected from the group consisting of medium chain acyl-CoA dehydrogenase deficiency, short chain acyl-CoA dehydrogenase deficiency, very long chain acyl-CoA dehydrogenase deficiency, and D-bifunctional protein deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a medium chain acyl-CoA dehydrogenase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a short chain acyl-CoA dehydrogenase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a very long chain acyl-CoA dehydrogenase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a D-bifunctional protein deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a disease is selected from the group consisting of Glutaric acidemia type 1, methylmalonic academia, propionyl-CoA carboxylase deficiency, propionic academia, 3-methylcrotonyl carboxylase deficiency, and isovaleryl-CoA dehydrogenase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having Glutaric acidemia type 1, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having methylmalonic academia, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a propionyl-CoA carboxylase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having propionic academia, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a 3-methylcrotonyl carboxylase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of treating a diseased subject having a isovaleryl-CoA dehydrogenase deficiency, comprising administering to the diseased subject an effective amount of an active derivative of 4′-phosphopantetheine (e.g., a compound of Formula (I), a pharmaceutically acceptable salt, or a solvate thereof).

In yet another aspect, the present application relates to a method of preparing a pharmaceutical composition comprising one or more active derivatives of 4′-phosphopantetheine (e.g., a compound of Formula (I), a pharmaceutically acceptable salt, or a solvate thereof).

In yet another aspect, the present application relates to use of an active derivative of 4′-phosphopantetheine (e.g., a compound of Formula (I), a pharmaceutically acceptable salt, or a solvate thereof) in manufacturing a pharmaceutical composition for treating a diseased subject having a CASTOR disease.

In yet another aspect, the present application relates to use of an active derivative of 4′-phosphopantetheine (e.g., a compound of Formula (I), a pharmaceutically acceptable salt, or a solvate thereof) in treating a diseased subject having a CASTOR disease.

In yet another aspect of the present application relates to use of a pharmaceutical composition comprising one or more of active derivatives of 4′-phosphopantetheine (e.g., a compound of Formula (I), a pharmaceutically acceptable salt, or a solvate thereof) in treating a diseased subject having a CASTOR disease.

In some embodiments, the diseased subject has one or more deficient, defective, and/or absent pantothenate kinases.

In some embodiments, the diseased subject has one or more aberrantly expressed pantothenate kinases.

In some embodiments, the diseased subject does not have one or more deficient, defective, and/or absent pantothenate kinases.

In some embodiments, the diseased subject does not have one or more aberrantly expressed pantothenate kinases.

### Synthesis of Active Derivatives of 4′-Phosphopantetheine

A compound of the present application may be made by a variety of methods, including standard chemistry. The synthetic processes of the application can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester, or prodrug thereof. Suitable synthetic routes are depicted in the schemes below.

A compound of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of a compound of the present application.

A compound disclosed herein may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of a compound disclosed herein.

Those skilled in the art will recognize if a stereocenter exists in a compound disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

All the abbreviations used in this application are found in “Protective Groups in Organic Synthesis” by John Wiley & Sons, Inc, or the MERCK INDEX by MERCK & Co., Inc, or other chemistry books or chemicals catalogs by chemicals vendor such as Aldrich, or according to usage know in the art.

A compound of the present application can be prepared in a number of ways well known to those skilled in the art of organic synthesis. By way of example, a compound of the present application can be synthesized using the methods described below, together with synthetic methods known in the art of synthetic organic chemistry, or variations thereon as appreciated by those skilled in the art. Preferred methods include but are not limited to those methods described below.

In one aspect, the present application relates to a method of synthesizing one or more active derivatives of 4′-phosphopantetheine (e.g., a compound of Formula (I), a pharmaceutically acceptable salt, or a solvate thereof), comprising the steps of:
i) chemically treating pantothenic acid with S-tritylcysteamine to form S-tritylpantetheine;
ii) chemically treating S-tritylpantetheine with dibenzylchlorophosphate to form S-trityl-4′-dibenzylphosphopantetheine; and
iii) chemically treating S-trityl-4′-dibenzylphosphopantetheine to form 4′-phosphopantetheine.

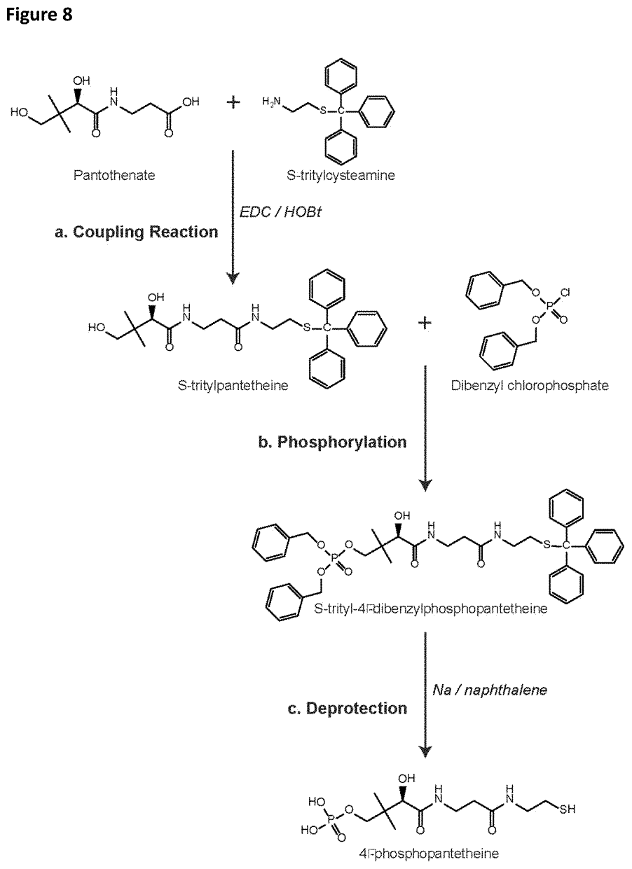

In some embodiments, an active derivative of 4′-phosphopantetheine is synthesized by following the steps outlined in FIG. 8. Starting materials are either commercially available or made by known procedures in the reported literature or as illustrated.

A mixture of enantiomers, diastereomers, and/or cis/trans isomers resulting from the methods described above can be separated into their single components by chiral salt technique, chromatography using normal phase, or reverse phase or chiral column, depending on the nature of the separation.

It should be understood that, for synthetic purposes, the compounds in the methods described above are mere representatives with elected substituents to illustrate the general synthetic methodology of active derivative of 4′-phosphopantetheine disclosed herein.

### Biological Assays

An active derivative of 4′-phosphopantetheine disclosed herein can be tested for its activity with various biological assays. Suitable assays include, but are not limited to, cell culture (e.g., Drosophila S2 cell culture), cell treatment (e.g., RNA Interference, cell treatment with an active derivative of 4′-phosphopantetheine, or cell treatment with Haloperidol (HoPan)), cell staining (e.g., Immunofluorescence Staining), gene knock-down (e.g., knock-down of COASY by siRNA in mammalian HEK293 cells), western blot analysis, RNA Isolation, Quantitative Real-Time PCR, Parallel Artificial Membrane Permeability Assay (PAMPA), and animal (e.g., mice) injection study.

### Coenzyme A Sequestration, Toxicity or Redistribution (CASTOR) Diseases

In one aspect, a CASTOR disease may be associated with the inhibition of one or more pantothenate kinases (e.g., wild type pantothenate kinases), and such inhibition may be caused by accumulation of one or more inhibitors of pantothenate kinases. The CASTOR disease may be associated the inhibition of one or more pantothenate kinases by the over-accumulation of one or more CoA species (e.g., acyl-CoA species) in a disease state. In some embodiments, over-accumulation of one or more CoA species (e.g., acyl-CoA species) in CASTOR diseases can lead to decrease in intracellular levels of CoA and/or acetyl-CoA, two key molecules of cellular metabolism. Decrease in the concentrations of CoA and acetyl-CoA can therefore negatively affect numerous metabolic reactions in the cells and lead to a variety of disease conditions.

In some embodiments, the CASTOR disease is not associated with deficiency, defectiveness, and/or absence of one or more pantothenate kinases.

In some embodiments, the CASTOR disease is not associated with aberrant expression of one or more pantothenate kinases.

In some embodiments, the CASTOR disease is not a pantothenate kinase-associated neurodegeneration (PLAN) disease.

In another aspect, a CASTOR disease may be characterized by, or associated with, accumulation of one or more acyl Coenzyme A (acyl-CoA) species in a diseased subject to amounts greater than that of a normal healthy subject not having the disease. The accumulation may be caused by impaired or inhibited degradation of one or more acyl-CoA species in the diseased subject.

In some embodiments, the acyl-CoA species is acetoacetyl-CoA, acetyl-CoA, butyryl-CoA, cinnamoyl-CoA, coumaroyl-CoA, crotonyl-CoA, glutaconyl-CoA, glutaryl-CoA, 3-hydroxy-3-methylglutaryl-CoA (HMG-CoA), beta-hydroxy beta-methylbutyryl-CoA (HMB-CoA), 3-hydroxybutyryl-CoA, 3-hydroxyisobutyryl-CoA, isovaleryl-CoA, malonyl-CoA, methacrylyl-CoA, 2-methylacetoacetyl-CoA, 2-methylbutyryl-CoA, methylcrotonyl-CoA, 3-methylglutaconyl-CoA, methylmalonyl-CoA, octanoyl-CoA, 3-oxoacyl-CoA, palmitoyl-CoA, phytanoyl-CoA, propionyl-CoA, stearoyl-CoA, succinyl-CoA, or tiglyl-CoA. In some embodiments, the acyl-CoA species is acetyl-CoA, a fatty acyl-CoA (e.g., propionyl-CoA, butyryl-CoA, myristoyl-CoA, or crotonyl-CoA), or its derivatives (e.g., 2-methyl-acetoacetyl-CoA, 2-methyl-3-OH-butyryl-CoA, tiglyl-CoA, 2-methylbutyryl-CoA, 3-methylcrotonyl-CoA, 3-methylglutaconyl-CoA, 3-OH-3-methylglutaryl-CoA, malonyl-CoA, methylmalonyl-CoA, or succinyl-CoA).

In certain embodiments, the acyl-CoA species is not acetyl-CoA.

In another aspect, a CASTOR disease may be characterized by, or associated with, accumulation of one or more fatty acids in a diseased subject to amounts greater than that of a normal healthy subject not having the disease. The accumulation may be caused by impaired or inhibited degradation of one or more fatty acids in the diseased subject.

In some embodiments, the fatty acid is a long chain fatty acid, a medium chain fatty acid, or a short chain fatty acid. For example, the fatty acid may be propionic acid (propanoic acid), butyric acid (butanoic acid), valeric acid (pentanoic acid), caproic acid (hexanoic acid), enanthic acid (heptanoic acid), caprylic acid (octanoic acid), pelargonic acid (nonanoic acid), capric acid (decanoic acid), undecylic acid (undecanoic acid), lauric acid (dodecanoic acid), tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), heneicosylic acid (heneicosanoic acid), behenic acid (docosanoic acid), tricosylic acid (tricosanoic acid), lignoceric acid (tetracosanoic acid), pentacosylic acid (pentacosanoic acid), cerotic acid (hexacosanoic acid), heptacosylic acid (heptacosanoic acid), montanic acid (octacosanoic acid), nonacosylic acid (nonacosanoic acid), melissic acid (triacontanoic acid), henatriacontylic acid (henatriacontanoic acid), lacceroic acid (dotriacontanoic acid), psyllic acid (tritriacontanoic acid), geddic acid (tetratriacontanoic acid), ceroplastic acid (pentatriacontanoic acid), hexatriacontylic acid (hexatriacontanoic acid), heptatriacontanoic acid (heptatriacontanoic acid), or octatriacontanoic acid (octatriacontanoic acid). For another example, the fatty acid may be α-linolenic acid, stearidonic acid, eicosapentaenoic acid, docosahexaenoic acid, linoleic, γ-linolenic acid, dihomo-γ-linolenic acid, arachidonic acid, docosatetraenoic acid, palmitoleic acid, ω-7 vaccenic acid, paullinic acid, oleic acid, elaidic acid, gondoic acid, erucic acid, nervonic acid, or mead acid.

In yet another aspect, a CASTOR disease may be characterized by, or associated with, decrease of free CoA and/or acetyl-CoA in a diseased subject to amounts lower than that of a normal healthy subject not having the disease. The decrease may be caused by accumulation of one or more acyl-CoA species in the diseased subject to amounts greater than that of a normal healthy subject not having the disease.

In yet another aspect, a CASTOR disease may be selected from the group consisting of: medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/ multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I and ethylmalonic encephalopathy.

In yet another aspect, a CASTOR disease may be selected from the group consisting of: medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, and 3-methylglutaconic aciduria and PLA2G6-associated neurodegeneration (Mitchell GA et al, Mol Genet Metab 94:4-15 (2008)).

In yet another aspect, a CASTOR disease may be selected from the group consisting of: glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency /Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA: amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I and ethylmalonic encephalopathy.

In yet another aspect, a CASTOR disease may be acquired CASTOR diseases. The acquired CASTOR diseases may be caused by intake of xenobiotic organic acids due to acute or chronic poisoning, or medical treatments or medical conditions which result in accumulation of fatty acids in the cytosol or mitochondria of cells. Examples of acquired CASTOR diseases include: Reye syndrome and Reye-like syndrome, poisoning by benzoic acid, poisoning by aspirin, poisoning by acetylsalicylic acid, poisoning by salicylic acid, poisoning by valproic acid, Ischemia, reperfusion injury, non-alcoholic fatty liver disease.

CASTOR diseases are frequently related to episodic acute metabolic decompensations, which can be triggered by stress, prolonged fasting, exercise, infection or illness and require urgent medical attention otherwise coma and death may occur in a high proportion of patients. This application thus relates to treatment of these acute metabolic decompensations.

Treatment of CASTOR diseases with active derivatives of 4′-phosphopantetheine (e.g., 4′-phosphopantetheine or S-acetyl-4′-phosphopantetheine) has a number of advantages. Namely, as described in detail in the Examples below, active derivatives of 4′-phosphopantetheine may increase intracellular CoA levels through a pantothenate kinase-independent mechanism. In some embodiments, an active derivatives of 4′-phosphopantetheine (e.g., 4′-phosphopantetheine or S-acetyl-4′-phosphopantetheine) is serum stable and/or readily synthesized.

In some embodiments, the CASTOR disease is selected from the group consisting of: medium chain acyl-CoA dehydrogenase deficiency, short chain acyl-CoA dehydrogenase deficiency, very long chain acyl-CoA dehydrogenase deficiency and D-bifunctional protein deficiency.

In some embodiments, the CASTOR disease is medium chain acyl-CoA dehydrogenase deficiency.

In some embodiments, the CASTOR disease is short chain acyl-CoA dehydrogenase deficiency.

In some embodiments, the CASTOR disease is very long chain acyl-CoA dehydrogenase deficiency.

In some embodiments, the CASTOR disease is D-bifunctional protein deficiency.

In some embodiments, the CASTOR disease is selected from the group consisting of: Glutaric acidemia type 1, methylmalonic academia, propionyl-CoA carboxylase deficiency, propionic academia, 3-methylcrotonyl carboxylase deficiency and isovaleryl-CoA dehydrogenase deficiency.

In some embodiments, the CASTOR disease is Glutaric acidemia type 1.

In some embodiments, the CASTOR disease is methylmalonic academia.

In some embodiments, the CASTOR disease is propionyl-CoA carboxylase deficiency.

In some embodiments, the CASTOR disease is propionic academia.

In some embodiments, the CASTOR disease is 3-methylcrotonyl carboxylase deficiency.

In some embodiments, the CASTOR disease is isovaleryl-CoA dehydrogenase deficiency.

In some embodiments, the CASTOR disease is Reye syndrome.

### Pharmaceutical Compositions

The compounds disclosed herein can be included in pharmaceutical compositions (including therapeutic and prophylactic formulations), typically combined together with one or more pharmaceutically acceptable vehicles or carriers and, optionally, other therapeutic ingredients.

Such pharmaceutical compositions can be formulated for administration to subjects by a variety of mucosal administration modes, including by oral, rectal, intranasal, intrapulmonary, intravitrial, or transdermal delivery, or by topical delivery to other surfaces including the eye. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other examples, the compound can be administered ex vivo by direct exposure to cells, tissues or organs originating from a subject.

To formulate the pharmaceutical compositions, the compound can be combined with various pharmaceutically acceptable additives, as well as a base or carrier useful in the dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween®80), solubility enhancing agents (for example, cyclodextrins and derivatives thereof), stabilizers (for example, serum albumin), and reducing agents (for example, glutathione) can be included.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7. The compound can be dispersed in a carrier, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof.

Alternatively or additionally, synthetic fatty acid esters such as polyglycerin fatty acid esters, sucrose fatty acid esters and the like can be employed as carriers. Hydrophilic polymers and other vehicles can be used alone or in combination, and enhanced structural integrity can be imparted to the vehicle by partial crystallization, ionic bonding, cross-linking and the like. The carrier can be provided in a variety of forms, including fluid or viscous solutions, gels, pastes, powders, microspheres, and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanoparticles prepared from a suitable polymer, for example, 5 isobutyl 2-cyanoacrylate (see, for example, Michael et al., I Pharmacy Pharmacol. 43, 1-5, 1991), and dispersed in a biocompatible dispersing medium, which yields sustained delivery and biological activity over a protracted time. Alternatively, the compound may be combined with a mesoporous silica nanoparticle including a mesoporous silica nanoparticle complex with one or more polymers conjugated to its outer surface.

The pharmaceutical compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like. Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having 49hydrolysable ester linkages. A number of these are known in the art to be biodegradable and to lead to degradation products having no or low toxicity. Exemplary polymers include polyglycolic acids and polylactic acids, poly(DL-lactic acidco-glycolic acid), poly(D-lactic acid-co-glycolic acid), and poly(L-lactic acid-coglycolic acid). Other useful biodegradable or bioerodable polymers include, but are not limited to, such polymers as poly(epsilon-caprolactone), poly(epsilon-aprolactone-CO-lactic acid), poly(epsilon-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In one aspect, the present application relates to a pharmaceutical compositions for treating a diseased subject having one or more of the diseases described herein.

In another aspect, the present application relates to a pharmaceutical compositions for use in one or more of the methods described herein.

In yet another aspect, of the present application features a pharmaceutical composition for use in treating a diseased subject having a disease associated with insufficient pantothenate kinase enzyme activity. The insufficient pantothenate kinase activity may result from inhibition of pantothenate kinase by amounts of one or more CoA species greater than that of a healthy subject not having the disease (e.g., CASTOR diseases).

In yet another aspect, the present application features a pharmaceutical composition for use in the treatment of a diseased subject having a disease associated with impaired CoA homeostasis.

In yet another aspect, the present application features a pharmaceutical composition for use in the treatment of a diseased subject having a disease associated with one or more defects in metabolic enzymes that are involved in maintenance of normal levels of CoA species.

In yet another aspect, the present application features a pharmaceutical composition for use in the treatment of a diseased subject having a disease associated with one or more genetic defects affecting the activity of an enzyme having catalytic activity on a CoA species.

In some embodiments, the pharmaceutical composition comprises an effective amount of 4′-phosphopantetheine or a compound of Formula (I), a pharmaceutically acceptable salt thereof, or a solvate thereof.

In some embodiments, the pharmaceutical composition comprises an effective amount of 4′-phosphopantetheine, a pharmaceutically acceptable salt thereof, or a solvate thereof.

In some embodiments, the pharmaceutical composition comprises an effective amount of a compound of Formula (I), a pharmaceutically acceptable salt thereof, or a solvate thereof.

In some embodiments, the pharmaceutical composition comprises an effective amount of a compound of Formula (Ia), a pharmaceutically acceptable salt thereof, or a solvate thereof.

In some embodiments, the pharmaceutical composition comprises an effective amount of S-acyl-4′-phosphopantetheine, a pharmaceutically acceptable salt thereof, or a solvate thereof.

In some embodiments, the pharmaceutical composition comprises an effective amount of S-propionyl-4′-phosphopantetheine, a pharmaceutically acceptable salt thereof, or a solvate thereof.

In some embodiments, the pharmaceutical composition comprises an effective amount of S-acetyl-4′-phosphopantetheine, a pharmaceutically acceptable salt thereof, or a solvate thereof.

In some embodiments, the pharmaceutical composition comprises an effective amount of 4′-phosphopantothenate or an active derivative thereof, a pharmaceutically acceptable salt thereof, or a solvate thereof.

In some embodiments, the pharmaceutical composition comprises an effective amount of 4′-phosphopantothenate or a compound of Formula (II), a pharmaceutically acceptable salt thereof, or a solvate thereof.

In some embodiments, the pharmaceutical composition comprises an effective amount of 4′-phosphopantothenate, a pharmaceutically acceptable salt thereof, or a solvate thereof.

In some embodiments, the pharmaceutical composition comprises an effective amount of a compound of Formula (II), a pharmaceutically acceptable salt thereof, or a solvate thereof.

In some embodiments, the pharmaceutical composition is formulated for oral administration, topical administration, sublingual administration, inhalation, or injection (e.g., intravenous administration, intramuscular administration, and subcutaneous administration).

### Pharmaceutical Kits

In one aspect, the present application relates pharmaceutical kits comprising a therapeutically effective amount of a pharmaceutical composition including (a) an active derivative of 4′-phosphopantetheine and/or (b) one or more active derivatives of 4′-phosphopantetheine, in one or more sterile containers. Sterilization of the container can be carried out using conventional sterilization methodology well known to those skilled in the art. The one or more active derivatives of 4′-phosphopantetheine can be in the same sterile container or in separate sterile containers. The sterile containers or materials can include separate containers, or one or more multi-part containers, as desired. The one or more active derivatives of 4′-phosphopantetheine can be separate, or physically combined into a single dosage form or unit. The kits can further include one or more of various conventional pharmaceutical kit components (e.g., one or more pharmaceutically acceptable carriers, additional vials for mixing the components), as should be readily apparent to those skilled in the art. Instructions, either as inserts or as labels, indicating quantities of the components to be administered, guidelines for administration, and/or guidelines for mixing the components, can also be included in the kit.

### Definitions

Listed below are definitions of various terms used to describe present application. These definitions apply to the terms as they are used throughout this specification and claims, unless otherwise limited in specific instances, either individually or as part of a larger group.

The term “alkyl,” as used herein, refers to a straight or branched hydrocarbon chain radical consisting of carbon and hydrogen atoms, containing no saturation, having one to eight carbon atoms, and which is attached to the rest of the molecule by a single bond. Examples of alkyl radicals include, but are not limited to methyl, ethyl, n-propyl, i-propyl, n-butyl, t-butyl, and n-pentyl radicals. Alkyl radicals may be optionally substituted by one or more substituents. Examples of the substituents include, but are not limited to, aryl, halo, hydroxy, alkoxy, carboxy, cyano, carbonyl, acyl, alkoxycarbonyl, amino, nitro, mercapto, and alkylthio radicals.

The term “aralkyl,” as used herein, refers to an alkyl radical substituted with one or more aryl radicals. Example of alrakyl radicals include, but are not limited to, benzyl and phenethyl radicals.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “cycloalkyl,” as used herein, refers to a stable 3- to 10-membered monocyclic or bicyclic radical which is saturated or partially saturated, and which consist solely of carbon and hydrogen atoms, such as cyclohexyl or adamantyl. Unless otherwise defined, the term “cycloalkyl” is meant to include cycloalkyl radicals which are optionally substituted by one or more substituents such as alkyl, halo, hydroxy, amino, cyano, nitro, alkoxy, carboxy, alkoxycarbonyl.

The term “aryl,” as used herein, refers to single or multiple ring radicals, including multiple ring radicals that contain separate and/or fused aryl groups. Typical aryl groups contain from 1 to 3 separated or fused rings and from 6 to about 18 carbon ring atoms. Example of aryl radicals include, but are not limited to, phenyl, naphthyl, indenyl, fenanthryl, and anthracyl radicals. The aryl radical may be optionally substituted by one or more substituents, such as hydroxy, mercapto, halo, alkyl, phenyl, alkoxy, haloalkyl, nitro, cyano, dialkylamino, aminoalkyl, acyl, and alkoxycarbonyl.

The term “heterocyclyl,” as used herein, refers to a stable 3 to 15 membered ring radical that consists of carbon atoms and from one to five heteroatoms selected from the group consisting of nitrogen, oxygen, and sulfur, preferably a 4-to 8-membered ring with one or more heteroatoms, more preferably a 5-or 6-membered ring with one or more heteroatoms. The heterocyclyl radicals may be aromatic or non-aromatic. The heterocycle may be a monocyclic, bicyclic, or tricyclic ring system, which may include fused ring systems; and the nitrogen, carbon, or sulfur atoms in the heterocyclyl radical may be optionally oxidised; the nitrogen atom may be optionally quaternized; and the heterocyclyl radicals may be partially or fully saturated or aromatic. Examples of heterocyclyl radicals include, but are not limited to, azepines, benzimidazole, benzothiazole, furan, isothiazole, imidazole, indole, piperidine, piperazine, purine, quinoline, thiadiazole, tetrahydrofuran, coumarine, morpholine; pyrrole, pyrazole, oxazole, isoxazole, triazole, and imidazole.

The term “alkoxy,” as used herein, refers to a radical of —O-alkyl, where wherein alkyl is an alkyl radical as defined above.

The term “substituted,” as used herein, refers to the replacement of hydrogen in a given structure with the radical of a suitable group. Examples of the suitable groups include, but are not limited to, halogen (e.g., fluoro, chloro, bromo, and iodo), cyano, hydroxyl, nitro, azido, alkanoyl (e.g., C1-6 alkanoyl, such as acyl), carboxamido, alkyl (e.g., alkyl radicals having 1 to 12 carbon atoms or 1 to 6 carbon atoms and, more preferably, 1 to 3 carbon atoms), alkenyl (e.g., alkenyl radicals having 2 to 12 carbon atoms or 2 to 6 carbon atoms), alkynyl (e.g., alkynyl radicals having 2 to 12 carbon atoms or 2 to 6 carbon atoms), alkoxy (e.g., alkoxy radicals having one or more oxygen linkages and from 1 to about 12 carbon atoms or 1 to about 6 carbon atoms), aryloxy (e.g., phenoxy), alkylthio (e.g., radicals having one or more thioether linkages and from 1 to about 12 carbon atoms or from 1 to about 6 carbon atoms), alkylsulfinyl (e.g., radicals having one or more sulfinyl linkages and from 1 to about 12 carbon atoms or from 1 to about 6 carbon atoms), alkylsulfonyl (e.g., radicals having one or more sulfonyl linkages and from 1 to about 12 carbon atoms or from 1 to about 6 carbon atoms), aminoalkyl (e.g., radicals having one or more N atoms and from 1 to about 12 carbon atoms or from 1 to about 6 carbon atoms); and carbocylic aryl (e.g., carbocyclic aryl radicals having 6 or more carbons, particularly phenyl or naphthyl and aralkyl such as benzyl). Unless otherwise indicated, an optionally substituted group may have a substituent at each substitutable position of the group, and each substitution is independent of the other.

The term “pharmaceutically acceptable salts or solvates,” as used herein, refers to any pharmaceutically acceptable salt, solvate, or any other compound which, upon administration to the recipient is capable of providing (directly or indirectly) a compound as described herein. However, it will be appreciated that non-pharmaceutically acceptable salts also fall within the scope of the application since those may be useful in the preparation of pharmaceutically acceptable salts. The preparation of salts, prodrugs and derivatives can be carried out by methods known in the art. For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic aminoacids salts.

The terms “administration”, “administer”, or “administering,” as used herein, refer to providing or giving a subject an agent, such as a pharmaceutical composition by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

The term “effective amount,” as used herein, refers to an amount of agent (e.g., 4′-phosphopantetheine or an active derivative thereof) that is sufficient to generate a desired response in a subject (e.g., increasing intracellular CoA in a cell or treating one or more of the signs or symptoms of a CASTOR disease or abnormal CoA homeostasis). An effective amount can be a prophylactically effective amount including an amount that prevents one or more signs or symptoms of a disease from developing.

The terms “inhibit”, “inhibiting”, “inhibition”, “treat”, “treating” or “treatment”, as used herein, refer to slowing, stopping, or reversing the development of a disease (e.g., a CASTOR disease or a disease associated with abnormal CoA homeostasis). A prophylactic treatment is administered to a subject that does not exhibit signs or symptoms of a disease for the purpose of decreasing the risk of developing the disease. A therapeutic treatment is administered after the development of significant signs or symptoms of the disease.

The term “subject,” as used herein, refers to a living multicellular vertebrate organism including, for example, mammals and birds. Mammals include both human and non-human mammals such as mice. In some examples, the subject is a patient such as a patient with a CASTOR disease or patient with a disease associated with abnormal CoA homeostasis.

The term “active derivative of 4′-phosphopantetheine,” as used herein, refers to 4′-phosphopantetheine and derivatives thereof.

The disclosure having been described, the following examples are offered by way of illustration and not limitation.

### EXAMPLES

As further described herein, in flies, and in human and mouse serum, CoA is rapidly hydrolyzed by ecto-nucleotide-pyrophosphatases to 4′-phosphopantetheine, a biologically stable molecule that is able to translocate through membranes via passive diffusion. Inside the cell, 4′-phosphopantetheine is enzymatically converted back to CoA by the bifunctional enzyme CoA synthase.

In CoA-deprived flies, worms and human cells, CoA provided via the food or media rescues cell growth, decreased protein acetylation, abnormal locomotor skills, developmental arrest, sterility, and decreased lifespan. The findings disclosed herein answer long-standing questions in fundamental cell biology and have major implications for understanding CoA-related diseases and for developing new CoA targeting strategies to treat parasites and microbial infections.

Identification of CoA-acquiring mechanisms is of importance for treatment of neurodegenerative disorders caused by defects in the CoA biosynthesis pathway. As described herein, it is demonstrated that extracellular CoA levels influence intracellular CoA levels both in vitro and in vivo. Further, it is disclosed that CoA is not a biologically stable molecule and that cells do not take up CoA directly.

### Synthetic Methods

4′-Phosphopantetheine (PPanSH) Synthesis Protocol: 4′-Phosphopantetheine (PPanSH) was synthesized in a three-step procedure as described below (a/b/c) (FIG. 8). In the first step, commercially available pantothenic acid was coupled with synthesized S-tritylcysteamine. The obtained S-tritylpantetheine was then phosphorylated with freshly prepared dibenzylchlorophosphate. Finally, removal of benzyl groups provided 4′-phosphopantetheine. D-Pantothenic acid was prepared from its hemicalcium salt (Aldrich, ≥99.0%) by reacting with oxalic acid in distilled water. The precipitated calcium oxalate was filtered off, while the protonated form of D-pantothenic acid was obtained by evaporation of water. S-tritylcysteamine was synthesized from cysteamine hydrochloride and trityl chloride (Mandel A L et al, Organic Letters 6, 4801-48 (2004). Dibenzylchlorophosphate was prepared by reacting dibenzylphosphite with N chlorosuccinimide (Itoh K et al, Organic Letters 9, 879-882 (2007)) in toluene as a solvent. All other chemicals were obtained from commercial sources and used without further purification; cysteamine hydrochloride (Aldrich, ≥98.0%), trityl chloride (Aldrich, 97.0%), N-(3-dimethylaminopropyl)-N′-ethylcarbodiimide (EDC) (Aldrich, ≥97.0%), 1-hydroxybenzotriazole hydrate (HOBt) (Aldrich, ≥97.0%), dibenzylphosphite (Aldrich, technical grade), N-chlorosuccinimide (Aldrich, 98%). Column chromatography was carried out using Silica gel 60 A, 60-100 mesh (Aldrich). Cation exchange chromatography was performed on DOWEX 50WX2, hydrogen form, 100-200 mesh (Aldrich). ¹H and ¹³C NMR were recorded at 25° C. with Varian Unity Inova 300 MHz spectrometer (300 MHz/75 MHz). The chemical shifts (δ) are reported in ppm units relative to TMS as an internal standard where spectra recorded in CDCl3 or relative to residual solvent signal when D2O was used. High-resolution mass spectra were obtained on AutospecQ mass spectrometer with negative electrospray ionization.

Coupling reaction—synthesis of S-tritylpantetheine: In dried acetonitrile (100 ml) the following were prepared separately: (A) D-pantothenic acid (2.19 g, 10.0 mmol), (B) S tritylcysteamine (3.19 g, 10.0 mmol) and (C) N-(3-dimethylaminopropyl)-N′-ethylcarbodiimide (EDC) (1.55 g, 10.0 mmol) together with 1-hydroxybenzotriazole hydrate (HOBt) (1.35 g, 10.0 mmol). After A, B and C were mixed together, triethylamine (10.4 ml, 75 mmol) was added. The mixture was stirred at room temperature for 24 h and quenched with addition of water (400 ml). The product was extracted with diethyl ether (3×250 ml). The combined organic phases were washed with 1 M hydrochloric acid, saturated aqueous solution of NaHCO3 (500 ml), and brine (500 ml). The organic layer was dried over sodium sulfate and concentrated in vacuum S-trityl-pantetheine (3.53 g, 68%) was synthesized as pale-yellow crystals. 1H NMR (300 MHz,

CDCl3) δ 0.85 (s, 3H), 0.92 (s, 3H), 2.29 (app t, J=6.2 Hz, 2H), 2.38 (td, J=2.3, 6.6, 6.8 Hz, 2H), 3.03 (m, 2H), 3.38-3.49 (m, 4H), 3.92 (s, 1H), 6.20 (t, J=5.7 Hz, 1H, NH), 7.17-7.29 (m, 10 H), 7.36-7.45 (m, 5H).

Phosphorylation—Synthesis of S-trityl-4′-dibenzylphosphopantetheine: Dibenzylchlorophosphate was freshly prepared by allowing a reaction of dibenzylphosphite (2.16 g, 8.24 mmol) with N-chlorosuccinimide (1.21 g, 9.06 mmol) in toluene (40 ml) at room temperature for 2 h. The mixture was filtered and the filtrate was evaporated under vacuum and added to a solution of S-tritylpantetheine (2.86 g, 5.49 mmol), diisopropylethylamine (3.06 ml), 4-dimethylaminopyridine (0.067 g, 0.55 mmol) in dry acetonitrile (50 ml). The mixture was stirred for 2 h at room temperature.

Acetonitrile was removed under vacuum. Products were extracted into organic phase in dichloromethane (3×100 ml)—aqueous NaHCO₃ (100 ml) system. The organic extracts were washed with water (100 ml), and dried over Na₂SO₄. Evaporation of solvent gave a crude S-trityl-4′-dibenzylphosphopantetheine as a dark brown oil (4.69 g), which was further purified by flash chromatography (SiO₂, EtOAc, MeOH) to give a semicrystaline pale yellow product (0.640 g, 0.82 mmol). The yield of the synthesis and purification of S-trityl-4′-dibenzylphosphopantetheine is 15%. 1H NMR (300 MHz, CDCl3) δ 0.75 (s, 3H), 1.03 (s, 3H), 2.32 (app t, J=6.1 Hz, 2H), 2.4 (app t, J=6.5 Hz, 2H), 3.06 (app q, J=6.3 Hz, 2H), 3.47 (app q, 6.0 Hz, 2H), 3.60 (dd, J=9.9, 7.3 Hz 1H), 3.85 (s, 1H), 4.00 (dd, J=9.9, 7.0 Hz, 1H), 4.99-5.04 (m, 4H), 5.80 (t, J=5.5 Hz, 1H, NH), 7.16-7.32 (m, 20H), 7.38-7.40 (m, 5H).

Deprotection—Synthesis of 4′-phosphopantetheine: Naphthalene (12.9 g, 100.6 mmol) dissolved in tetrahydrofuran (70 ml) was added to sodium metal (Na) (2.21 g, 96.1 mmol) in tetrahydrofuran (50 mL). After 2 h the solution was cooled to −(35±5)° C. and S-trityl-4′-dibenzylphosphopantetheine (1.85 g, 2.37 mmol) dissolved in tetrahydrofuran (70 ml) was slowly added. The mixture was stirred for 2 h while maintaining the temperature below −30° C. The reaction was quenched by addition of water (100 ml) and then dichloromethane (200 ml) was added. Phases were separated and the aqueous phase (together 500 ml) was washed with dichloromethane (200 ml) and diethyl ether (3×200 ml), concentrated under vacuum and passed through the cation exchange column (DOWEX 50WX2, 200 g). Fractions were analyzed by LCMS and those containing the product were pooled and concentrated under vacuum. 4′-phosphopantetheine was precipitated with addition of Ca(OH)₂ as a calcium salt (332 mg, 0.838 mmol, 35%). The structure of the product was confirmed by comparison of NMR data with the literature and by HRMS. 1H NMR (300 MHz, D2O) δ 0.86 (s, 3H), 1.08 (s, 3H), 2.54 (app t, J=6.3 Hz, 2H), 2.87 (app t, J=6.3 Hz, 2H), 3.43 (dd, J=10.3, 5.0 Hz, 1H), 3.54 (m, 4H), 3.76 (dd, J=10.3, 6.5 Hz, 1H), 4.14 (s, 1H). The HRMS mass for C11H22N2O7SP [M−H]− was found to be 357.0880, which corresponds to the expected mass of 357.0885. The purity of the compound was determined to be >92%, using HPLC coupled with UV detection at 205 nm.

HPLC sample preparation protocol for total CoA and 4′-phosphopantetheine measurement: Samples were briefly washed with ice-cold PBS solution. Samples were sonicated thoroughly in 100 μl ice-cold PBS and centrifuged for 10-15 min at 4° C. to collect supernatant. Tris(2-carboxyethyl)phosphine hydrochloride (Sigma) (50 mM; 10 ul) was added to 50 μl sample supernatant and were incubated at room temperature for 15 min after vortex-mixing. Saturated ammonium sulfate solution or Millipore 3KD centrifugal filter units were used to remove proteins. The samples were centrifuged at 14,000 rpm for 15 min at 4° C. The clear supernatant (50 ul) or the filtrate was derivatized with 45 ul of ammonium 7-flurobenzo-2-oxa-1,3-doazole-4-sulfonate (SBD-F, Sigma) (1 mg/ml in borax buffer—0.1M containing 1 mM EDTA disodium, pH 9.5), and 5 ul ammonia solution (12.5% v/v, Merck Millipore) at 60° C. for 1 h. The derivatized samples were placed in a refrigerated autosampler (10° C.) in the Shimadzu HPLC system, and injected for total CoA and PPanSH analysis using optimized chromatographic separation conditions combined with fluorescence detection (described below).

Chromatography separation condition: Chromatographic analysis was performed with a Shimadzu LC-10AC liquid chromatograph, SCL-10A system controller, SIL-10AC automatic sample injector and LC-10AT solvent delivery system. Shimadzu RF-20Axs fluorescence detector was used for derivatized sample extract analysis. The fluorescence detector was set at excitation and emission wavelengths of 385 nm and 515 nm, respectively. Signal output was collected digitally with Shimadzu Labsolution software and post run analysis was performed. Chromatographic separation of the analytes was achieved with a Phenomenex Gemini C18 guard column (4×3 mm) connected to a Phenomenex Gemini NX-C18 analytical column (4.6×150 mm; 3 um particles) at 45° C. The two mobile phases consisted of A: 100 mM ammonium acetate buffer (pH 4.5) and B: acetonitrile. Flow rate was maintained at 0.8 ml/min with a slow gradient elution: 0% B till 7 min, 20% B at 20 min, 20% B at 22 min, 50% B at 23 min, maintained at 50% B till 27 min, 0% B at 28 min and 7-10 min for column re-equilibration.

Sample preparation for mass spectrometry and instrumental parameters: Samples were briefly washed with ice-cold PBS solution. Samples were then sonicated thoroughly in 100 μl ice-cold milliQ (MQ) water containing 50 mM Tris(2-carboxyethyl)phosphine hydrochloride. Subsequently 100 ul saturated ammonium sulfate was added to each sample and centrifuged for 20 min at 10° C., 16100 rcf to collect supernatant. To 150 μl of supernatant, 15 ul of ammonium hydroxide (12.5%) was added and 20 μl was injected for LC-MS (liquid chromatography-mass spectrometry) analysis. For mouse plasma analysis, 50 ul of MQ water containing 50 mM Tris (2-carboxyethyl)phosphine hydrochloride was added to 50 ul of plasma and processed further as mentioned above. Appropriate dilution series of standard CoA, PPanSH and PPanSH(D4) was processed similarly before analysis. The LC separation of metabolites were obtained using Phenomenex Gemini NX-C18 analytical column (4.6×150 mm; 3 um particles) at 45° C. The flow was maintained at 1 ml/min with optimized mobile phase gradient of MQ water (A), 200 mM NH4Ac in 95/5 MQ water/acetonitrile adjusted to pH 4.5 with acetic acid (B), and acetonitrile (C). The separated analytes were detected with positive mode mass spectrometry under unit resolution. The targeted Q1/Q3 mass/charge ions of PPanSH, PPanSH(D4), CoA and CoA(D4) were 359.1/261.1, 363.1/265.1, 768/261.1, and 772/265.1 respectively. The absolute concentration was finally calculated using linear regression analysis of respective standard compounds, except CoA(D4) which was estimated indirectly using CoA standards.

### Biological Assays

Drosophila S2 Cell Culture, RNA Interference, and CoA and 4′-phosphopantetheine treatment: Drosophila Schneider's S2 cells were maintained at 25° C. in Schneider's Drosophila medium (Invitrogen) supplemented with 10% heat inactivated fetal calf serum (Gibco) and antibiotics (penicillin/streptomycin, Invitrogen) under laboratory conditions. Synthesis of RNAi constructs and RNA interference (dsRNA) treatment was carried out as described previously (Siudeja K et al, EMBO Mol Med 3, 755-766 (2011)). Non-relevant (human gene; hMAZ) dsRNA was used as control. The cells were incubated for 4 days to induce an efficient knock-down. Cells were then subcultured, with or without CoA (Sigma-Aldrich, Cat. No: C4780—which is used for all the experiments wherever stated below) or 4′-phosphopantetheine (PPanSH) at different concentrations and were maintained for additional 3 days until analysis for rescue efficiency of the compounds was performed. The stock solutions of compounds were made in sterile water and stored in −20° C. until use.

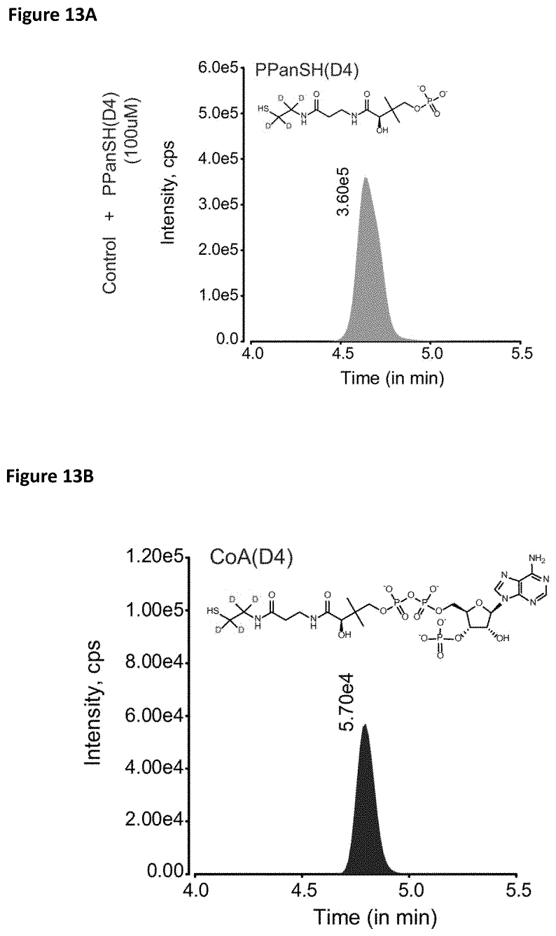

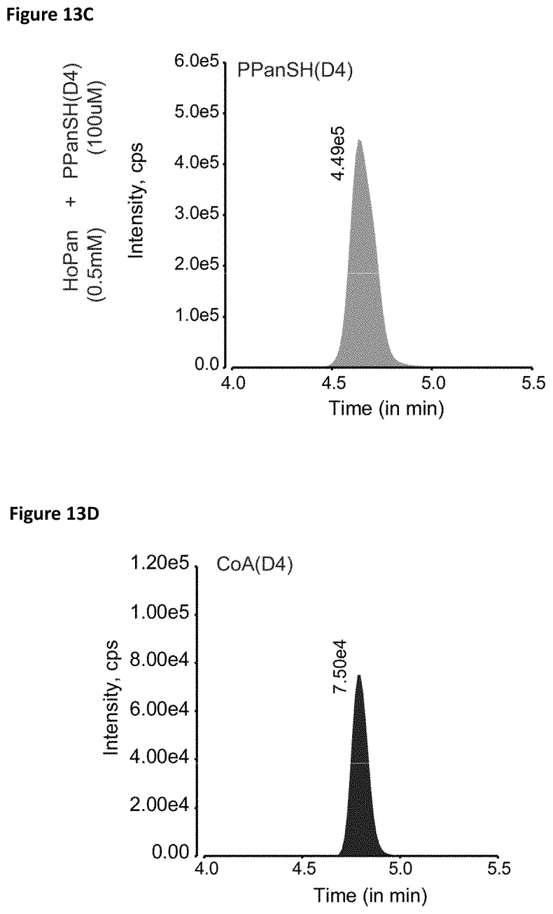

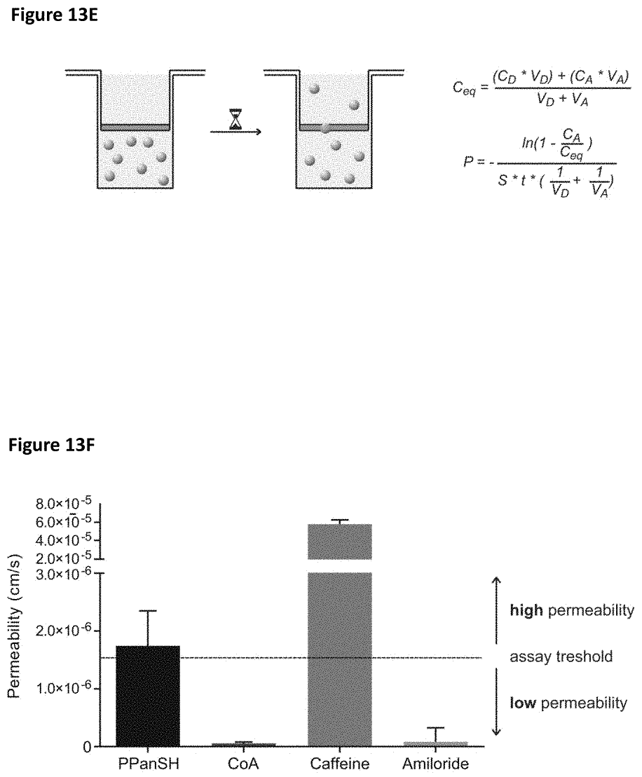

HoPan treatment of Drosophila S2 Cell in combination with CoA or 4′-phosphopantetheine treatment: Drosophila Schneider's S2 cells were maintained at standard conditions as explained above. Cells in the exponential phase of growth were used for all the experiments. Different indicated concentrations of CoA or 4′-phosphopantetheine (deuterium labelled PPanSH(D4) or unlabeled PPanSH) were added to S2 cells either in the presence or absence of 0.5 mM HoPan (Zhou Fang Pharm Chemical, China) for 48 h. Similarly, Drosophila S2 cells were treated with different concentrations of PPanSH(D4) at either 25° C. or 4° C. and cells were then harvested at various time points to access transport of PPanSH(D4). Stable isotope labelled PPanSH containing 4 deuterium atoms was purchased from Syncom (Groningen, The Netherlands) as a sodium salt (chemical structure is provided in FIG. 13A). As a read out cell count, intracellular total CoA and PPanSH levels (both labelled and unlabeled levels wherever appropriate) and histone acetylation levels were analyzed as explained below.

Drosophila S2 Cell Immunofluorescence Staining: For immunofluorescence Drosophila S2 cells were seeded on Poly-L-Lysine coated (Sigma-Aldrich) glass microscope slides and allowed to settle for 45 min. Cells were fixed with 3.7% formaldehyde (Sigma Aldrich) for 20 min, washed briefly with PBS+0.1% Triton-X-100 (Sigma Aldrich) and permeabilized with PBS+0.2% Triton-X-100 for 20 min. The slides were incubated in primary antibody (rabbit anti-AcLys, Cell Signaling Cat No: 9441, 1:500) to visualize histone acetylation levels in PBS+0.1% Triton-X-100 overnight and after an additional washing step in PBS+0.1% Triton-X-100 they were incubated in secondary goat anti-rabbit-Alexa488 antibody (Molecular Probes) for two hours at room temperature (RT). F-actin was detected with Rhodamin-Phalloidin (20 U/ml)(Invitrogen) and DNA by staining with DAPI (0.2 ug/ml) (Thermo Scientific). Finally the samples were mounted in 80% glycerol and analyzed using a Leica fluorescence microscope with Leica software. Adobe Photoshop and Illustrator (Adobe Systems Incorporated, San Jose, Calif., USA) were used for image assembly.

HoPan treatment of mammalian HEK293 Cells in combination with CoA and 4′-phosphopantetheine treatment: HEK293 cells were maintained in dMEM (Invitrogen) supplemented with 10% fetal calf serum (Gibco) and antibiotics (penicillin/streptomycin, Invitrogen). For HoPan treatment, cells were cultured in custom made dMEM without vitamin B5 (Thermo Scientific) supplemented with dialyzed FCS (Thermo Scientific). CoA or PPanSH was added to HEK293 cells for the final concentration of 25 uM, either in the presence or absence of HoPan (0.5 mM) for 4 days, followed by analysis for phenotype and rescue efficiency of CoA and PPanSH.

Knock-down of COASY by siRNA in mammalian HEK293 cells: HEK293 were maintained as described above. HEK293 were transfected with 200 nM COASY siRNA (GE Healthcare human COASY 80347 smartpool Cat no: M-006751-00-0010) or non-targeting siRNA (GE Healthcare Cat no: D-001206-13-20) using lipofectamine 2000 (Invitrogen) 4 h after transfection CoA was added in a final concentration of 25 uM. Cells were cultured for 3 days and then harvested for HPLC analysis of total CoA and PPanSH levels and Western blot (histone acetylation) as described below.

Western blot analysis and Antibodies: For Western blot analysis, cells were collected and washed with phosphate buffer saline (PBS), followed by centrifugation. The cells were lysed and sonicated in 1X Laemmli Sample Buffer and boiled for 5 min with 5% β-mercaptoethanol (Sigma). Protein content was determined using DC protein assay (BioRad). Equal amounts of protein were loaded on a 10 or 12.5% SDS-PAGE gel, transferred onto PVDF membranes and blocked with 5% milk in PBS/0.1% Tween, followed by overnight incubation with primary antibodies. The primary antibodies used were: rabbit-anti dPANK/fbl, 1:4000 Eurogentec custom made as described previously5, mouse anti-tubulin (Sigma Aldrich Cat no: T5168, 1:5000), anti-acetyl-Histone3 (Active Motif Cat no: 39139, 1:2000), anti GAPDH (Fitzgerald Cat no: 10R-G109a, 1:10000), rabbit anti COASY (Abcam Cat no: AB129012, 1:1000). Appropriate HRP-conjugated secondary antibodies (Amersham) were used and detection was performed using enhanced chemi-luminescence (Pierce cat nog: 32106) and Amersham hyperfilm (GE Healthcare). Band intensities were quantified with Image-studio software.

C. elegans Media and Strains: Standard culturing conditions were used for C. elegans maintenance at 20° C. N2 strain was used as a wild-type control. VC927, the PANK deletion mutant pnk-1 (ok1435)I/hT2[bli-4(e937) let-? (q782)qIs48](I; III), was obtained from the Caenorhabditis Genetics Center. To obtain synchronous cultures, worms were bleached with hypochlorite, and allowed to hatch in M9 buffer (3 g KH₂PO₄, 6 g Na₂HPO₄, 5 g NaCl, 1 ml 1 M MgSO₄, H₂O to 1 liter) overnight and cultured on standard Nematode Growth Medium (NGM) plates seeded with OP50 strain of Escherichia coli.

C. elegans Motility Assay: After synchronization, L1 C. elegans were grown on control NMG plates or NGM plates containing various concentrations of CoA. One-day old adults were placed in a drop of M9 buffer and allowed to recover for 30 sec. During the following 30 sec, the number of body bends was counted. A movement was scored as a bend when both the anterior and posterior ends of the animal turned to the same side. At least 15 worms were scored per condition and each experiment was repeated thrice. The sequential light microscopy images demonstrating movements of C. elegans in M9 buffer were captured using Leica MZ16 FA microscope at 32× magnification within the time frame of 1 sec and processed using ImageJ (National Institutes of Health, Maryland, USA) and Adobe Photoshop (Adobe Systems Incorporated, San Jose, Calif., USA).

Drosophila Maintenance and Crosses: Drosophila melanogaster stocks/crosses were raised on standard cornmeal agar fly food (containing water, agar 17 g/L, sugar 54 g/L, yeast extract 26 g/L and nipagin 1.3 g/L) at 25° C. The stocks were either obtained from the Bloomington Stock Centre (Indiana University, USA), VDRC (Vienna Drosophila RNAi Collection, Vienna, Austria) or from the Exelixis Collection (Harvard Medical School) and rebalanced to generate eGFP-positive balancers. The stocks used were: w1118; dPANK/fbl1 hypomorph5,6; dPANK/fblnull (y[1] w[*]; Mi{y[+mDint2]=MIC}fbl[MI04001]/TM3, Sb[1] Ser[1], Bloomington 36941); dPPCDC mutant (w[1118], PBac{w[+mC]=WH}Ppcdc[f00839]/CyO, Bloomington 18377); UAS-dPPCDCRNAi line (VDRC 104495); dCOASY mutant (PBac{RB}Ppat-Dpck[e00492], Exelixis). The UAS-RNAi constructs were expressed ubiquitously using the Actin-Gal4 drivers (y[1] w[*]; P{w[+mC]=Act5C-GAL4}25FO1/CyO, y[+], Bloomington 4414). Heterozygous flies/larvae for the mutants and the Actin-Gal4 driver crossed to isogenic w1118 flies (Actin-Gal4/+) were used as controls for the RNAi-constructs expressing flies.

Drosophila Larval Collection and Larval Count Experiment: One week old flies (in the ratio 10 females and 5 males) were kept on 5 ml of standard fly food in a vial at 25° C. with or without various concentrations of CoA or Vitamin B5 (Sigma). The flies were allowed to lay eggs for 2 days and parent flies were then discarded. The L1, L2 and L3 larvae were collected from the food with 20% sucrose at appropriate time (day 4, 6 and 8 respectively) for larval counting and stored in −80° C. until analysis. The pupal count was performed between 10-12 days.

Drosophila HoPan Toxicity and CoA Rescue Experiment: One week old w1118 flies (in the ratio 10 females and 5 males) were kept in vials containing standard fly food with or without HoPan and CoA at indicated concentrations. The flies were allowed to lay eggs for 2 days, after which the adults were discarded. The resulting offspring were allowed to develop. The numbers of flies which eclosed were counted to evaluate HoPan toxicity and CoA rescue efficiency.

Drosophila Life Span: One-day old adults of Drosophila homozygous mutants or RNAi-constructs expressing lines, were collected with appropriate controls and were kept on standard fly food at 25° C. with or without CoA or Vitamin B5 (Sigma) at necessary concentration (50 ul added on top of the fly food and dried). The flies were counted every 12-24 hrs and flipped to new fly food vials with or without CoA or Vitamin B5.

Drosophila Ovary Rescue Experiment: UAS-dPPCDC RNAi constructs were ubiquitously expressed under the control of Actin-Gal4. The crosses were raised at 25° C. F1 RNAi-construct expressing females and control females were collected shortly after eclosion and transferred to standard fly food or food containing Vitamin B5 or CoA (18 mM). Flies were maintained for 2 days on this food at 25° C. After this period extra yeast and w1118 control males were added and the crosses were kept at 25° C. for another 2 days. After this 4 day period ovaries were dissected and stained for further analysis. The vials (or plates) from the crosses (with eggs that were being laid during the 4 day period of CoA rescue) were kept for another 10 days and offspring numbers were counted after eclosion.

RNA Isolation, Quantitative Real-Time PCR, and Primers: Drosophila larvae and samples of 1-day old adult flies were collected for homozygous dPPCDC mutants, dPPCDC RNAi-construct expressing lines and for homozygous dCOASY mutants, followed by brief washing with PBS. The samples were lysed in TRIZOL (Invitrogen) for RNA extraction and reverse transcribed using M-MLV (Invitrogen) and oligo(dt) 12-18 (Invitrogen). SYBR green (Bio-Rad) and Bio-Rad Real-Time PCR with specific primers were used for gene expression level analysis. The expression levels were normalized for rp49 (house-keeping gene). The Primer sequences used were dPPCDC (TGCACCTGCGATGAATACCC; TCGGCTGAAAGGCGGATAAC (SEQ ID NO: 1)), dCOASY (GGCTGTGCGGCGGATTATTG (SEQ ID NO: 2); CGGGTTAAAGGCTGCTCTGG (SEQ ID NO: 3)) and rp49 (GCACCAAGCACTTCATCC (SEQ ID NO: 4); CGATCTCGCCGCAGTAAA (SEQ ID NO: 5)) (Biolegio).

Drosophila Ovary dissection and staining: Drosophila ovaries were collected in cold PBS and fixed in 4% formaldehyde (from methanol-free 16% Formaldehyde Solution, Thermo Scientific) for 45 min at RT. The fixed tissue was washed in PBS+0.1% Triton-X-100 for 1 hour at RT and afterwards permeabilized in PBS+0.2% Triton-X-100 for 1 hour. Finally the ovaries were stained with Rhodamin-Phalloidin (20 U/ml) to detect F-actin and DAPI (0.2 μg/ml) for DNA. Finally the samples were mounted in 80% glycerol and analyzed on a Zeiss-LSM780 NLO confocal microscope with Zeiss Zen software. Adobe Photoshop and Illustrator (Adobe Systems Incorporated, San Jose, Calif., USA) were used for image assembly.

PAMPA assay procedure: Parallel Artificial Membrane Permeability Assay (PAMPA) was performed and processed according to manufacturer's instructions (BD Gentest Pre-coated PAMPA plates). Briefly, two superimposed wells are separated by an artificial lipid-oil-lipid membrane. The compound of interest (PPanSH, CoA, caffeine, amiloride) was added to the bottom well in phosphate-buffered saline, whereas the top well was filled with phosphate-buffered saline alone. After 5 h of incubation at room temperature, concentrations of the different compounds were measured using UV-VIS absorption spectroscopy (BMG Labtech SPECTROstar Omega) along with calibration curves for all compounds. The permeability efficiency was further calculated according to manufacturer's instructions. For caffeine and amiloride, four replicates were performed; for PPanSH and CoA twelve replicates were performed. Caffeine and amiloride were obtained from Sigma.

Mice and CoA intravenous injection study: Adult male mice of C57BL/6J 129/SvJ mixed genetic background were used for this study. Two mice, (approximately 25-30 g wt) were used for each condition. 0.1 mg or 0.5 mg CoA in 0.25 ml saline solution was injected intravenously (i.v) into the tail vein. Saline solution (0.25 ml) was injected to control groups. After 30 min and 6 h blood samples were collected and further processed to obtain plasma followed by sample preparation for HPLC or LC-MS analysis as indicated below. All animal studies were approved by the Ethics Committee of the Foundation IRCCS Neurological Institute C. Besta, in accordance with guidelines of the Italian Ministry of Health: Project no. BT4/2014. The use and care of animals followed the Italian Law D.L. 116/1992 and the EU directive 2010/63/EU.

Statistical Analysis: All experimental results are presented as mean of at least 3 independent experiments±SD, unless otherwise stated. Statistical significance was determined by a two-tailed unpaired Student's t test between appropriate groups wherever applicable. For life span survival curve, more than 80 flies were used in each group and statistical significance was determined using Log-rank (Mantel-Cox) test. Statistical p values≤0.05 were considered significant (*p≤0.05, **p≤0.01, ***p≤0.001). Data were analyzed using GraphPad Prism (GraphPad Software, San Diego, Calif., USA).

### Example 1. CoA Supplementation Rescues Phenotypes Induced by Impaired CoA de Novo Biosynthesis

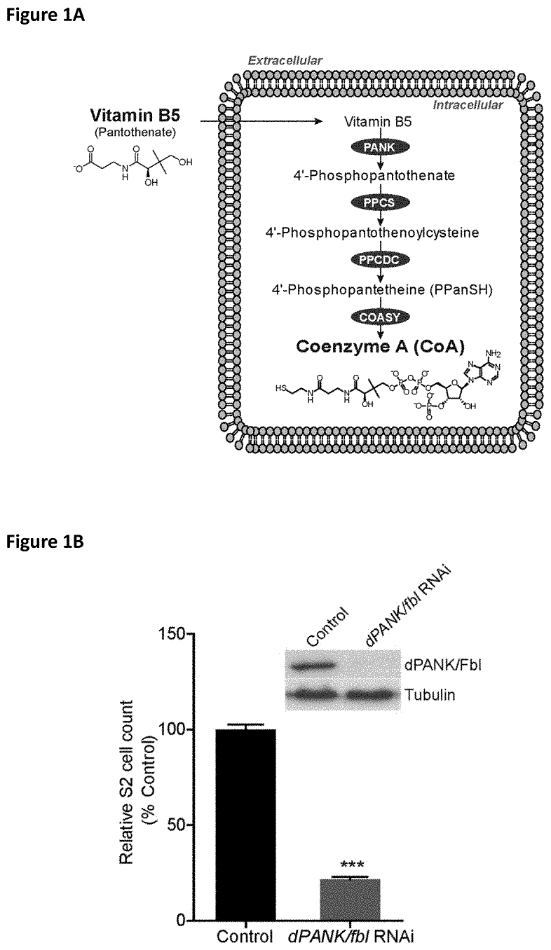

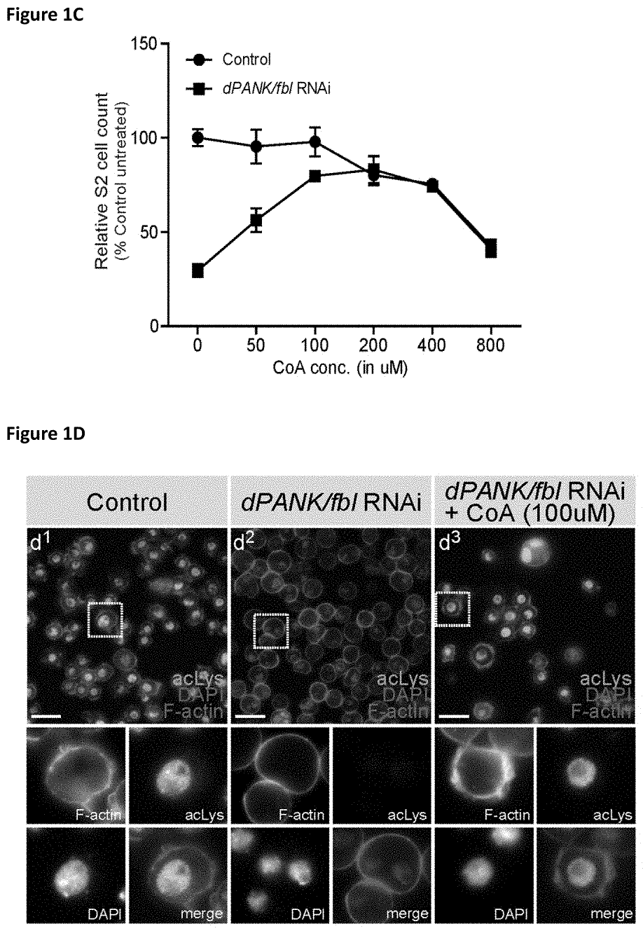

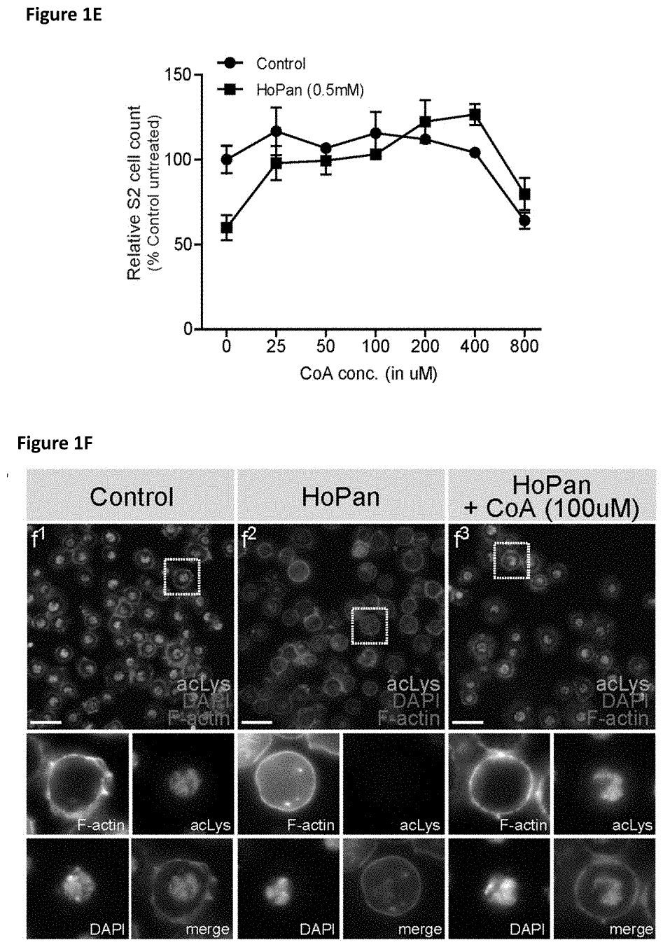

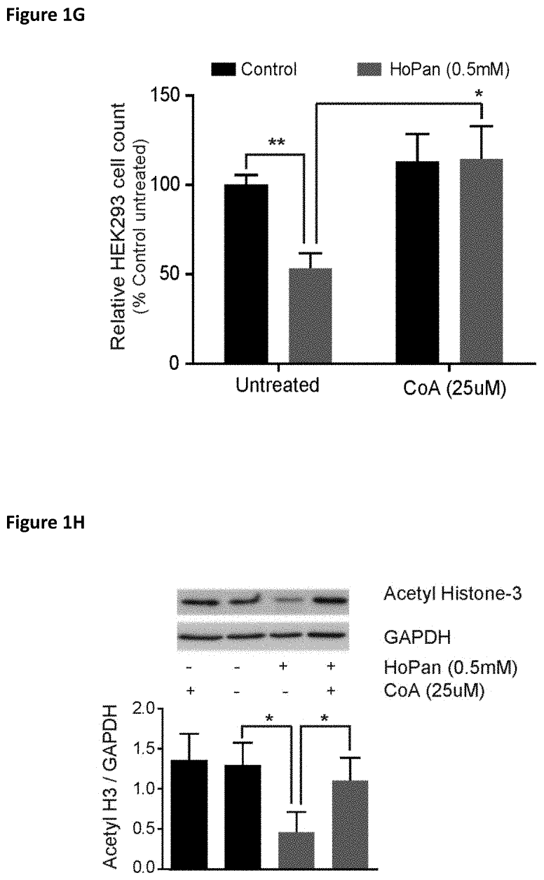

In order to answer the question of whether cells are able to obtain CoA from sources other than classic de novo biosynthesis (FIG. 1A), it was first determined whether extracellular sources of CoA could serve as a supply for intracellular CoA in eukaryotic cells. RNA interference was used to induce PANK (first enzymatic step) depletion to block the de novo biosynthesis route and to create a CoA-depleted phenotype. Subsequently the rescue potential of exogenous CoA was tested. PANK depletion by RNA interference in Drosophila cultured S2 cells (FIG. 1B inset) was associated with a reduction in cell count (FIG. 1B) and histone acetylation levels (FIG. 1D), as previously demonstrated in Siudeja K et al, 2011 supra.

Addition of CoA to the medium of the cultured cells rescued the cell count in a concentration-dependent manner (FIG. 1C) and histone acetylation phenotypes (FIG. 1D). Next, it was determined whether this rescue applies to other cell types and systems of impaired CoA biosynthesis. Treating Drosophila S2 cells with the chemical PANK inhibitor Hopantenate (HoPan) (Zhang Y M et al, Chem Biol 14, 291-302 (2007)), also induced a decrease in cell count (FIG. 1E) and histone acetylation levels (FIG. 1F). This HoPan-induced phenotype was also rescued by direct supplementation of CoA to the medium of the cells (Fig). Next, the effects of HoPan in mammalian HEK293 cells were assessed to address the possibility that the beneficial effects of exogenous CoA are insect cell-specific. When HEK293 cells were treated with HoPan, they showed a phenotype similar to Drosophila S2 cells, with decreased cell count and impaired histone acetylation. When CoA was added to the culture medium both the decreased cell count (FIG. 1G) and the impaired histone acetylation phenotypes (FIG. 1H) were rescued. These in vitro results confirmed the potency of exogenous CoA to rescue phenotypes induced by impaired PANK in diverse cellular systems.

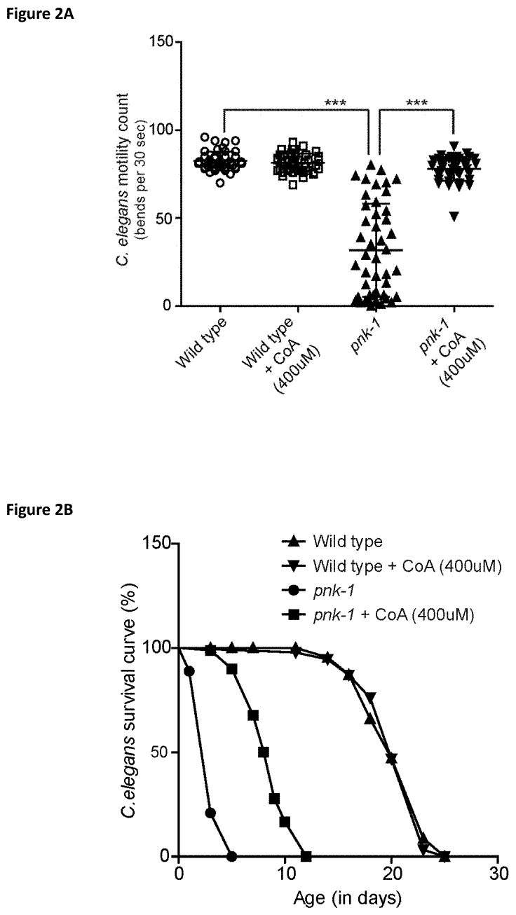

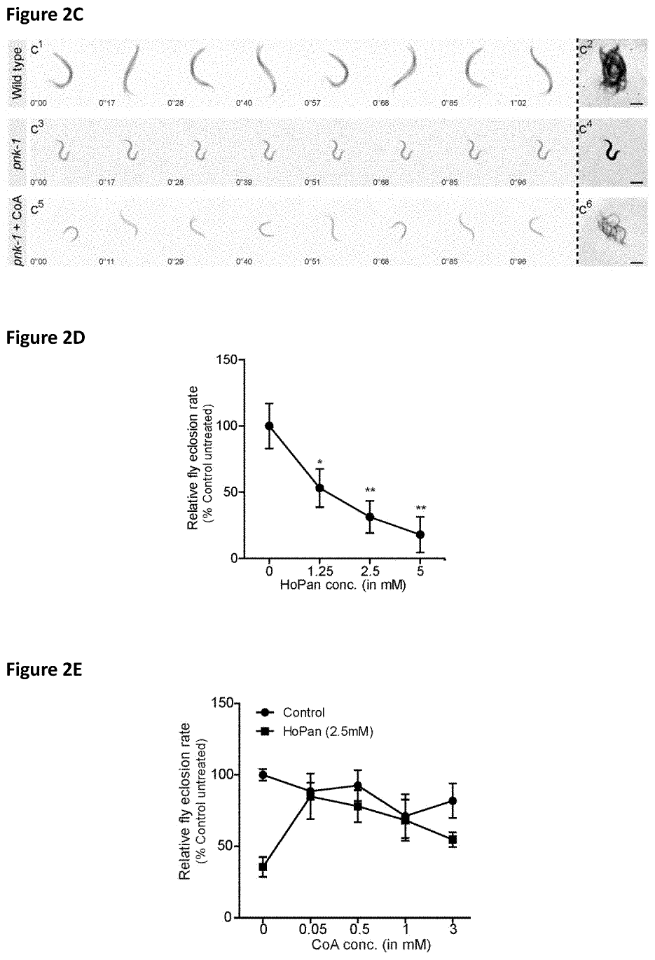

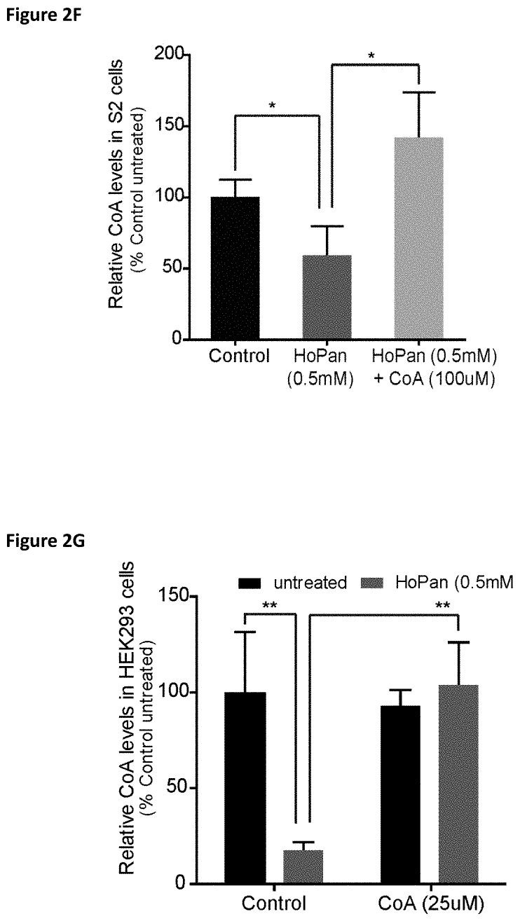

Homozygous Caenorhabditis elegans (C. elegans) pantothenate kinase (pnk-1) mutants were used to test the effect of CoA supplementation in vivo. These mutants showed decreased motility (FIG. 2A, FIG. 2C) and a decreased lifespan (FIG. 2B). Addition of CoA to the food of these mutants improved these phenotypes significantly (FIGS. 2A-2C and FIG. S1). Furthermore, when a Drosophila w¹¹¹⁸ control fly line was treated with HoPan, larval lethality was induced and a decreased eclosion (emerging from the pupal case) rate was observed (FIG. 2D). This HoPan-induced phenotype was fully rescued by the addition of CoA to the food of the larvae (FIG. 2E).

These data demonstrate that supplementation of CoA reverts the phenotypes arising from impaired de novo CoA biosynthesis, an effect that is conserved across diverse eukaryotic cell types and organisms.

### Example 2. External Supplementation of CoA Influences Intracellular Levels of CoA

The observed rescue effect in Example 1 could occur in several ways. Either intracellular CoA levels are restored, or rescue is independent of the restoration of CoA levels in the cell. If the latter is true, intracellular levels of CoA would not be restored by exogenous CoA. To investigate this, a sensitive HPLC method was developed that included pre-column thiol-specific derivatization of samples with ammonium 7-fluorobenzofurazan-4-sulfonate (SBDF), followed by chromatographic separation by gradient elution on a C18 column and fluorescence detection. The HPLC CoA analysis showed that intracellular CoA levels were significantly reduced in extracts of HoPan treated S2 and HEK293 cells. Addition of CoA to the culture medium restored the intracellular concentration of CoA (FIGS. 2F and 2G). These results suggest that extracellular CoA exerted its effects in CoA-depleted cells by increasing and thereby “normalizing” intracellular CoA concentrations. This influence appears to be independent of PANK activity. Therefore, exogenous CoA can increase intracellular CoA levels, bypassing the canonical de novo CoA biosynthetic pathway. The mechanism behind this alternative CoA route, however, is not previously known.

### Example 3. Degradation of CoA to 4′-Phosphopantetheine, a Serum-Stable Metabolite, in Serum

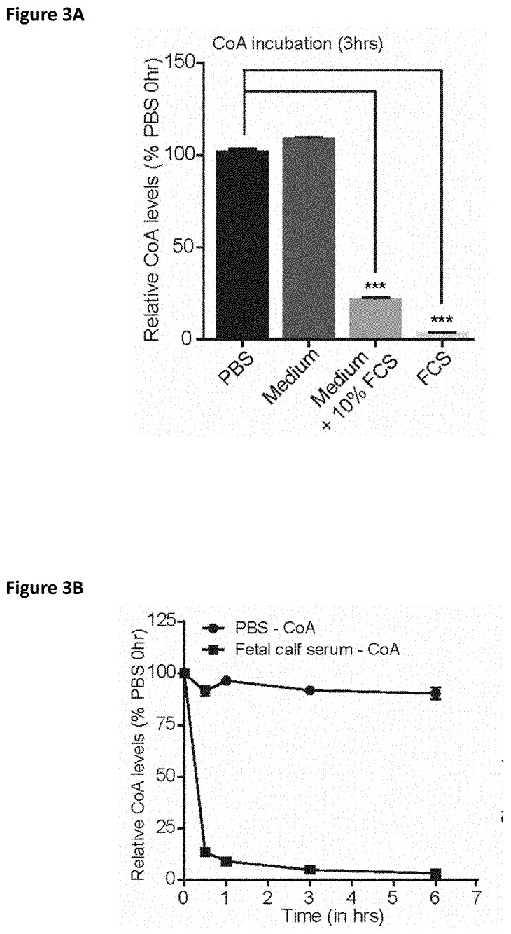

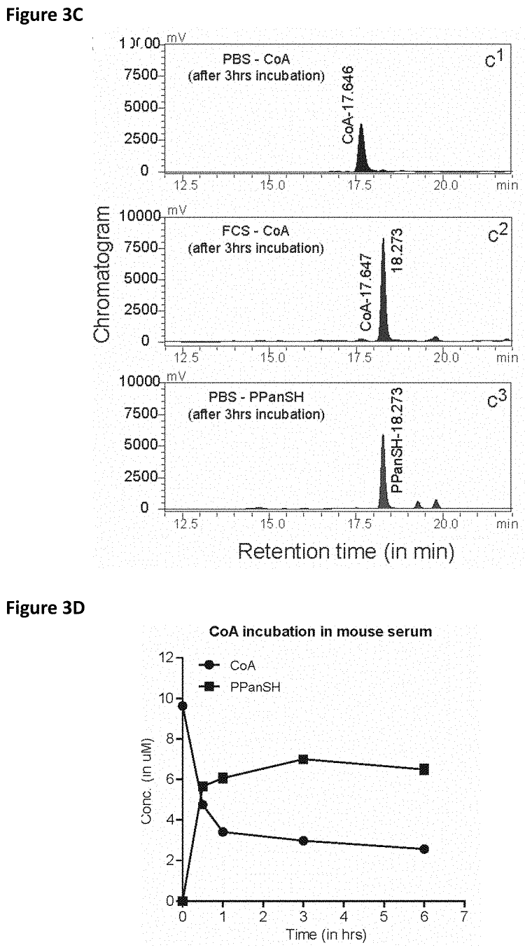

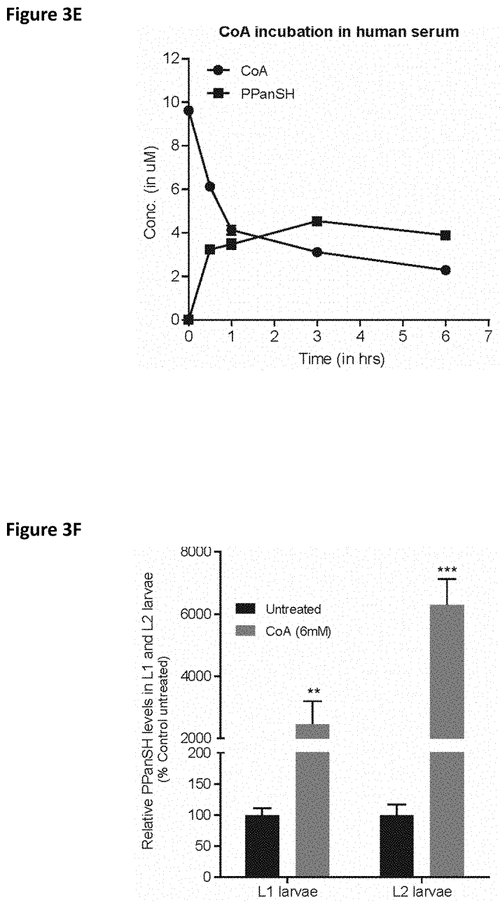

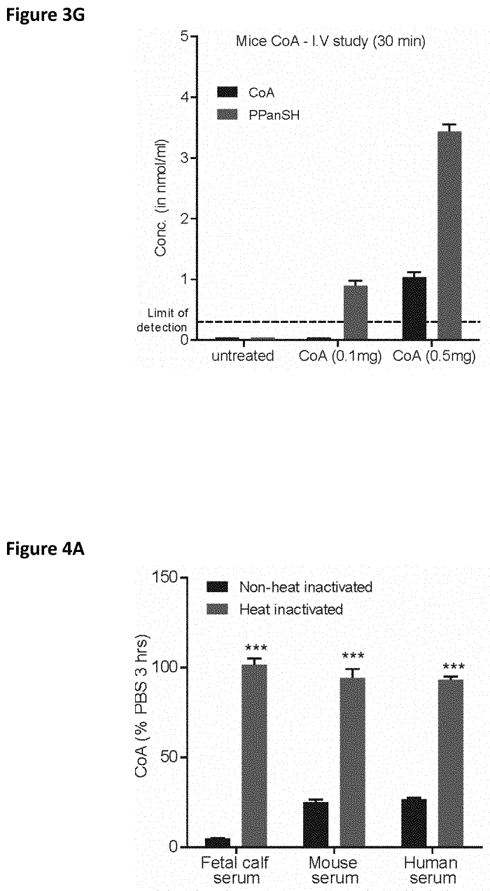

The observations in Example 2 indicate that either 1) CoA can enter cells directly, although such a transport process has not been described; or 2) CoA is converted to an intermediate product that enters the cell and is converted back to CoA in a PANK-independent manner. Previous research found that CoA is not stable in liver extracts and degrades to 50% at −20° C. after a week (Shibata et al., Anal Biochem 430:151-155 (2012)); however, the stability of CoA in an extracellular environment such as in aqueous or in standard cell culture medium is unknown. Moreover, these early reports did not identify specific degraded or converted products. The stability of CoA in PBS, serum-free medium, medium containing fetal calf (FCS) serum and in fetal calf serum was measured. HPLC analysis revealed that CoA was relatively stable in PBS and serum free medium, with >95% of the initial concentration still present after 3 hours. However, in the presence of fetal calf serum, CoA was rapidly degraded with only 10% of the initial concentration was detectable after three hours (FIG. 3A). Detailed stability analysis at different time points in PBS and fetal calf serum revealed that 90% of CoA was already degraded after 30 minutes in fetal calf serum (FIG. 3B). Disappearance of CoA coincided with the appearance of one unknown thiol-containing product in the HPLC chromatogram that migrated at 18.273 minutes and remained stable over the whole time course (FIG. 3C). It was hypothesized that the peak could be a CoA degradation product such as dephospho-CoA, 4′-phosphopantetheine (PpanSH), or pantetheine (Leonardi et al., 2005 supra; Strauss, Comp. Nat. Prod. 2:351-410 (2010)).

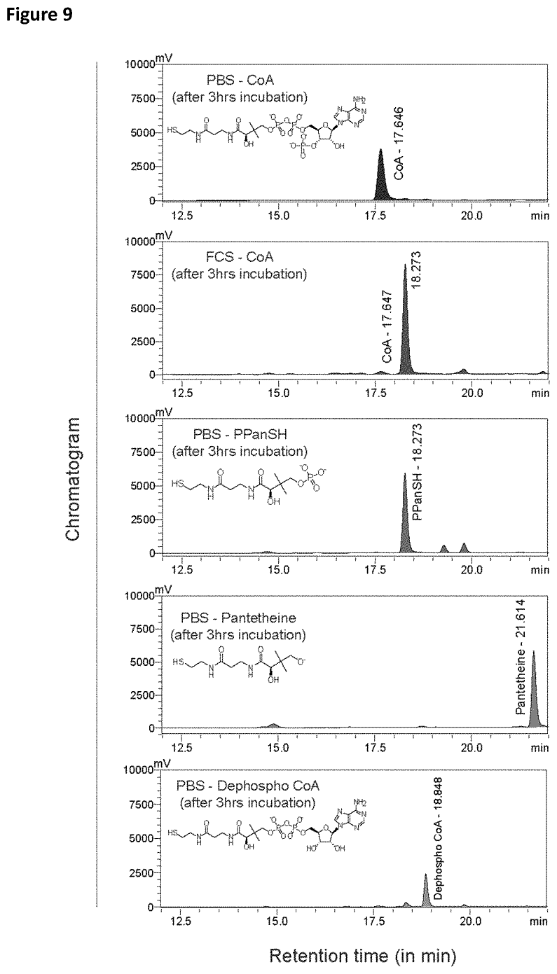

4′-Phosphopantetheine was chemically synthesized as shown in FIG. 8. Further HPLC analysis and comparison with standards demonstrated that the thiol-containing degradation product of CoA was neither dephospho-CoA nor pantetheine (FIG. 9), but it exactly matched the retention time of 4′-phosphopantetheine standard (FIG. 3C). These results indicate that CoA is converted into 4′-phosphopantetheine in serum and is stable. The conversion of CoA to 4′-phosphopantetheine was further investigated in mouse serum and in human serum. In both sera, CoA was also converted to 4′-phosphopantetheine (FIGS. 3D and 3E).

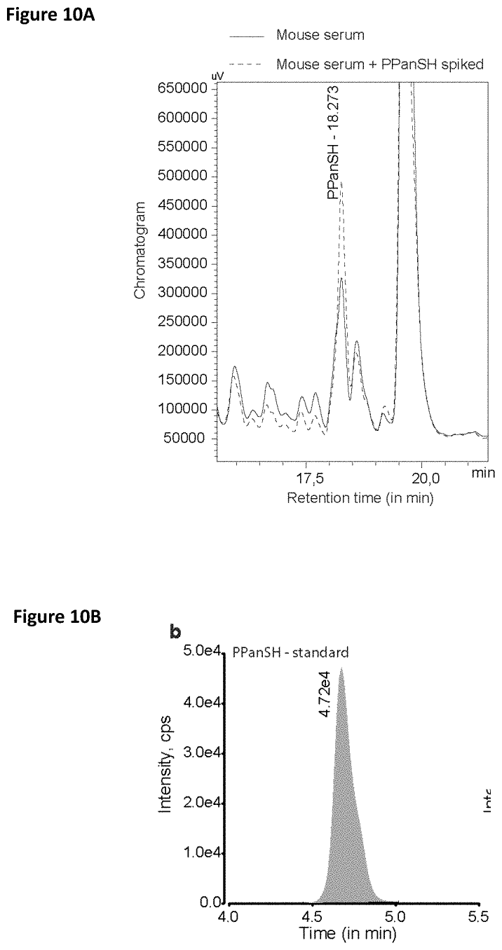

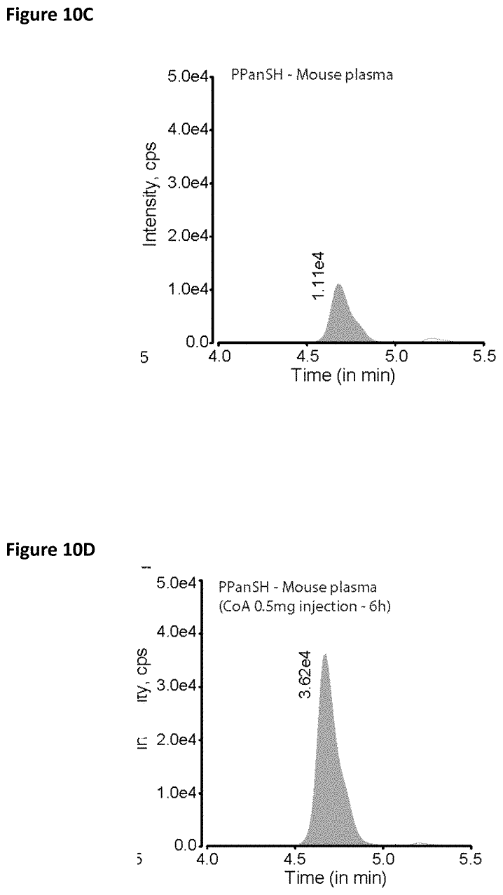

To investigate whether this conversion also occurs in vivo, Drosophila larvae were fed CoA, and L1 and L2 stage larval extracts were obtained after 2 days and 3 days of feeding, respectively. HPLC analysis showed that externally added CoA resulted in increased levels of 4′-phosphopantetheine in both L1 (>20 fold) and L2 larvae (>60 fold) (FIG. 3F). To investigate whether this conversion also occurs in higher organisms, different concentrations of CoA were injected intravenously into adult mice, and plasma was collected after 30 min and 6 hrs. HPLC analysis showed that the injected CoA was rapidly converted to 4′-phosphopantetheine after 30 minutes (FIG. 3G). Mass spectrometry demonstrated that 4′-phosphopantetheine is still present in the plasma 6 hrs after CoA injection. (FIG. 10D).

These data indicate that CoA is converted into 4′-phosphopantetheine in vitro and in vivo. Furthermore these results suggest that 4′-phosphopantetheine could be the principal molecule that is taken up by CoA-depleted cells, converted back into CoA intracellularly, which in turn results in rescue of the CoA-depleted phenotype.

### Example 4. Conversion of CoA into 4′-Phosphopantetheine in Serum Depends on Ecto-Nucleotide Pyrophosphatases

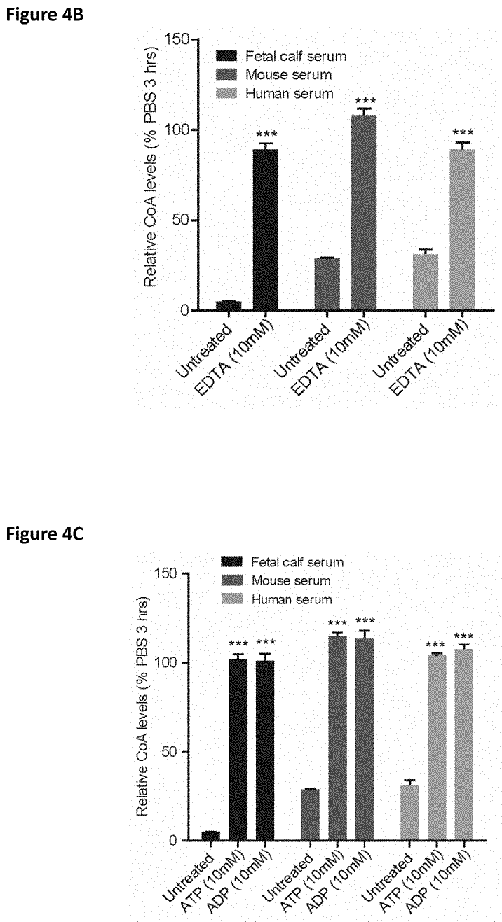

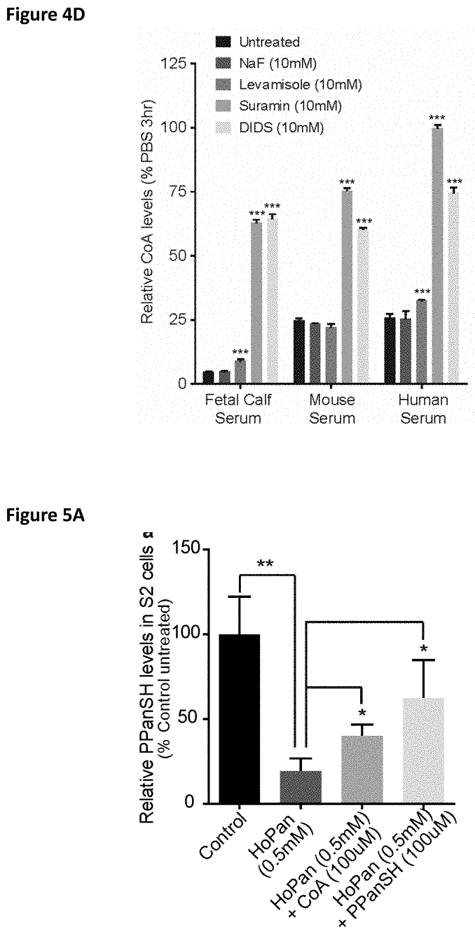

The factors that convert CoA into 4′-phosphopantetheine in serum were identified. Serum from various species (fetal calf, mouse and human) was pre-conditioned, and CoA conversion into 4′-phosphopantetheine was assessed. First, the effect of heat inactivation of the serum was studied. HPLC analysis showed that heating the serum at 56° C. for 30 min completely abolished the conversion of CoA to 4′-phosphopantetheine (FIG. 4A), indicating the involvement of enzymes or proteins in this process. Second, the conversion of CoA to 4′-phosphopantetheine requires the hydrolysis of a phosphoanhydride bond, which is typically catalyzed by (pyro)phosphatases or hydrolases. The majority of enzymes in the known family of (pyro)phosphatases and hydrolases depend on metal ions for their activity. To test these candidates, EDTA was added to serum to chelate metal ions.

Treatment of serum with EDTA completely prevented the formation of 4′-phosphopantetheine (FIG. 4B). This strongly suggests that metal ions are required for the CoA conversion. The most likely hydrolase or (pyro)phosphatase candidates, which possess the ability to convert CoA and which are metal-ion dependent for their activity, are nudix hydrolases, alkaline phosphatases and ectonucleotide pyrophosphatases (ENPPs) (AbdelRaheim et al., BMC Biochem. 3:5 (2002); Franklin et al., Biochim. Biophys. Acta. 230:105-116 (1971); Kang et al., J. Bacteriol. 185:4110-4118 (2003); Novelli et al., J. Biol. Chem. 206:533-545 (1954); Reilly et al., J. Biochem. 144:655-663 (2008); Shibata et al., J. Nutrition 113:2107-2115 (1983); Skrede, Eur. J. Biochem. 38:401-407 (1973); and Trams et al., Biochem. Biophys. Acta. 163:472-482 (1968)). These candidate enzymes are also known for their ability to hydrolyze ATP and ADP (Fernandez et al., Am. Soc. Vet. Clin. Pathol. 36:223-233 (2007); McLennan, Cell Mol. Life Sci. 63:123-143 (2006); and Rucker et al., Mol. Cell Biochem. 306:247-254 (2007)).

As a result, the conversion of CoA into 4′-phosphopantetheine in serum after addition of excess ATP and ADP was tested. Both competitively blocked the conversion in all sera tested, further underscoring the involvement of one of these enzymes (FIG. 4C). Alkaline phosphatase and ENPPs have been shown to be excreted by cells and to be present in serum (Fernandez et al., 2007 supra; and Jansen et al., Structure 20:1948-1959 (2012)). Nudix hydrolases have been shown to be intracellular hydrolases of CoA (AbdelRaheim et al., 2002 supra; Reilly et al., 2008 supra; McLennan, 2006 supra); however, a possible extracellular role for this class of hydrolases cannot be excluded.

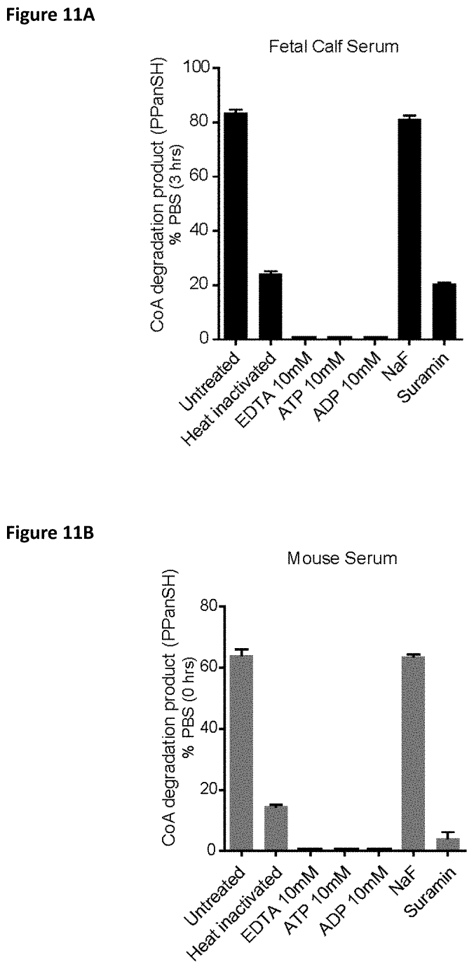

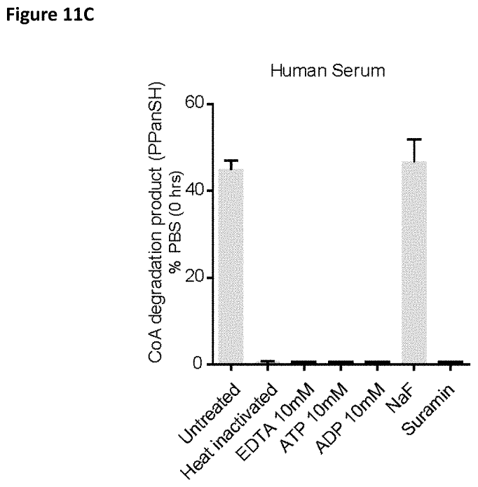

Sodium fluoride (NaF) selectively inhibits nudix hydrolases and levamisole selectively inhibits alkaline phosphatase while suramin and 4,4′-diisothiocyanatostilbene-2,2′ disulphonic acid (DIDS) selectively inhibit ENPPs (AbdelRaheim et al., 2002 supra; Rucker et al, 2007 supra; Furstenau et al., Platelets 17:84-91 (2006); Grobben et al., Br. J. Pharmacol. 130:139-145 (2000); and Gu et al., The Analyst 138:2427-2431 (2013). When used herein, only suramin and DIDS were able to inhibit the degradation of CoA into 4′-phosphopantetheine in all sera tested. Levamisole, and sodium fluoride (NaF) showed only mild or no inhibition of CoA degradation into 4′-phosphopantetheine (FIG. 4D). These experiments identify ENPPs as the most likely class of enzymes to hydrolyze CoA into 4′-phosphopantetheine in serum. This is supported by the observation that in all of the CoA serum stability experiments listed above; there is an inverse correlation between the levels of CoA and 4′-phosphopantetheine (FIGS. 11A-11C).

### Example 5. External Supplementation of 4′-Phosphopantetheine Rescues CoA-Depleted Phenotypes

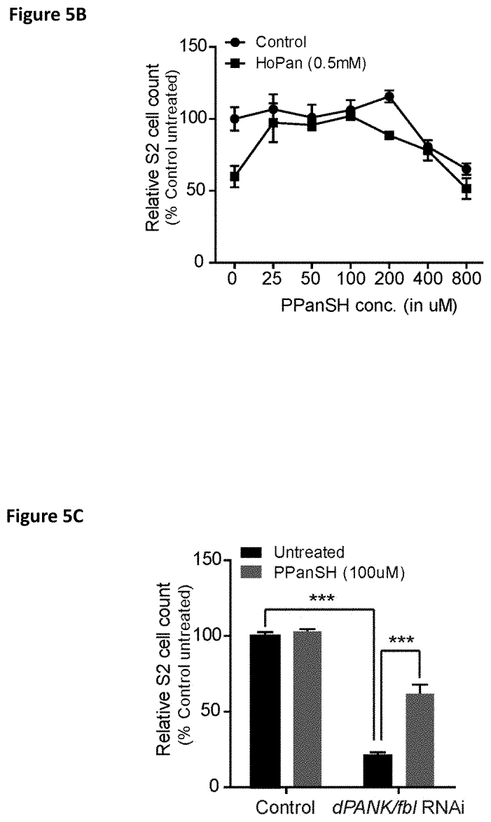

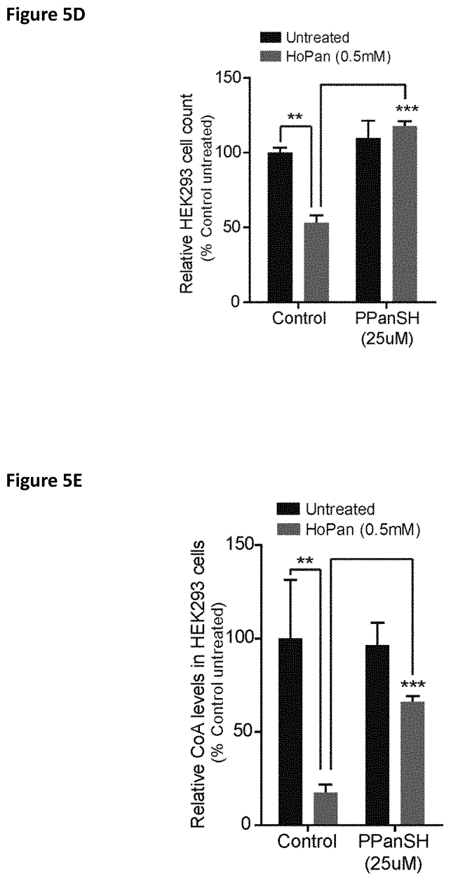

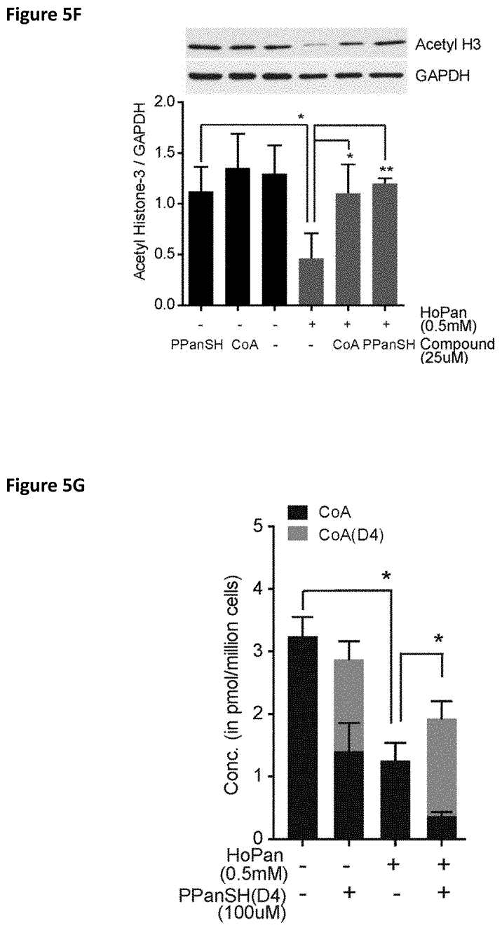

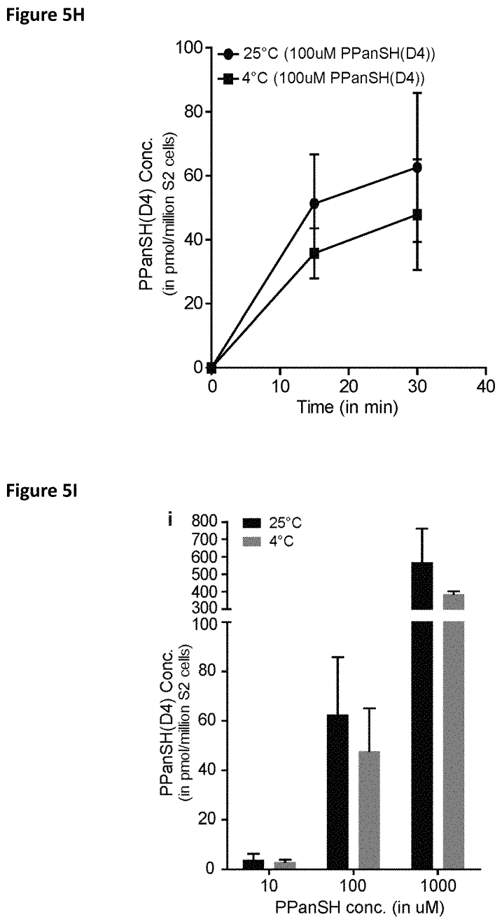

PANK impairment results not only in decreased CoA levels but also in decreased levels of 4′-phosphopantetheine. Therefore, addition of 4′-phosphopantetheine to CoA-depleted cells should rescue the induced phenotypes. HPLC analysis of HoPan treated Drosophila S2 cells indeed showed reduced levels of 4′-phosphopantetheine, and external supplementation with either CoA or 4′-phosphopantetheine significantly increased intracellular levels of 4′-phosphopantetheine (FIG. 5A). Moreover, when 4′-phosphopantetheine was added to Drosophila S2 cells treated with HoPan (FIG. 5B) or dPANK/fbl RNAi (FIG. 5C) the

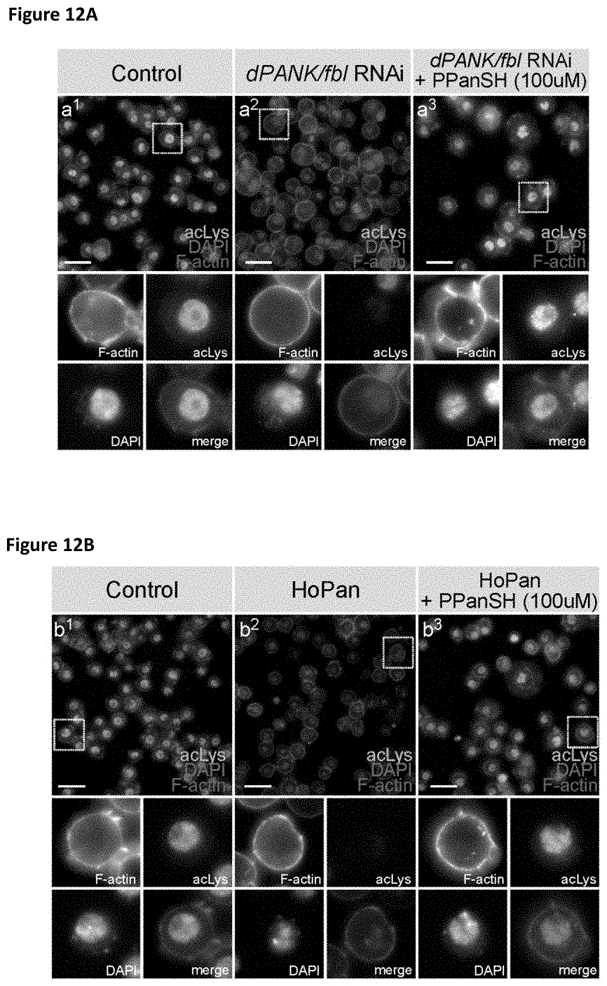

CoA-depleted phenotype was again rescued. 4′-Phosphopantetheine supplementation also rescued the histone acetylation defect in Drosophila S2 cells treated with dPANK/fbl RNAi (FIG. 12A) or HoPan (FIG. 12B). Finally, the rescue effect of 4′-phosphopantetheine in HoPan-treated mammalian HEK293 cells was tested. It also rescued the HoPan-induced reduction in cell count (FIG. 5D), intracellular CoA level (FIG. 5E) and histone acetylation (FIG. 5F).

Next, it was investigated whether intact 4′-phosphopantetheine enters cells and whether it was subsequently converted into CoA. First, intact Drosophila S2 cells in culture were treated with stable isotope-labelled 4′-phosphopantetheine under various conditions. Mass spectrometry analysis was used to measure the levels of stable isotope-labelled CoA within the harvested cell extracts. When labelled 4′-phosphopantetheine is added to the cell culture medium under standard culturing conditions, labelled CoA was detected in harvested cell extracts (FIG. 5G).

In the presence of HoPan, CoA levels were decreased and replenished in the form of labelled CoA when labelled 4′-phosphopantetheine was added. These data demonstrate that exogenously provided 4′-phosphopantetheine is able to enter cells and intracellularly converted into CoA under normal culturing conditions and under conditions of impaired CoA biosynthesis by HoPan (FIGS. 13A-13D).

Next, the mechanism of transport of 4′-phosphopantetheine across the cell membrane was assessed. Thirty minutes after the incubation of cells with labelled 4′-phosphopantetheine, intracellular labelled 4′-phosphopantetheine was detected in cells cultured at 25° C. (the normal culturing temperature of S2 cells) and at 4° C. There was no significant difference in the intracellular concentration of labelled 4′-phosphopantetheine between these two conditions (FIG. 5H). A concentration series (10-100-1000 μM) of labelled 4′-phosphopantetheine was added to cells treated as described above. The levels of intracellular 4′-phosphopantetheine increased to the same extend as externally added increased concentrations of 4′-phosphopantetheine (FIG. 5I). These results indicate that the capacity of cells to accumulate the externally provided 4′-phosphopantetheine is not influenced by temperature and is determined by extracellularly provided concentrations. Finally the membrane permeating efficiency of 4′-phosphopantetheine was measured using a Parallel Artificial Membrane Permeability Assay (PAMPA assay) (Mensch et al., Eur. J. Pharmaceutics Biopharmaceutics 74:495-502 (2010)). 4′-Phosphopanteheine but not CoA was demonstrated to cross the artificial membrane (FIG. 13E-13F). Altogether, these results point to a capacity of 4′-phosphopanteheine to permeate membranes via passive diffusion.

### Example 6. External Supplementation of CoA Rescues Mutant Phenotypes Associated with dPANK/fbl and dPPCDC but not dCOASY

The prior data show that CoA from external sources can replenish intracellular CoA levels through its hydrolysis product 4′-phosphopantetheine and subsequent conversion back to CoA. The most likely candidate for the latter conversion is the last bifunctional enzyme of the classic CoA biosynthetic pathway: COASY.

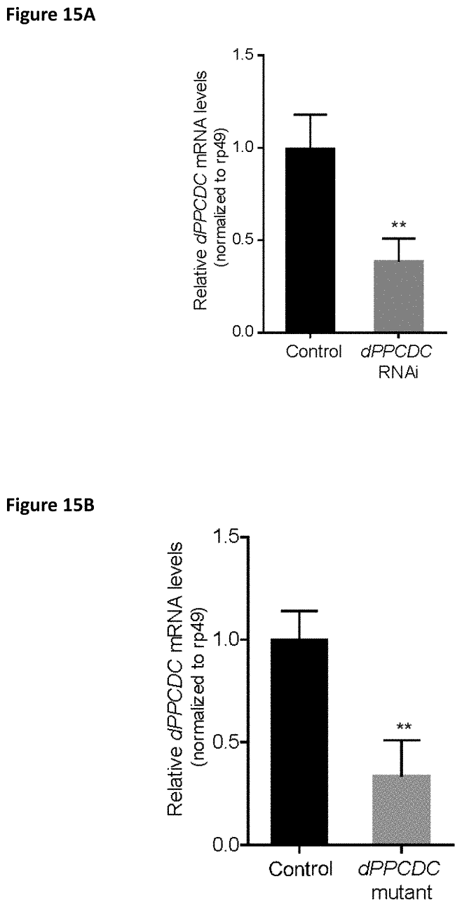

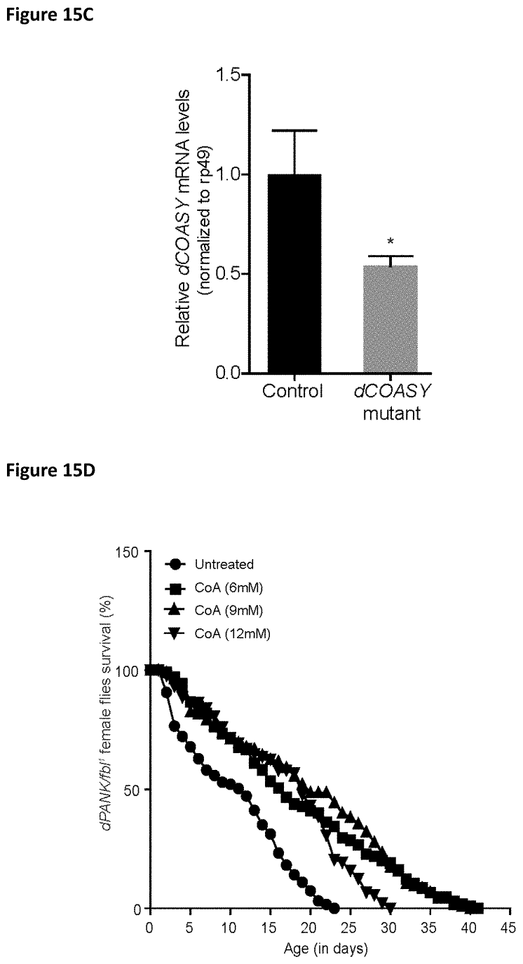

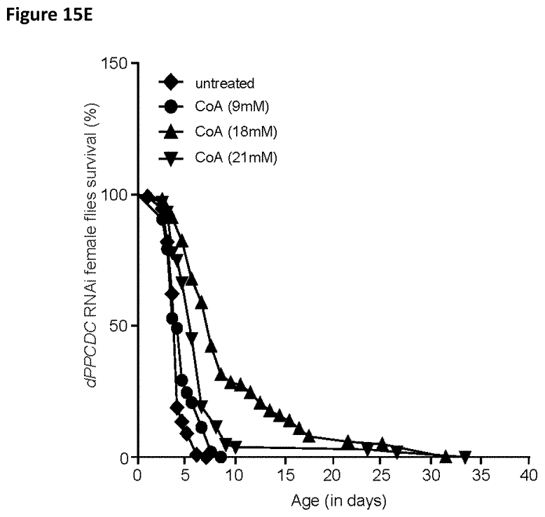

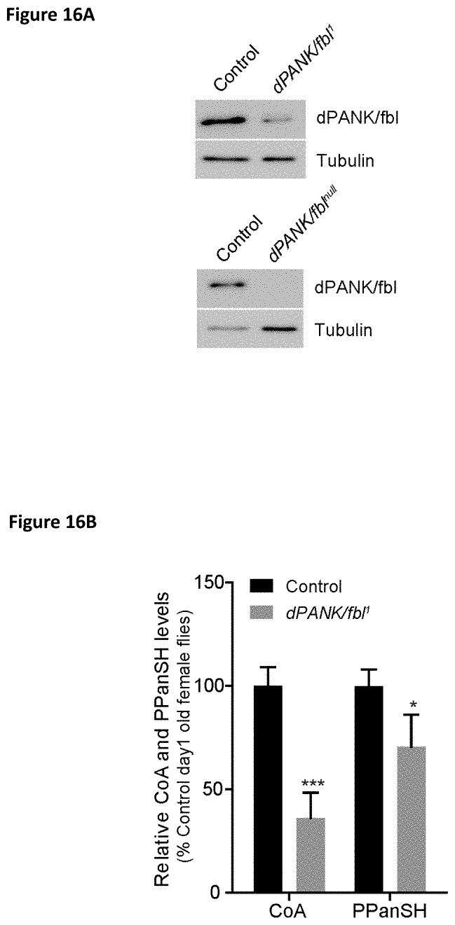

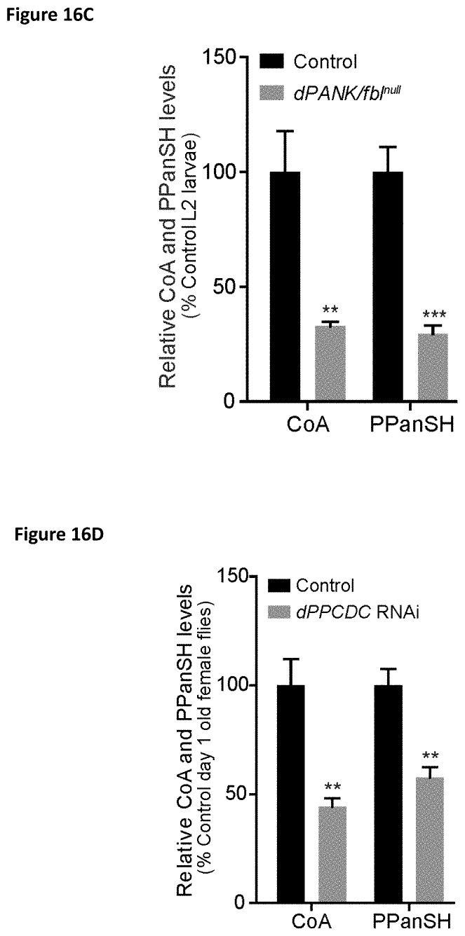

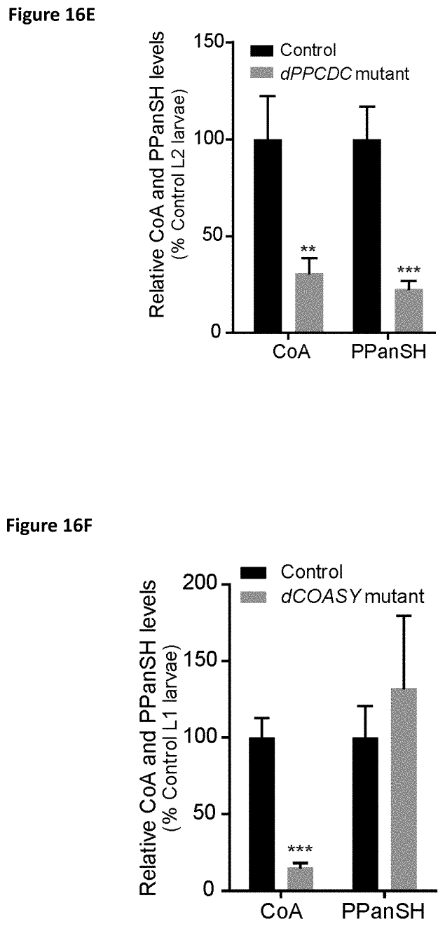

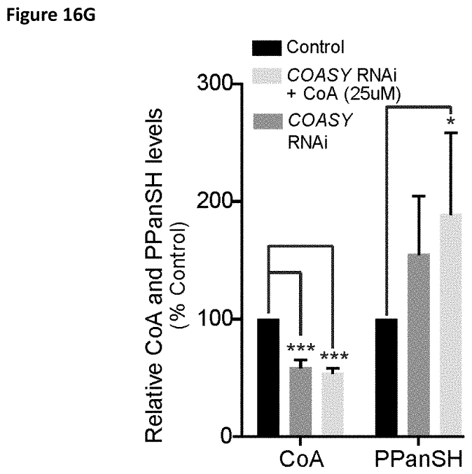

This hypothesis predicts that CoA but not Vitamin B5 can rescue phenotypes caused by mutations in genes encoding enzymes upstream of 4′-phosphopantetheine in the CoA pathway. CoA would not be predicted to rescue COASY mutant phenotypes. In the Drosophila genome, single orthologs have been identified for all the enzymes involved in CoA biosynthesis (Bosveld et al, 2008 supra), including dPANK/fbl, dPPCDC and dCOASY. A set of Drosophila strains was obtained, carrying either deleterious mutations in genes encoding these enzymes or carrying a UAS-RNAi construct. Homozygous mutants or flies ubiquitously expressing the RNAi construct show a downregulation of mRNA levels (FIGS. 15A-15C) or protein levels (FIG. 16A) of these enzymes. CoA and 4′-phosphopantetheine levels were also significantly reduced in all conditions (FIG. 16B-16E), with the exception of dCOASY mutants, which showed a significant reduction of CoA, but not 4′-phosphopantetheine (FIG. 16F).

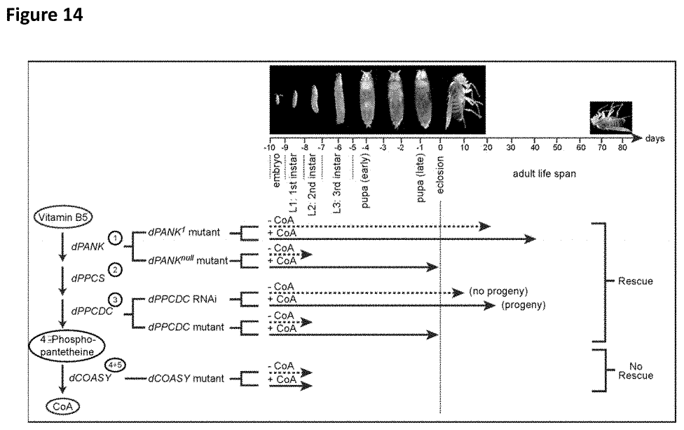

It should be stressed that not all mutants with defects in CoA biosynthesis enzymes show an identical phenotype, which can be explained by the type of Drosophila lines (RNAi expressing lines, hypomorphic or null mutants) used. This has been reported previously not only for Drosophila but also for other organisms (Bosveld et al, 2008 supra; and Rubio, Plant Physiol. 148:546-556 (2008)). Regardless of the severity and developmental stage in which the phenotypes manifest, the determination of the rescue potential of CoA in the available mutants is a valuable tool to test the above hypothesis. A scheme of the hypothesis, Drosophila life span and the phenotypes of the Drosophila lines used are presented in FIG. 14.

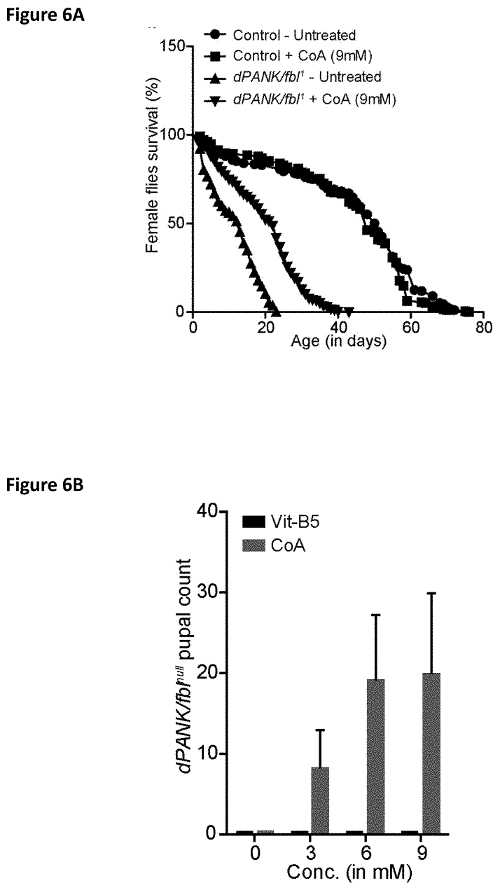

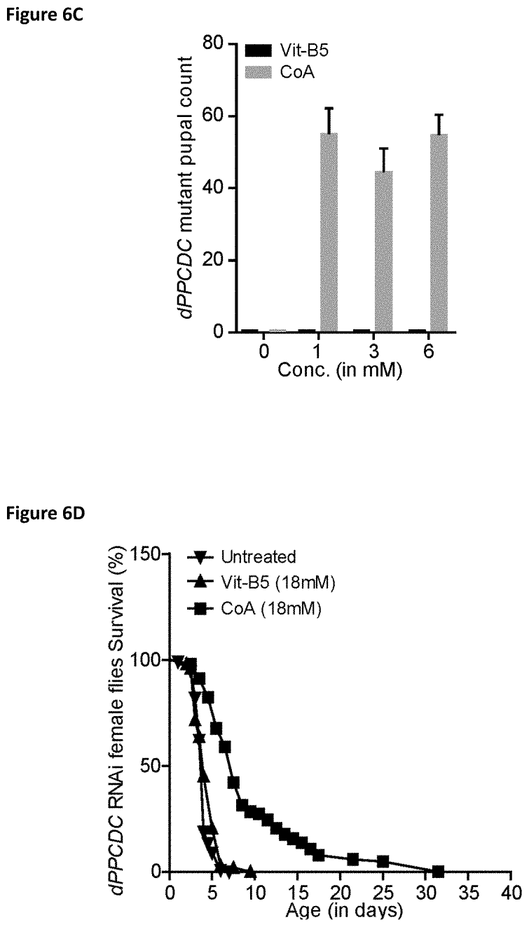

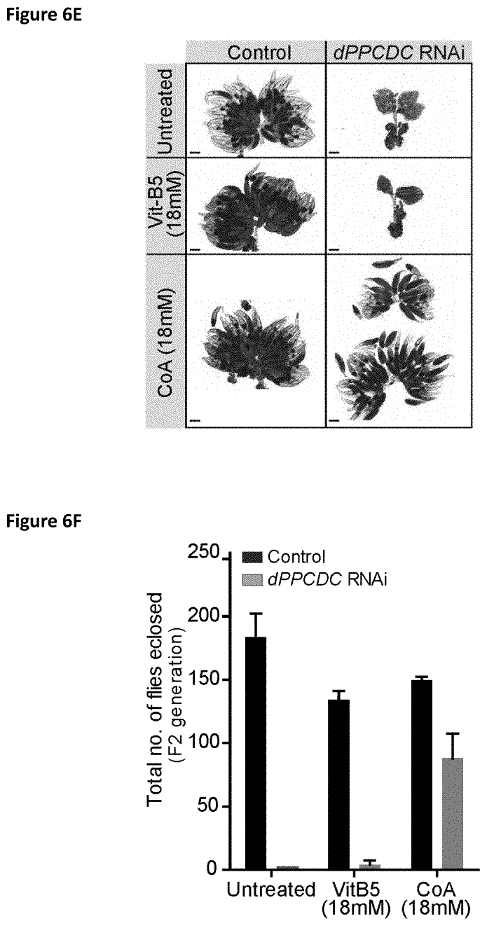

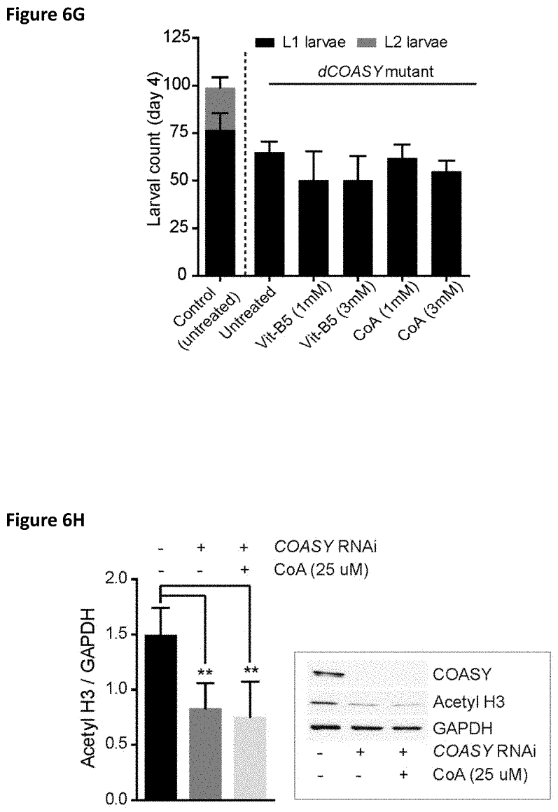

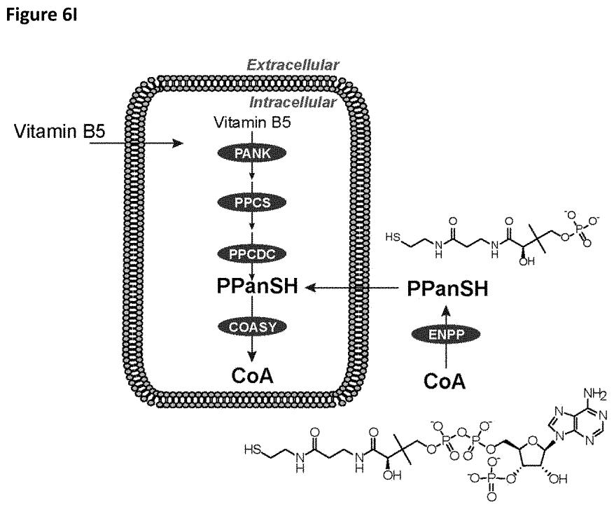

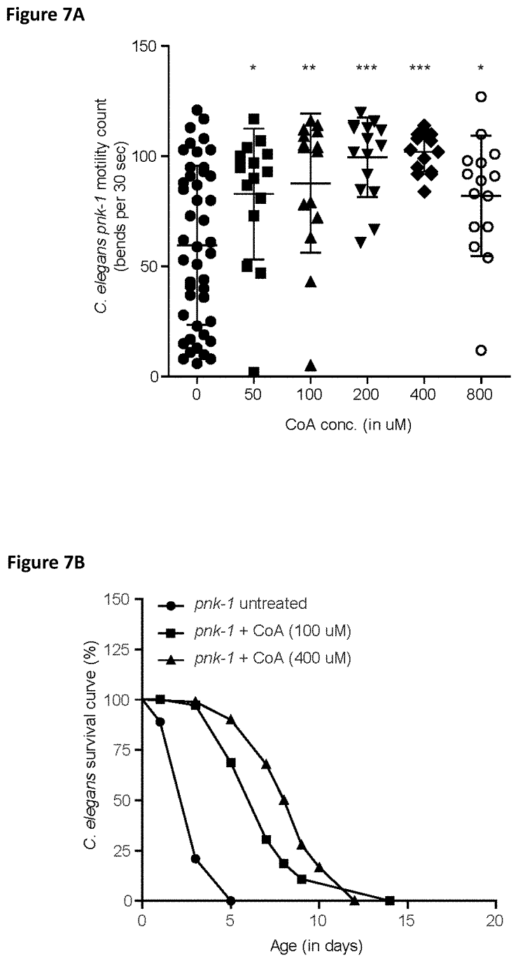

Two Drosophila mutants were available for dPANK/fbl; the hypomorphic dPANK/fbl1 and the null mutant dPANK/fblnull (Rana et al, Proc. Natl. Acad. Sci. USA 107:6988-6993 (2010)). Homozygous dPANK/fbl1 mutants showed reduced levels of dPANK/Fbl protein, and in homozygous dPANK/fblnull mutants, levels of dPANK/Fbl protein were below the level of detection (FIG. 16A). Homozygous dPANK/fbl1 mutants had a shortened adult lifespan (FIG. 6A, 15D), while homozygous dPANK/fblnull mutants only develop until an early L2 larval stage and pupae were not observed (FIG. 6B). Addition of CoA to the food of the homozygous dPANK/fbl1 mutants increased the life span from 20 to 40 days (FIG. 6A, FIG. 15D), and CoA addition to the food of homozygous dPANK/fblnull mutants extended development from the L2 stage to early pupal development (FIG. 6B).

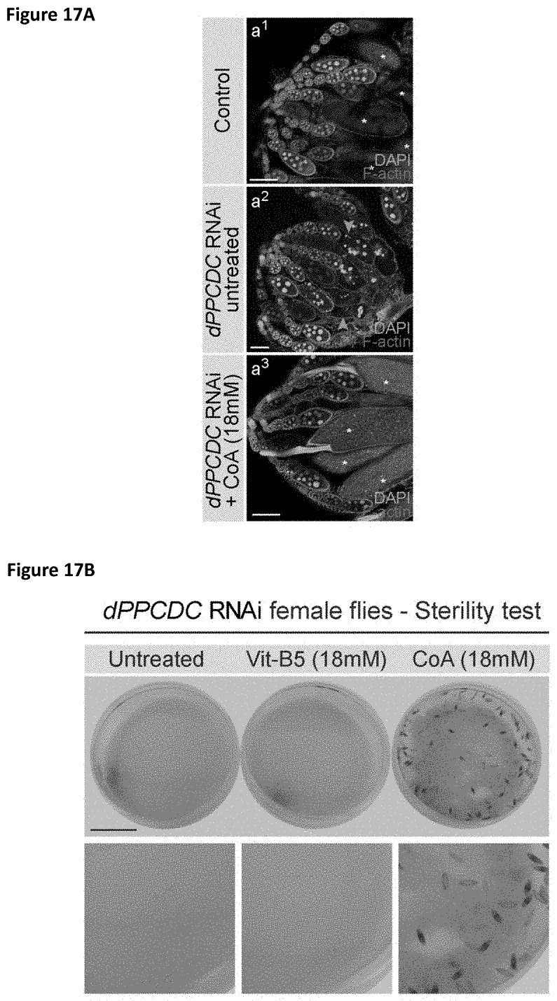

The enzyme dPPCDC catalyzes the third step of the CoA biosynthesis pathway. A UAS-RNAi line (‘dPPCDC RNAi’) as well as a dPPCDC mutant were obtained and rescue by CoA assessed as above. Homozygous dPPCDC mutants showed lethality at early second instar larval stage L2 (FIG. 12C). dPPCDC RNAi expressing flies showed a milder phenotype; adult flies were viable, but had a reduced lifespan (FIG. 6D). Females were sterile, producing no eggs (FIG. 6E, FIG. 17A). Addition of CoA to the food of homozygous dPPCDC mutants extended larval development to late pupal stage (FIG. 6C). Addition of CoA to the food of dPPCDC RNAi expressing flies increased the lifespan from 10 days to 30 days (FIG. 6D, FIG. 15E). Additionally, the females produced viable eggs that resulted in offspring (FIG. 6E, 6F, 17B).

A mutant line of the bifunctional enzyme dCOASY, downstream of 4′-phosphopantetheine was also tested. Homozygous dCOASY mutants develop until first instar larval stage. Addition of CoA to the food did not result in a significant rescue (FIG. 6G).

Vitamin B5 was added to the food as a negative control for all rescue experiments. This did not result in any significant rescue of the phenotypes. A summary of the rescue with CoA in all Drosophila lines is presented in FIG. 14.

Additionally, RNAi was used to downregulate COASY in mammalian HEK293 cells. Under these conditions, the levels of COASY protein (FIG. 6H), CoA (FIG. 16G) and histone acetylation were significantly reduced (FIG. 6H). As in dCOASY mutants, levels of 4′-phosphopantetheine remained unaltered in COASY-compromised mammalian cells (FIG. 16G). Addition of CoA to the medium neither rescued the COASY RNAi-induced decrease in intracellular CoA levels (FIG. 16G) nor restored histone acetylation levels (FIG. 6H). This is in agreement with the above hypothesis that impairment from defects in enzymatic steps downstream of 4′-phosphopantetheine cannot be rescued by exogenous CoA.

Taken together, these results demonstrate that impairment of the CoA biosynthetic pathway by genetic manipulation can give rise to highly complex pleiotropic effects affecting lifespan, development and fecundity. These phenotypes can be (partially) rescued by the addition of CoA to the food of the animals, which is then hydrolyzed to 4′-phosphopantetheine which crosses the plasma membrane via passive diffusion before being converted back to CoA intracellularly, a step requiring COASY (FIG. 6I).

The above experiments can be further confirmed using 4′-phosphopantetheine in place of CoA.

### Example 6. Testing the Physiological Effect of 4′-Phophopantetheine

One of skill in the art would still need to test whether the model described herein (FIG. 6I) occurs physiologically or whether it is artificially provoked by manipulating concentrations of extracellular CoA. The level of CoA and 4′-phosphopantetheine in most extracellular environments and in food is currently unknown. However, compared to CoA concentrations in cytoplasm [0.02-0.14 mM] and mitochondria [2.2-5 mM] (Horie et al, J. Biochem. 99:1345-1352 (1986)), the concentrations used in the experiments described herein (μm range) are relatively low.

Bacteria are able to excrete, but not take up 4′-phosphopantetheine from their environment, suggesting that bacteria-derived 4′-phosphopantetheine may be present in the digestive system (Jackowski et al., J. Bacteriol. 158:115-120 (1984)).

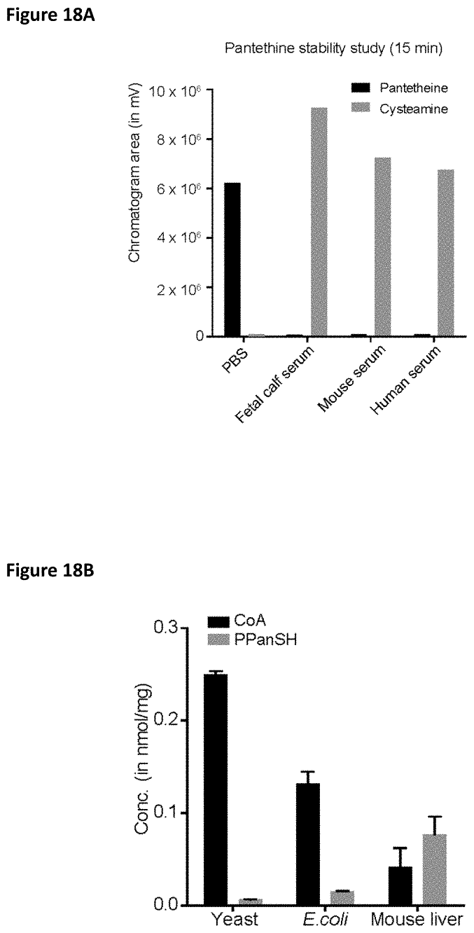

Additionally, full null Drosophila PANK/fbl mutants still display detectable levels of CoA (FIG. 16C). The source of this CoA is unclear, and it may come from maternal sources, bacterial excretion in the Drosophila digestive system, via the food (FIG. 18B) or other external sources.

Furthermore, fresh serum derived from control mice contained endogenous 4′-phosphopantetheine (FIGS. 10A-10C), indicating the presence of an available pool of a CoA precursor that can be transported from one organ to another.

In addition to being a source for intracellular CoA or extracellular CoA, 4′-phosphopantetheine might also have signaling functions in that CoA has an effect on platelet aggregation and vasoconstriction (Coddou et al, FEBS Lett. 536:145-150 (2003); Davaapil et al, Biochem. Soc. Trans. 42:1056-1062 (2014); Lascu et al, Biochem. Biophys. Res. Comm. 156:1020-1025 (1988); Lin et al, Biochim. Biophys. Acta. 428:45-55 (1976); and Manolopoulous et al, Platelets 19:134-145 (2008)). The results disclosed herein suggest that these effects, which have been attributed to CoA, may in fact be from 4′-phosphopantetheine. Future experiments are required to demonstrate the presence and possible impact of a net flow of CoA between organelles, cells and organisms (such as between intestine bacteria to the host).

### Example 7. Rescue Potential of S-Acetyl-4′-Phosphopantetheine in Primary Patient Fibroblast Model of Medium-Chain Acyl-CoA Dehydrogenase (MCAD) Deficiency

Medium-chain acyl-CoA dehydrogenase (MCAD) deficiency is a condition in which the body's capacity to break down fats with medium chain lengths is impaired, caused by mutations in the ACADM gene, which can lead to hypoglycaemia, and liver dysfunction. Left untreated, it can lead to seizures, coma and other serious health problems, with acute symptoms often preceded by extended periods of fasting or an infection with vomiting. Impaired metabolism were observed through functional measurements of respiration using a Seahorse XF Analyzer, where oxygen consumption rate (OCR) reflects oxidative respiration.

A study was performed to test the Rescue potential of S-acetyl-4′-phosphopantetheine in primary patient fibroblast model of MCAD deficiency. MCAD patient fibroblast cell lines (genotyped as containing homozygous K304E mutations in ACADM) were subject to a mitochondrial stress test according to standard protocol with a cell seeding density of 30 k cells/well (n=2). Rescue potential was assessed by increase in reserve capacity: defined as the difference between basal and maximal OCR, controlled by subtracting values for non-mitochondiral respiration (after rotenone treatment). The study was performed in two replicates. Rotenone was used as a positive control to evaluate cell line response, and generated expected profiles of ETC inhibition for all cell lines (data not shown).

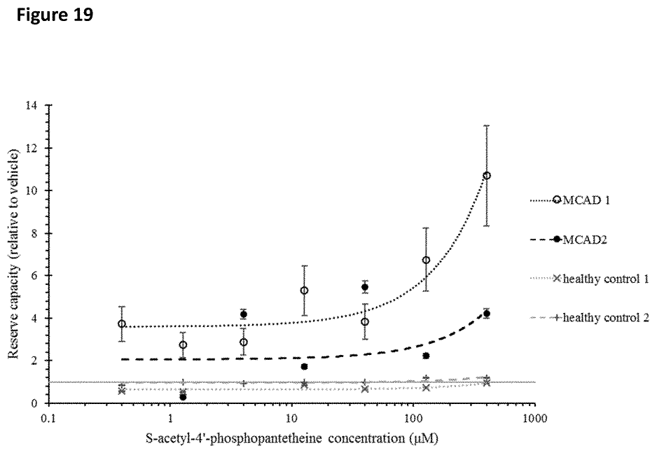

As shown in FIG. 19, upon treatment with S-acetyl-4′-phosphopantetheine, MCAD fibroblasts have an improved spare respiratory capacity (average basal OCR: MCAD 46.95 pmol min⁻¹; healthy controls 113.39 pmol min⁻¹). Data is shown relative to vehicle treated control. Systematically outlying values caused by seeding errors, port failures, or values within background were excluded from analysis. The results demonstrated a reduced basal oxidative respiration, and reduced spare respiratory capacity, compared to fibroblasts from gender matched apparently healthy controls.

The study thereby shows that an active derivative of 4′-phosphopantetheine (e.g., S-acetyl-4′-phosphopantetheine) may increase the ability of the defective human MCAD-cells to cope with energetic demands of maximal respiration stimulated by carbonilcyanide p-triflouromethoxyphenylhydrazone (FCCP).

### Example 8. S-Acetyl-4′-Phosphopantetheine Increases Basal Oxidative Respiration in Primary Fibroblast Cultures

Propionic acidemia (PA) deficiency is a condition in which the body's capacity to break down certain proteins and lipids is impaired, caused by mutations in PCCA or PCCB resulting in insufficient propionyl-CoA carboxylase. MCAD deficiency is a condition in which the body's capacity to break down fats with medium chain lengths is impaired, caused by mutations in the ACADM gene. Due to the role of CoA in both catabolism and energy production, both PA and MCAD are hypothesised to suffer from metabolic deficiencies.

A study was thus performed to test the ability of S-acetyl-(S)-4′-phosphopantetheine to facilitate increased basal oxidative respiration in primary fibroblast cultures from patients diagnosed with MCAD deficiency and PA deficiency. Impaired metabolism were observed through functional measurements of respiration using a Seahorse XF Analyzer, where oxygen consumption rate (OCR) reflects oxidative respiration.

Patient derived cell lines were subject to a mitochondrial stress test according to standard protocol with a cell seeding density of 30 k cells/well (n=2) in glucose free media. After incubation with various concentrations of S-acetyl-4′-phosphopantetheine or vehicle for 24 h, rescue potential was assessed by increase in basal OCR (average of six readings between 30-70 min), relative to control: basal OCR from vehicle treatment was set to 1.0. Rotenone was used as a positive control to evaluate cell line response, and generated expected profiles (data not shown). Experiment was performed in two replicates: systematically outlying values caused by seeding errors, port failures, or values within background were excluded from analysis.

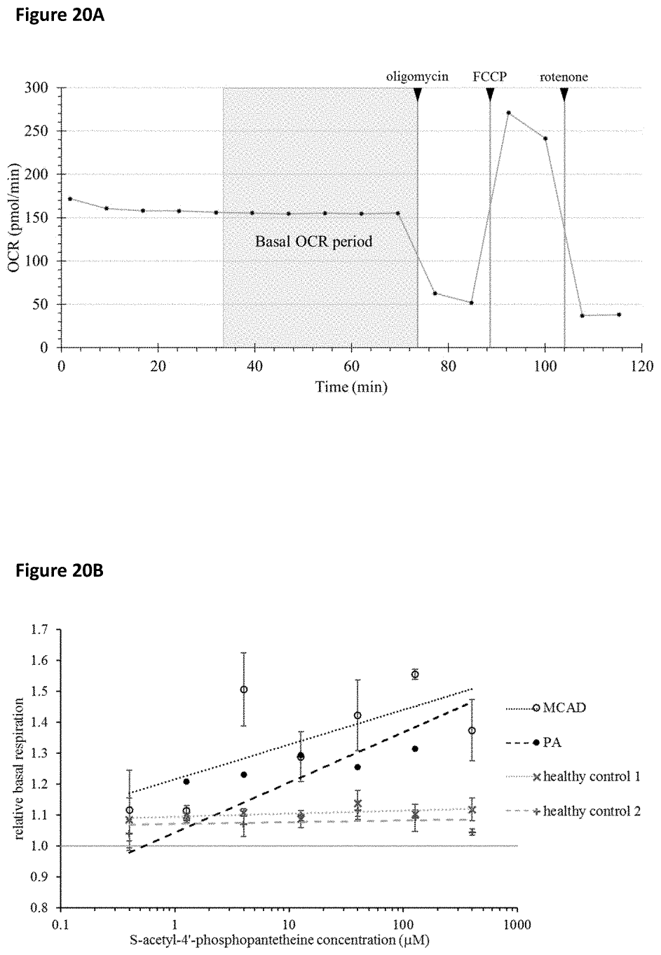

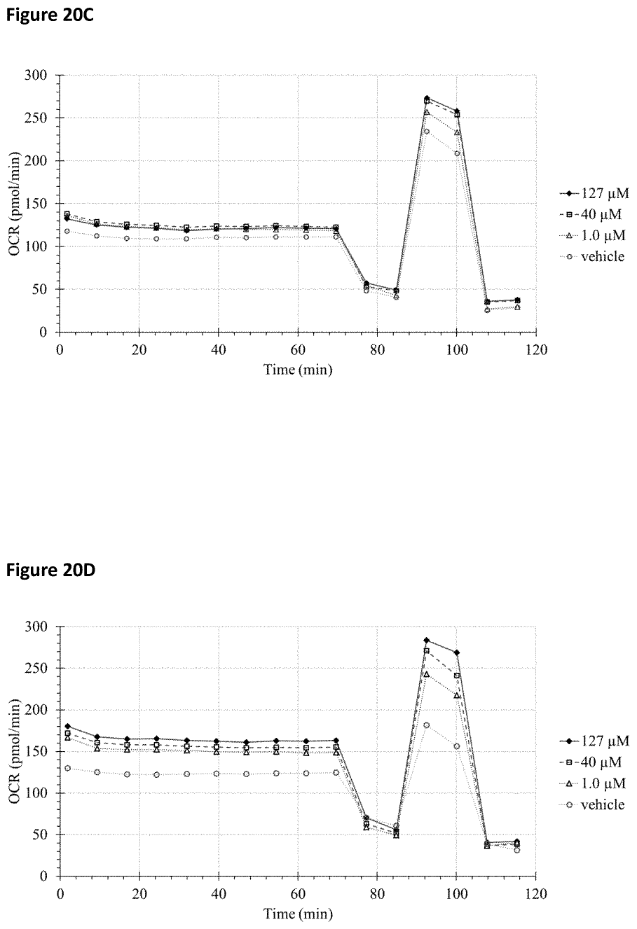

As shown in FIGS. 20A-20D, upon 24 h treatment with S-acetyl-4′-phosphopantetheine, primary fibroblasts exhibit consistently elevated basal OCR levels, relative to vehicle controls. Moreover, this effect was observed more strongly in MCAD and PA patient fibroblasts.

This study thus demonstrates that an active derivative of 4′-phosphopantetheine (e.g., 5-acetyl-4′-phosphopantetheine) may facilitate the increased basal oxidative respiration in primary fibroblast cultures from patients diagnosed with MCAD deficiency and PA deficiency. Such mechanism may benefit subjects with in inborn errors of metabolism, including propionic acidemia (PA) deficiency and medium-chain acyl-CoA dehydrogenase (MCAD) deficiency.

### Example 9. Rescue Potential of (S)-Acetyl-4′-Phosphopantetheine in Drosophila Model of Very-Long-Chain Acyl-CoA Dehydrogenase (VLCAD) Deficiency

VLCAD deficiency is a condition in which the body is unable to break down fats with chain lengths of 12-16 carbons, caused by mutations in the ACADVL gene, which can lead to hypoglycaemia, lethargy and myasthenia, and well as serious complications involving the liver and heart. Problems related to VLCAD deficiency can be triggered by periods of fasting, illness, and exercise.

A study was performed to test the Rescue potential of S-acetyl-4′-phosphopantetheine in drosophila model of very-long-chain acyl-CoA dehydrogenase (VLCAD) deficiency.

The drosophila gene CG7461 is considered to be a good ortholog of ACADVL, as determined by Fly DIOPT (DRSC Integrative Ortholog Prediction Tool). RNAi approaches are an established method of modelling various diseases, and the GD stock library at the Vienna Drosophila Resource Centre (VDRC) contains a knock-down strain for CG7461 (VDRC ID 28028). Down-regulation of CG7461 by RNAi, results in reduced viability when metabolically challenged with starvation.

Five virgin females from Act5C-GAL4 (RNAi expression driver line) and ten virgin males from UAS-GD 28028 (RNAi knock-down of CG7461) were crossed, to generate mutant progeny. In a similar manner, control flies were generated for the RNAi driver line (Gal4 control: Act5C-GAL4×GD 60000) and the upstream activating sequence (UAS control: UAS-GD 28028×Iso 31). From their offspring, 3-day old adult flies were allowed to feed for 24 h on glucose with and without 5 mM (S)-acetyl-4′-phosphopantetheine, then incubated on 2% agar medium without media. Dead flies were counted every 6 hours (up 90 hours) to obtain % survival over time. Rescue potential was assessed by the ability to survive in starvation conditions, expressed as the area under the curve (calculated by trapezium rule) of the cumulative frequency, relative to each control strain.

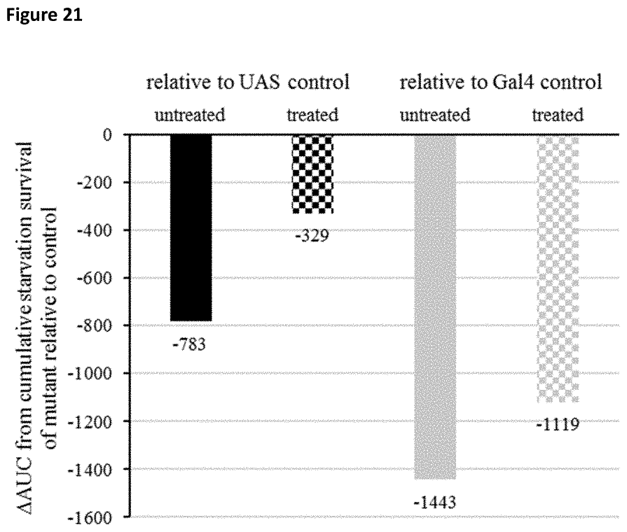

As shown in FIG. 21, upon treatment with 5 mM (S)-acetyl-4′-phosphopantetheine, the mutants impaired ability to survive starvation relative to control flies, was partially recovered. Experiment was performed in 12 replicates, with an average cumulative number of 114 flies in each group. The study thereby demonstrates that, an active derivative of 4′-phosphopantetheine (e.g., S-acetyl-4′-phosphopantetheine) may partially recover the impaired capacity of an in vivo drosophila model of a fatty acid catabolism disorder to cope with starvation, relative to that of the control flies.

### Example 10. Rescue Potential of S-Acetyl-4′-Phosphopantetheine in Drosophila Model of 3-Methylcrotonyl-CoA Carboxylase (3-MCC) Deficiency

3-Methylcrotonyl-CoA carboxylase (3-MCC) deficiency is an inherited disorder affecting leucine catabolism, caused by mutations in the MCCC1 or MCCC2 gene, which can lead to delayed development, seizures, and coma.

A study was performed to test the rescue potential of S-acetyl-4′-phosphopantetheine in drosophila model of 3-MCC deficiency.

The drosophila gene CG34404 is considered to be a good ortholog of both MCCC1 and MCCC2, as determined by Fly DIOPT (DRSC Integrative Ortholog Prediction Tool). RNAi approaches are an established method of modelling various diseases, and the KK stock library at the Vienna Drosophila Resource Centre (VDRC) contains a knock-down strain for CG34404 (VDRC ID 103335). Down-regulation of CG34404 by RNAi, causes developmental delay.

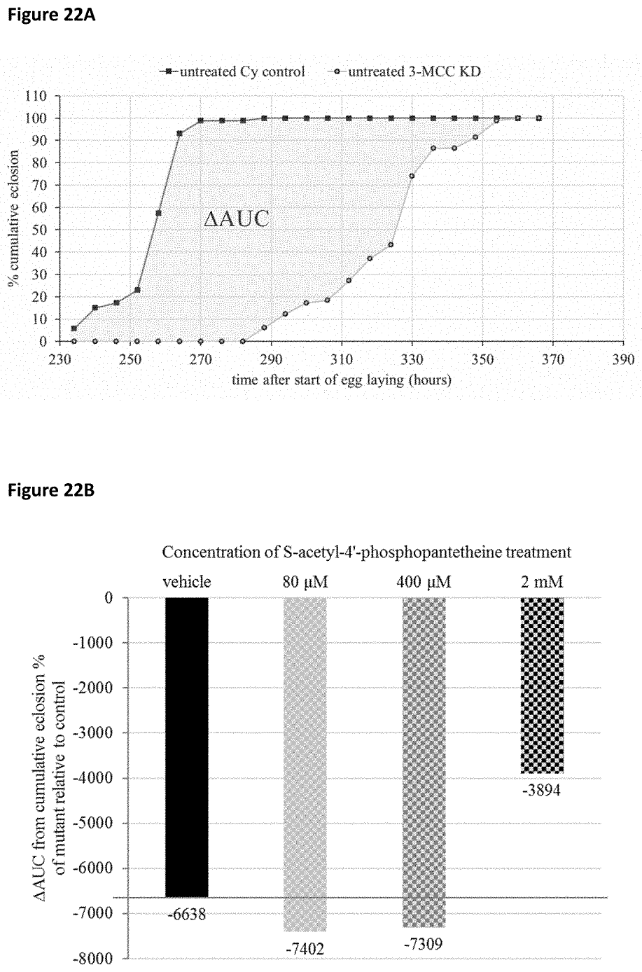

Five virgin females from UAS-KK 103335 (RNAi knock-down of CG34404) and ten virgin males from Act5C-GAL4 (heterozygous RNAi expression driver line with CyO balancer) were crossed, and allowed to lay for a period of 24 h in vials containing drosophila media with and without S-acetyl-4′-phosphopantetheine. Rescue potential was assessed by the number of eclosed male mutant or control flies every 6 h, as a percentage of total eclosed flies of each genotype, expressed as the AAUC (calculated by trapezium rule) of the cumulative eclosion relative to the control strain, as shown in FIG. 22A. Further, the region of relative AUCs are plotted in FIG. 22B under treatment conditions.

As seen in FIGS. 22A-22B, treatment with 2 mM S-acetyl-4′-phosphopantetheine was able to partially rescue viability in CG34404 down-regulated drosophila, by reducing the observed developmental delay by the equivalent of 27 cumulative fly days. The observation that treatment with S-acetyl-4′-phosphopantetheine resulted in some toxicity independent of RNAi expression is in line with previous findings that increasing concentrations of CoA metabolites are not as well tolerated in drosophila as in mammalian species. This suggests a sufficient rescue potential at 2 mM to compensate for both the genetic developmental delay, and mild background toxicity. Experiment was performed in eight replicates, with an average cumulative number of 76 flies of each genotype, for each treatment condition.

This study thereby demonstrates that an active derivative of 4′-phosphopantetheine (e.g., S-acetyl-4′-phosphopantetheine) may partially restore viability in an in vivo drosophila model of an amino acid catabolism disorder.

### EQUIVALENTS

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

1-44. (canceled)

45. A method of treating a diseased subject having a Coenzyme A sequestration, toxicity or redistribution (CASTOR) disease, comprising administering to the diseased subject an effective amount of an active derivative of 4′-phosphopantetheine, wherein the active derivative of 4′-phosphopantetheine is a compound of Formula (I): 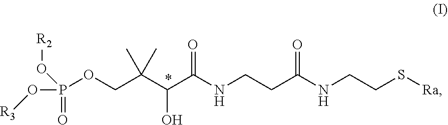
a pharmaceutically acceptable salt thereof, or a solvate thereof, wherein:
Ra is H, 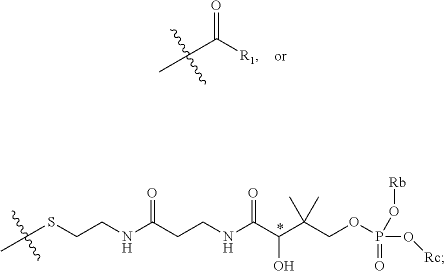
R₁ is H, substituted or unsubstituted alkyl, substituted or unsubstituted alkenyl, substituted or unsubstituted cycloalkyl, substituted or unsubstituted aryl, substituted or unsubstituted arylalkyl, substituted or unsubstituted non-aromatic heterocyclyl, substituted or unsubstituted heterocyclyl, substituted or unsubstituted heterocyclylalkyl, COR₁₁, C(O)OR₁₁, C(O)NR₁₁R₁₂, C═NR₁₁, CN, OR₁₁, OC(O)R₁₁, NR₁₁R₁₂, NR₁₁C(O)R₁₂, NO₂, N═CR₁₁R₁₂, or halogen;
R₂, R₃, Rb, and Rc is each independently selected from the group consisting of H, methyl, ethyl, phenyl, acetoxymethyl (AM), pivaloyloxymethyl (POM), 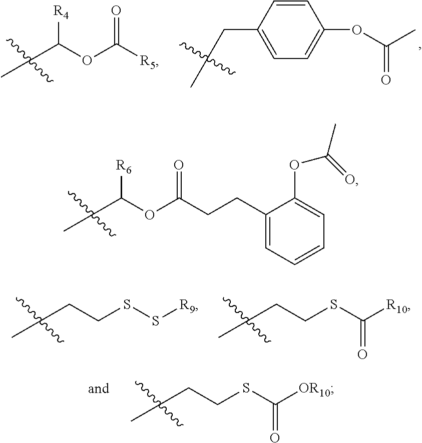
or
R₂ and R₃, or Rb and Rc, jointly form a structure selected from the group consisting of 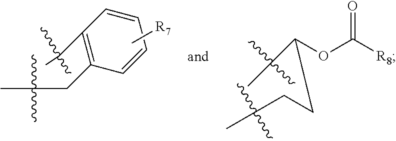
wherein
R₄ is H or alkyl;
R₅ is H or alkyl;
R₆ is H, alkyl, or CH₂(CO)OCH₃;
R₇ is H, alkyl, or halogen;
R₈ is H or alkyl;
R₉ is H or alkyl;
R₁₀ is H or-alkyl; and
R₁₁ and R₁₂ each is hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, or halogen; and
wherein the diseased subject does not have a pantothenate kinase-associated neurodegeneration (PKAN) disease.

46-52. (canceled)

53. The method of claim 45, wherein the CASTOR disease is associated with inhibition of one or more pantothenate kinases by one or more acyl Coenzyme A (acyl-CoA) species.

54. The method of claim 45, wherein the CASTOR disease is associated with accumulation of one or more acyl Coenzyme A (acyl-CoA) species in the diseased subject to amounts greater than that of a healthy subject not having the CASTOR disease.

55. (canceled)

56. The method of claim 45, wherein the CASTOR disease is associated with impaired or inhibited degradation of the one or more acyl-CoA species in the diseased subject.

57. The method of claim 45, wherein the one or more acyl-CoA species are not acetyl Coenzyme A (acetyl-CoA).

58. The method of claim 45, wherein the CASTOR disease is associated with accumulation of one or more fatty acids in the diseased subject to amounts greater than that of a healthy subject not having the CASTOR disease.

59. The method of claim 45, wherein the CASTOR disease is associated with impaired or inhibited degradation of the one or more fatty acids in the diseased subject.

60. The method of claim 45, wherein the CASTOR disease is selected from the group consisting of medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

61-62. (canceled)

63. The method of claim 60, wherein the CASTOR disease is selected from the group consisting of medium chain acyl-CoA dehydrogenase deficiency, short chain acyl-CoA dehydrogenase deficiency, very long chain acyl-CoA dehydrogenase deficiency, and D-bifunctional protein deficiency.

64-67. (canceled)

68. The method of claim 60, wherein the CASTOR disease is selected from the group consisting of Glutaric acidemia type 1, methylmalonic academia, propionyl-CoA carboxylase deficiency, propionic academia, 3-methylcrotonyl carboxylase deficiency, and isovaleryl-CoA dehydrogenase deficiency.

69-75. (canceled)

76. The method of claim 45, wherein the compound of Formula (I) is a compound of Formula (Ia):

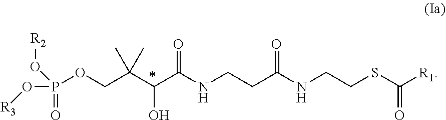

77. The method of claim 45, wherein R₁ is C₁-C₁₀ alkyl.

78. (canceled)

79. The method of claim 77, wherein R₁ is methyl.

80. The method of claim 45, wherein at least one of R₂ and R₃ is H.

81-82. (canceled)

83. The method of claim 45, wherein the active derivative of 4′-phosphopantetheine is 4′-phosphopantetheine or a pharmaceutically acceptable salt thereof.

84. The method of claim 45, wherein the active derivative of 4′-phosphopantetheine is S-acyl-4′-phosphopantetheine or a pharmaceutically acceptable salt thereof.

85. (canceled)

86. The method of claim 45, wherein the active derivative of 4′-phosphopantetheine is S-acetyl-4′-phosphopantetheine.

87. (canceled)

88. The method of claim 45, wherein the active derivative of 4′-phosphopantetheine is a calcium salt of S-acetyl-4′-phosphopantetheine.

89-176. (canceled)

177. A pharmaceutical kit for use in the treatment of a diseased subject having a Coenzyme A sequestration, toxicity or redistribution (CASTOR) disease, comprising an effective amount of the active derivative of 4′-phosphopantetheine.

178. A method of synthesizing the active derivative of 4′-phosphopantetheine, comprising the steps of:
i) chemically treating pantothenic acid with S-tritylcysteamine to form S-tritylpantetheine;
ii) chemically treating S-tritylpantetheine with dibenzylchlorophosphate to form S-trityl-4′-dibenzylphosphopantetheine; and
iii) chemically treating S-trityl-4′-dibenzylphosphopantetheine to form 4′-phosphopantetheine.

179-181. (canceled)


SEQUENCE LISTING (5 sequences)

SEQ ID NO: 1 - DNA, 40 bp, Artificial Sequence
  Primer
tgcacctgcgatgaataccctcggctgaaaggcggataac

SEQ ID NO: 2 - DNA, 20 bp, Artificial Sequence
  Primer Sequence
ggctgtgcggcggattattg

SEQ ID NO: 3 - DNA, 20 bp, Artificial Sequence
  Primer Sequence
cgggttaaaggctgctctgg

SEQ ID NO: 4 - DNA, 18 bp, Artificial Sequence
  Primer Sequence
gcaccaagcacttcatcc

SEQ ID NO: 5 - DNA, 18 bp, Artificial Sequence
  Primer Sequence
cgatctcgccgcagtaaa